(19) United States
(12) Patent Application Publication
(10) Pub. No.: US 2025/0057828 A1
(43) Pub. Date: Feb. 20, 2025

(54) COMBINATION OF AN A2-ADRENOCEPTOR SUBTYPE C (ALPHA-2C) ANTAGONIST WITH A NOREPINEPHRINE REUPTAKE INHIBITOR FOR THE TREATMENT OF SLEEP APNEA

(71) Applicant: BAYER AKTIENGESELLSCHAFT, LEVERKUSEN (DE)

(72) Inventors: Martina DELBECK, Heiligenhaus (DE); Michael HAHN, Langenfeld (DE)

(21) Appl. No.: 18/722,233

(22) PCT Filed: Dec. 20, 2022
(86) PCT No.: PCT/EP2022/086928

§ 371 Date: Jun. 20, 2024

(30) Foreign Application Priority Data
Dec. 22, 2021 (EP) 21216889.2

(51) Int. Cl. A61K 31/4545; A61K 31/222; A61K 45/06

(52) U.S. Cl. CPC A61K 31/4545; A61K 31/222; A61K 45/06

(57) ABSTRACT

The present invention relates to a combination of a muscarinic receptor antagonist and α2-Adrenoceptor subtype C (alpha-2C) antagonists, in particular substituted heterocyclic carboxamides of formula (I) for the treatment and/or prophylaxis of sleep-related breathing disorders, preferably obstructive and central sleep apneas and snoring.

The present invention relates to a combination of α2-Adrenoceptor subtype C (alpha-2C) antagonists, in particular substituted heterocyclic carboxamides of formula (I) and a muscarinic receptor antagonist for the treatment and/or prophylaxis of sleep-related breathing disorders, preferably obstructive and central sleep apneas and snoring.

### BACKGROUND OF THE INVENTION

Obstructive sleep apnoea (OSA) is a sleep-related respiratory disorder which is characterized by repetitive episodes of obstruction of the upper airways. When breathing in, the patency of the upper airways is ensured by the interaction of two opposite forces. The dilative effects of the musculature of the upper airways counteract the negative intraluminal pressure, which constricts the lumen. The active contraction of the diaphragm and the other auxiliary respiratory muscles generates a negative pressure in the airways, thus constituting the driving force for breathing. The stability of the upper respiratory tract is substantially determined by the coordination and contraction property of the dilating muscles of the upper airways.

Upper airway collapse in OSA is thought to occur at sleep onset because of the reduction of activity of several upper airway dilator muscles, which as a consequence are unable to maintain the anatomically vulnerable airway open. However, some upper airway dilator muscles, including the genioglossus muscle, which is the most important of the dilating muscles of the upper respiratory airway and which is innervated by the hypoglossal nerve, can increase activity during sleep in response to respiratory stimuli, potentially counteracting some of these changes at sleep onset. It was observed that OSA patients have apnea free intervals in which the genioglossus muscle activity is only 25-40% higher compared with sleep phases with frequent obstructive apneas (Jordan A S, White D P, Lo Y L et al., Airway dilator muscle activity and lung volume during stable breathing in obstructive sleep apnea. Sleep 2009, 32(3): 361-8). Noradrenaline is one of the most potent neuromodulators of hypoglossal motoneuron activity (Horner R. L. Neuromodulation of hypoglossal motoneurons during sleep. Respir Physiol Neurobiol 2008, 164 (1-2): 179-196). It is thought, that decreased noradrenergic drive leads to sleep-dependent decline of hypoglossal motoneuron excitability resulting in reduced upper airway dilator muscle activity, especially reduced genioglossus muscle activity.

Alpha2C adrenoceptors regulate the release of noradrenaline from central noradrenergic neurons, they are autoreceptors involved in presynaptic feedback inhibition of noradrenaline (Hein L. et al, Two functionally distinct alpha2-adrenergic receptors regulate sympathetic neurotransmission Nature 1999, 402(6758): 181-184). An increase in the activity of the motoneurons of the hypoglossal nerve through Alpha2c adrenoceptor antagonism can stabilize the upper airways and protect them from collapse and occlusion. Moreover, also snoring can be inhibited through the mechanism of stabilization of the upper respiratory airways [Horner R. L. Neuromodulation of hypoglossal motoneurons during sleep. Respir Physiol Neurobiol 2008, 164 (1-2): 179-196].

For simple snoring, there is no obstruction of the upper airways. By the narrowing of the upper airways, the flow velocity of the inhaled and exhaled air increases. This together with the relaxed muscles causes fluttering of the soft tissues of the mouth and throat in the airflow. This slight vibration generated the typical snoring sounds.

Obstructive snoring (upper airway resistance syndrome, heavy snoring, hypopnea syndrome) is caused by a recurrent partial obstruction of the upper airway during sleep. This results in an increase in airway resistance and thus to an increase in work of breathing with significant intrathoracic pressure fluctuations. The negative intrathoracic pressure development during inspiration can thereby reach values as they occur as a result of a complete airway obstruction in OSA. The pathophysiological effects on the heart, circulation and sleep quality are the same as in obstructive sleep apnea. The pathogenesis is likely the same as in OSA. Obstructive snoring often provides the precursor for OSA (Hollandt J. H. et al., Upper airway resistance syndrome (UARS)-obstructive snoring. HNO 2000, 48(8): 628-634).

Central sleep apnea (CSA) occurs as a result of disturbed brain function or impaired respiratory regulation. CSA is characterized by a lack of drive to breathe during sleep, resulting in repetitive periods of insufficient or absent ventilation and compromised gas exchange. There are several manifestations of CSA. These include high altitude-induced periodic breathing, idiopathic CSA (ICSA), narcotic-induced central apnea, obesity hypoventilation syndrome (OHS), and Cheyne-Stokes breathing (CSB). While the precise precipitating mechanisms involved in the various types of CSA may vary considerably, unstable ventilatory drive during sleep is a principal underlying feature (Eckert D. J. et al., Central sleep apnea: Pathophysiology and treatment. Chest 2007, 131(2): 595-607).

US 2018/0235934 A1 describes methods for treating disorders such as obstructive sleep apnea using agents for promoting hypoglossal motoneuron excitability. As agents for promoting hypoglossal motoneuron excitability a disinhibitor and/or stimulant of central noradrenic neurons is described. In some embodiments the disinhibitor of central noradrenergic neurons is an alpha2-adrenoceptor antagonist such as yohimbine or an alpha2-adrenoceptor subtype A (alpha-2A) antagonists or alpha2-adrenoceptor subtype C (alpha-2C) antagonist. The alpha2-adrenoceptor antagonist are selected from the group consisting of Atipamezole, MK-912, RS-79948, RX 821002, [3H]2-methoxy-idazoxan and JP-1302.

Alpha2C adrenoceptors belong to the family of G-protein coupled receptors. Beside the different Alpha1-adrenoceptors three different Alpha2-adrenoceptor subtypes exist (Alpha2A, Alpha2B and Alpha2C). They are involved in the mediation of several diverse physiologic effects in different tissues upon stimulation by endogeneous catecholamines (epinephrine, norepinephrine), either derived from synapses or via the blood. Alpha2 adrenoceptors play an important physiological role, mainly in the cardiovascular system and in the central nervous system. Alpha2A- and Alpha2C-adrenoceptors are the main autoreceptors involved in presynaptic feedback inhibition of noradrenaline in the central nervous system. The potency and affinity of noradrenaline at the Alpha2C-adrenoceptor is higher than that for the Alpha2A-adrenoceptor. The Alpha2C-adrenoceptor inhibits noradrenaline release at low endogenous concentrations of noradrenaline, while Alpha2A-adrenoceptors inhibit noradrenaline release at high endogenous noradrenaline concentrations (Uys M. M. et al. Therapeutic Potential of Selectively Targeting the α2C-Adrenoceptor in Cognition, Depression, and Schizophrenia—New Developments and Future Perspective. Frontiers in Psychiatry 2017, Aug. 14; 8:144. doi: 10.3389/fpsyt.2017.00144. eCollection 2017).

A further mechanism to increase pharyngeal dilator muscle activity, especially genioglossus muscle activity, is blocking cholinergic transmission in the hypoglossal motor nucleus via muscarinic receptor antagonists. Cholinergic signaling impairs upper airway dilator muscle activity by suppressing glutaminergic input from parahypoglossal premotoneurons to hypoglossal motoneurons and by directly inhibiting hypoglossal motoneurons via muscarinic receptors. (Zhu L. et al., Muscarinic Inhibition of Hypoglossal Motoneurons: Possible Implications for Upper Airway Muscle Hypotonia during REM Slee. J Neurosci 2019, Oct. 2;39(40):7910-7919; X. Liu et al., Opposing muscarinic and nicotinic modulation of hypoglossal motor output to genioglossus muscle in rats in vivo. J Physiol 2005, Jun. 15;565(Pt 3):965-80). A muscarinic receptor antagonist is a type of anticholinergic agent that blocks the activity of the muscarinic acetylcholine receptor (T. Oki et al., Comparative Evaluation of Central Muscarinic Receptor Binding Activity by Oxybutynin, Tolterodine and Darifenacin Used to Treat Overactive Bladder. J Urol 2007, February; 177(2):766-70).

Muscarinic receptor antagonists are described in the literature as agents for the treatment of obstructive airway diseases and urinary bladder dysfunction (Dale P. R. The pharmacological rationale for combining muscarinic receptor antagonists and b-adrenoceptor agonists in the treatment of airway and bladder disease. Curr Opin Pharmacol 2014, 16(100): 31-42).

In WO 2018/200775 and WO 2019/152475 compositions for the treatment of conditions associated with pharyngeal airway muscle collapse while the subject is in a non-fully conscious state, e.g., sleep apnea and snoring, comprising administration of a norepinephrine reuptake inhibitor (NRI) and a muscarinic receptor antagonist are described.

Aryl piperazines as α2-Adrenoceptor subtype C (alpha-2C) antagonists as well as their preparation and the use thereof as a medicament are known from WO 03/082866 A1 where the compounds are disclosed as useful for the treatment for disorders such as disorder propagated by stress, Parkinson's disease, depression, schizophrenia, attention deficit hyperactivity disorder, post-traumatic stress disorder, obsessive compulsive disorder, Tourette's syndrome, blepharospasm or other focal dystonias, temporal lobe epilepsy with psychosis, a drug-induced psychosis, Huntington's disease, a disorder caused by fluctuation of the levels of sex hormones, panic disorder, Alzheimer's disease or mild cognitive impairment. There is nothing disclosed about the use of these compounds in the treatment of sleep-related breathing disorders, preferably obstructive and central sleep apneas and snoring.

Combinations of α2-Adrenoceptor subtype C (alpha-2C) antagonists with TASK-1/TASK-3 antagonists for the treatment of sleep apnea were described in WO2020/225185 and WO2020/225188, respectively.

WO 2021089683 describes substituted heterocyclic carboxamides as inhibitors of adrenoreceptor ADRA2C and their use for the treatment and/or prevention of diseases, in particular for the treatment and/or prevention of breathing difficulties including sleep-induced breathing difficulties such as central and obstructive sleep apnoea, snoring.

The current gold standard treatment for patients with OSA is continuous positive airway pressure (CPAP). The positive airflow pressure that is generated by an airflow turbine pump splints open the upper airway, reversing all potential causes of pharyngeal collapse, thereby preventing hypopneas, apneas and sleep fragmentation. Unfortunately, up to 50% of all patients with OSA do not tolerate CPAP in the long-term (M. Kohler, D. Smith, V. Tippett et al., Thorax 2010 65(9):829-32: Predictors of long-term compliance with continuous positive airway pressure). Therefore, there is still the need to find effective therapeutic agents for the treatment and/or prophalxis of sleep-related breathing disorders such as obstructive sleep apnea. Therefore the object of the present invention is to provide an effective therapeutic agent for the treatment and/or prophalxis of sleep-related breathing disorders, for example of obstructive sleep apnea, central sleep apnea and snoring.

Surprisingly, it has now been found that the combination of an α2-Adrenoceptor subtype C (alpha-2C) antagonist with a muscarinic receptor antagonist inhibits upper airway collapsibility with synergistic efficacy and is thus suitable for the production of medicaments for the use in the treatment and/or prophylaxis of sleep-related breathing disorders, preferably obstructive and central sleep apneas and snoring. It was found that a synergism of the combination of an α2-Adrenoceptor subtype C (alpha-2C) antagonist with a muscarinic receptor antagonist allows lower doses of each treatment.

The present invention relates to combinations of compounds of formula (I)

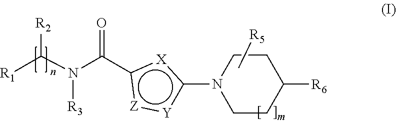

in which
X represents S, N or O;
Y represents N, S or O,
   where, if X represents S, then Y represents N;
   where, if X represents 0, then Y represents N;
Z represents CR₄, O or NR₄,
   where, if X represents N and Y represents N, then Z represents 0;
   where, if X represents S, then Z represents CR₄ or NR₄
R₁ represents 5- or 6-membered heteroaryl, phenyl,
   where 5- to 6-membered heteroaryl may be substituted by 1 to 2 substituents independently of one another selected from the group of (C₁-C₄)-alkyl, (C₁-C₄)-alkoxy, halogen;
      where (C₁-C₄)-alkyl may be up to trisubstituted by halogen,
      where (C₁-C₄)-alkoxy may be up to trisubstituted by halogen,
   where phenyl may be substituted by 1 to 2 substituents independently of one another selected from the group of (C₁-C₄)-alkyl, (C₃-C₅)-cycloalkyl, (C₁-C₄)-alkoxy, cyano, hydroxy, halogen;
      where (C₁-C₄)-alkyl may be up to trisubstituted by halogen,
R₂ represents hydrogen, (C₁-C₄)-alkyl;
   where (C₁-C₄)-alkyl may be up to trisubstituted by halogen, or together with the carbon atom to which R₂ is attached forms a (C₃-C₄)-cycloalkyl ring,
R₃ represents hydrogen, (C₁-C₄)-alkyl,
   where (C₁-C₄)-alkyl may be up to trisubstituted by halogen,
R₄ in CR₄ represents hydrogen, (C₁-C₄)-alkyl, (C₃-C₄)-cycloalkyl, phenyl, halogen;
   where (C₁-C₄)-alkyl may be up to trisubstituted by halogen and phenyl may be substituted by halogen, in NR₄ represents hydrogen, (C₁-C₄)-alkyl, (C₃-C₄)-cycloalkyl, phenyl;
   where (C₁-C₄)-alkyl may be up to trisubstituted by halogen and phenyl may be substituted by halogen,
R₅ represents hydrogen, (C₁-C₄)-alkyl, (C₁-C₄)-alkoxy, halogen,
R₆ represents a group of formula a), b), c), d), e), f) or g)

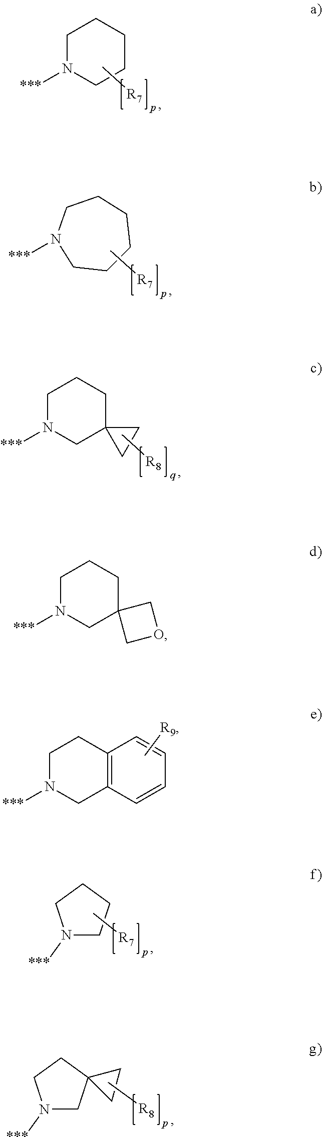

where *** marks the attachment to the adjacent piperidine ring,
where R₇ represents hydrogen, (C₁-C₄)-alkyl, (C₃-C₄)-cycloalkyl, (C₁-C₄)-alkoxy, (C₃-C₄)-cycloalkoxy, phenyl,
   where (C₁-C₄)-alkyl may be substituted by (C₃-C₄)-cycloalkyl, (C₁-C₄)-alkoxy, (C₃-C₄)-cycloalkoxy and up to trisubstituted by halogen,
      where (C₁-C₄)-alkoxy may be substituted by (C₃-C₄)-cycloalkyl and up to trisubstituted by halogen,
      where (C₃-C₄)-cycloalkyl may be substituted by monofluoromethyl, difluoromethyl or trifluoromethyl and up to disubstituted by halogen,
   where (C₁-C₄)-alkoxy may be substituted by (C₃-C₄)-cycloalkyl and up to trisubstituted by halogen,
      where (C₃-C₄)-cycloalkyl may be mono- or disubstituted by halogen,
   where (C₃-C₄)-cycloalkoxy may be up to disubstituted by halogen,
where R₈ represents hydrogen or fluorine,
where R₉ represents hydrogen, (C₁-C₄)-alkyl, (C₁-C₄)-alkoxy, halogen;
   where (C₁-C₄)-alkyl may be substituted by (C₁-C₄)-alkoxy,
n represents 0 or 1,
m represents 0, 1 or 2,
p represents 0, 1 or 2 and
q represents 0, 1 or 2, and
muscarinic receptor antagonists,
and the salts, solvates and solvates of the salts thereof.

Compounds of the invention are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by formula (I) and are cited below as working examples and the salts, solvates and solvates of the salts thereof if the compounds that are encompassed by formula (I) and are mentioned below are not already salts, solvates and solvates of the salts.

Compounds of the invention are likewise N-oxides and S-oxides of the compounds of the formula (I) and the salts, solvates and solvates of the salts thereof.

Preferred salts in the context of the present invention are physiologically acceptable salts of the compounds according to the invention. Also encompassed are salts which are not themselves suitable for pharmaceutical applications but can be used, for example, for the isolation, purification or storage of the compounds of the invention.

A suitable pharmaceutically acceptable salt of the compounds of the present invention may be, for example, an acid-addition salt of a compound of the present invention bearing a sufficiently basic nitrogen atom in a chain or in a ring, such as an acid-addition salt with an inorganic acid, or “mineral acid”, such as hydrochloric acid, hydrobromic acid, hydroiodic acid, sulfuric acid, sulfamic acid, bisulfuric acid, phosphoric acid or nitric acid, for example, or with an organic acid such as formic acid, acetic acid, acetoacetic acid, pyruvic acid, trifluoroacetic acid, propionic acid, butyric acid, hexanoic acid, heptanoic acid, undecanoic acid, lauric acid, benzoic acid, salicylic acid, 2-(4-hydroxybenzoyl)benzoic acid, camphoric acid, cinnamic acid, cyclopentanepropionic acid, digluconic acid, 3-hydroxy-2-naphthoic acid, nicotinic acid, pamoic acid, pectinic acid, 3-phenylpropionic acid, pivalic acid, 2-hydroxyethanesulfonic acid, itaconic acid, trifluoromethanesulfonic acid, dodecylsulfuric acid, ethanesulfonic acid, benzenesulfonic acid, para-toluenesulfonic acid, methanesulfonic acid, 2-naphthalenesulfonic acid, naphthalenedisulfonic acid, camphorsulfonic acid, citric acid, tartaric acid, stearic acid, lactic acid, oxalic acid, malonic acid, succinic acid, malic acid, adipic acid, alginic acid, maleic acid, fumaric acid, D-gluconic acid, mandelic acid, ascorbic acid, glucoheptanoic acid, glycerophosphoric acid, aspartic acid, sulfosalicylic acid or thiocyanic acid, for example.

Further, another suitable pharmaceutically acceptable salt of a sufficiently acidic compound of the present invention is an alkali metal salt, for example a sodium or potassium salt, an alkaline earth metal salt, for example a calcium, magnesium or strontium salt, or an aluminum or zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methylglucamine, N,N-dimethylglucamine, N-ethylglucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognize that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown. Unless specified otherwise, suffixes to chemical names or structural formulae relating to salts, such as “hydrochloride”, “trifluoroacetate”, “sodium salt”, or “x HCl”, “x CF₃COOH”, “x Na+”, for example, mean a salt form, the stoichiometry of this salt not being specified. This applies analogously to cases in which synthesis intermediates or example compounds or salts thereof have been obtained as solvates, for example hydrates, by the preparation and/or purification processes described.

Solvates in the context of the invention are described as those forms of the compounds according to the invention which form a complex in the solid or liquid state by coordination with solvent molecules. Hydrates are a specific form of the solvates in which the coordination is with water. Solvates preferred in the context of the present invention are hydrates.

The compounds of the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof. It is possible to isolate the stereoisomerically homogeneous constituents from such mixtures of enantiomers and/or diastereomers in a known manner. Preference is given to employing chromatographic methods for this purpose, especially HPLC chromatography on achiral or chiral separation phases. In the case of carboxylic acids as intermediates or end products, separation is alternatively also possible via diastereomeric salts using chiral amine bases.

In the context of the present invention, the term “enantiomerically pure” is understood to the effect that the compound in question with respect to the absolute configuration of the chiral centers is present in an enantiomeric excess of more than 95%, preferably more than 98%. The enantiomeric excess, ee, is calculated here by evaluating an HPLC analysis chromatogram on a chiral phase using the formula below:

$$ee = \vert \frac{Enantiomer 1 ( Area percentage ) - Enantiomer 2 ( Area percentage )}{Enantiomer 1 ( Area percentage ) - Enantiomer 2 ( Area percentage )} \vert = \times 100 \% .$$

If the compounds of the invention can occur in tautomeric forms, the present invention encompasses all the tautomeric forms.

The present invention also encompasses all suitable isotopic variants of the compounds of the invention. An isotopic variant of a compound according to the invention is understood here to mean a compound in which at least one atom within the compound according to the invention has been exchanged for another atom of the same atomic number, but with a different atomic mass from the atomic mass which usually or predominantly occurs in nature (“unnatural fraction”). The expression “unnatural fraction” is understood to mean a fraction of such an isotope higher than its natural frequency. The natural frequencies of isotopes to be employed in this connection can be found in “Isotopic Compositions of the Elements 1997”, Pure Appl. Chem., 70(1), 217-235, 1998. Examples of isotopes which can be incorporated into a compound according to the invention are those of hydrogen, carbon, nitrogen, oxygen, phosphorus, sulfur, fluorine, chlorine, bromine and iodine, such as 2H (deuterium), ³H (tritium), ¹³C, ¹⁴C, ¹⁸N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to the comparatively easy preparability and detectability, especially compounds labeled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds of the invention may therefore possibly also constitute a preferred embodiment of the present invention. With regard to the treatment and/or prophylaxis of the disorders specified here, the isotopic variant(s) of the compounds of the general formula (I) preferably contain deuterium (“deuterium-containing compounds of the general formula (I)”). Isotopic variants of the compounds of the general formula (I) into which one or more radioactive isotopes such as ³H or ¹⁴C have been incorporated are beneficial, for example, in medicament and/or substrate tissue distribution studies. Because of their easy incorporability and detectability, these isotopes are particularly preferred. It is possible to incorporate positron-emitting isotopes such as ¹⁸F or ¹¹C into a compound of the general formula (I). These isotopic variants of the compounds of the general formula (I) are suitable for use in in vivo imaging applications. Deuterium-containing and ¹³C-containing compounds of the general formula (I) can be used within the scope of preclinical or clinical studies in mass spectrometry analyses (H. J. Leis et al., Curr. Org. Chem., 1998, 2, 131). Isotopic variants of the compounds of the invention can be prepared by commonly used processes known to those skilled in the art, for example by the methods described further down and the procedures described in the working examples, by using corresponding isotopic modifications of the respective reagents and/or starting compounds.

Isotopic variants of the compounds of the general formula (I) can generally be prepared by processes known to those skilled in the art as described in the schemes and/or examples described here, by replacing a reagent with an isotopic variant of the reagent, preferably a deuterium-containing reagent. According to the deuteration sites desired, it is possible in some cases to incorporate deuterium from D₂O either directly into the compounds or into reagents which can be used for the synthesis of such compounds (Esaki et al., Tetrahedron, 2006, 62, 10954; Esaki et al., Chem. Eur. J., 2007, 13, 4052). A photochemical deuteration and tritiation method has also been described (Y. Y. Loh et al., Science 10.1126/science.aap9674 (2017). Another useful reagent for incorporation of deuterium into molecules is deuterium gas. A rapid route for incorporation of deuterium is the catalytic deuteration of olefinic bonds (H. J. Leis et al., Curr. Org. Chem., 1998, 2, 131; J. R. Morandi et al., J. Org. Chem., 1969, 34 (6), 1889) and acetylenic bonds (N. H. Khan, J. Am. Chem. Soc., 1952, 74 (12), 3018; S. Chandrasekhar et al., Tetrahedron, 2011, 52, 3865). For direct exchange of hydrogen for deuterium in hydrocarbons containing functional groups, it is also possible to use metal catalysts (i.e. Pd, Pt and Rh) in the presence of deuterium gas (J. G. Atkinson et al., U.S. Pat. No. 3,966,781). Various deuterated reagents and synthesis units are commercially available from companies like, for example, C/D/N Isotopes, Quebec, Canada; Cambridge Isotope Laboratories Inc., Andover, MA, USA; and CombiPhos Catalysts, Inc., Princeton, NJ, USA. Further information relating to the prior art with regard to deuterium-hydrogen exchange can be found, for example, in Hanzlik et al., J. Org. Chem., 1990, 55, 3992-3997; R. P. Hanzlik et al., Biochem. Biophys. Res. Commun., 1989, 160, 844; P. J. Reider et al., J. Org. Chem., 1987, 52, 3326-3334; M. Jarman et al., Carcinogenesis, 1993, 16(4), 683-688; J. Atzrodt et al., Angew. Chem., Int. Ed. 2007, 46, 7744; K. Matoishi et al., 2000, J. Chem. Soc, Chem. Commun., 1519-1520; K. Kassahun et al., WO 2012/112363.

The term “deuterium-containing compound of the general formula (I)” is defined as a compound of the general formula (I) in which one or more hydrogen atoms have been replaced by one or more deuterium atoms and in which the frequency of deuterium in every deuterated position in the compound of the general formula (I) is higher than the natural frequency of deuterium, which is about 0.015%. More particularly, in a deuterium-containing compound of the general formula (I), the frequency of deuterium in every deuterated position in the compound of the general formula (I) is higher than 10%, 20%, 30%, 40%, 50%, 60%, 70% or 80%, preferably higher than 90%, 95%, 96% or 97%, even further preferably higher than 98% or 99%, in this position or these positions. It will be apparent that the frequency of deuterium in every deuterated position is independent of the frequency of deuterium in other deuterated positions.

The selective incorporation of one or more deuterium atoms into a compound of the general formula (I) can alter the physicochemical properties (for example acidity [A. Streitwieser et al., J. Am. Chem. Soc., 1963, 85, 2759; C. L. Perrin et al., J. Am. Chem. Soc., 2007, 129, 4490], basicity [C. L. Perrin, et al., J. Am. Chem. Soc., 2003, 125, 15008; C. L. Perrin in Advances in Physical Organic Chemistry, 44, 144; C. L. Perrin et al., J. Am. Chem. Soc., 2005, 127, 9641], lipophilicity [B. Testa et al., Int. J. Pharm., 1984, 19(3), 271]) and/or the metabolic profile of the molecule, and cause changes in the ratio of parent compound to metabolites or the amounts of metabolites formed. Such changes may lead to particular therapeutic benefits and therefore be preferable under particular circumstances. Reduced rates of metabolism and metabolic switching, where the ratio of metabolites is changed, have been reported (D. J. Kushner et al., Can. J. Physiol. Pharmacol., 1999, 77, 79; A. E. Mutlib et al., Toxicol. Appl. Pharmacol., 2000, 169, 102). These changes in the exposure to parent compound and metabolites can have important consequences with respect to the pharmacodynamics, tolerability and efficacy of a deuterium-containing compound of the general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Uetrecht et al., Chemical Research in Toxicology, 2008, 21, 9, 1862; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. Indiplon (A. J. Morales et al., Abstract 285, The 15^{th} North American Meeting of the International Society of Xenobiotics, San Diego, CA, Oct. 12-16, 2008), ML-337 (C. J. Wenthur et al., J. Med. Chem., 2013, 56, 5208), and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch. Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

The present invention additionally also encompasses prodrugs of the compounds of the invention. The term “prodrugs” refers here to compounds which may themselves be biologically active or inactive, but are converted while present in the body, for example by a metabolic or hydrolytic route, to compounds of the invention.

In the context of the present invention, unless specified otherwise, the substituents are defined as follows:

Alkyl in the context of the invention is a straight-chain or branched alkyl radical having the particular number of carbon atoms specified. Examples include: methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl, 2-ethylbutyl, 1,4-dimethylpentyl, 4,4-dimethylpentyl and 1,4,4-trimethylpentyl.

Alkoxy in the context of the invention is a straight-chain or branched alkoxy radical having 1 to 4 carbon atoms. Examples include: methoxy, ethoxy, n-propoxy, isopropoxy, 1-methylpropoxy, n-butoxy, isobutoxy and tert-butoxy.

Cycloalkoxy in the context of the invention is a cyclic alkoxy radical having 3 to 4 carbon atoms. Examples include: cyclopropoxy or cyclobutoxy.

Cycloalkyl or carbocycle in the context of the invention is a mono-, poly- or spirocyclic, preferably mono- or bicyclic, saturated carbocycle having a total of 3 to 8 ring atoms. A monocyclic saturated carbocycle is referred to synonymously as cycloalkyl. Examples include: cyclopropyl, cyclobutyl, cyclopentyl, cyclohexyl, cycloheptyl, cyclopentenyl, cyclohexenyl, cyclohexadienyl, cycloheptenyl, cycloheptadienyl, spiro[2.3]hexyl, spiro[2.4]heptyl, spiro[2.5]octyl, bicyclo[1.1.1]pentyl, bicyclo[2.2.1]heptyl, bicyclo[4.1.0]heptyl, bicyclo[2.2.2]octyl, tricyclo[3.3.1.13,7]decyl. Monocyclic cycloalkyl having 3 to 5 carbon atoms is preferred. Examples include: cyclopropyl, cyclobutyl or cyclopentyl.

5- or 6-membered heteroaryl in the context of the invention is a monocyclic aromatic heterocycle (heteroaromatic) which has a total of 5 or 6 ring atoms, contains up to three identical or different ring heteroatoms from the series N, O and/or S and is attached via a ring carbon atom or optionally via a ring nitrogen atom. Examples include: furyl, pyrrolyl, thienyl, pyrazolyl, imidazolyl, thiazolyl, oxazolyl, isoxazolyl, isothiazolyl, triazolyl, oxadiazolyl, thiadiazolyl, tetrazolyl, pyridyl, pyrimidinyl, pyridazinyl or pyrazinyl.

In general, and unless stated otherwise, the heteroaryl groups include all possible isomeric forms, for example tautomers and positional isomers in relation to the attachment point to the rest of the molecule. Thus, the term pyridyl embraces, as non-limiting examples, 2-pyridyl, 3-pyridyl and 4-pyridyl, or the term thienyl embraces 2-thienyl and 3-thienyl.

Halogen in the context of the invention includes fluorine, chlorine, bromine and iodine. Preference is given to chlorine or fluorine.

When radicals in the compounds of the invention are substituted, the radicals may be mono- or polysubstituted, unless specified otherwise. In the context of the present invention, all radicals which occur more than once are defined independently of one another. When radicals in the compounds of the invention are substituted, the radicals may be mono- or polysubstituted, unless specified otherwise. Substitution by one substituent or by two identical or different substituents is preferred.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

A further embodiment of the present invention relates to combinations of compounds of formula (I)

in which
X represents S or N;
Y represents N, S or O,
   where, if X represents S, then Y represents N;
Z represents CR₄, N or O,
   where, if X represents N and Y represents N, then Z represents 0;
   where, if X represents S, then Z represents N or CR₄
R₁ represents pyridinyl, pyrazolyl, thiazolyl, thienyl, phenyl,
   where pyridinyl may be substituted by 1 to 2 substituents independently of one another selected from the group of (C₁-C₂)-alkyl, fluorine, chlorine, trifluoromethyl, trifluoromethoxy,
   where pyrazolyl may be substituted by 1 to 2 substituents independently of one another selected from the group of (C₁-C₂)-alkyl, fluorine, chlorine, trifluoromethyl,
   where thiazolyl may be substituted by 1 to 2 substituents independently of one another selected from the group of fluorine, chlorine,
   where thienyl may be substituted by 1 to 2 substituents independently of one another selected from the group of fluorine, chlorine,
   where phenyl may be substituted by 1 to 2 substituents independently of one another selected from the group of (C₁-C₂)-alkyl, (C₃-C₄)-cycloalkyl, methoxy, cyano, hydroxy, fluorine, chlorine, trifluoromethyl;
R₂ represents hydrogen, (C₁-C₂)-alkyl, or
   together with the carbon atom to which R₂ is attached forms a cyclopropyl ring,
R₃ represents hydrogen, (C₁-C₂)-alkyl;
R₄ represents hydrogen, (C₁-C₂)-alkyl, (C₃-C₄)-cycloalkyl, trifluoromethyl, bromine, chlorine, phenyl; where phenyl may be substituted by halogen,
R₅ represents hydrogen, (C₁-C₂)-alkyl, methoxy, fluorine;
R₆ represents a group of the formula a), b), c) or e),

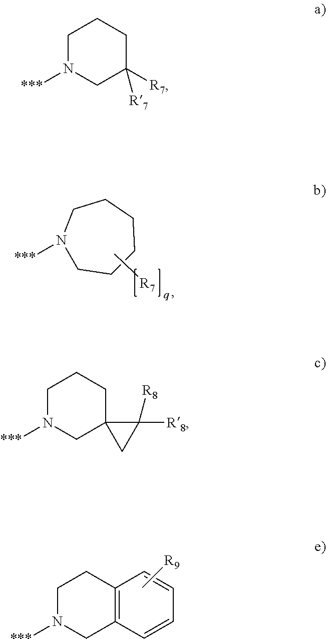

where *** marks the attachment to the adjacent piperidine ring,
where R₇ or R′₇ independently of one another represent hydrogen, (C₁-C₄)-alkyl, (C₃-C₄)-cycloalkyl, (C₁-C₂)-alkoxy, (C₃-C₄)-cycloalkoxy, monofluoromethyl, difluoromethyl, trifluoromethyl, difluoromethoxy, phenyl,
   where (C₁-C₄)-alkyl may be substituted by methoxy, n-butoxy, cyclopropyl, cyclobutoxy and up to disubstituted by fluorine,
      where methoxy may be substituted by cyclopropyl, cyclobutyl, trifluoromethyl,
      where cyclopropyl may be substituted by monofluoromethyl, difluoromethyl, trifluoromethyl,
      where cyclobutyl may be up to disubstituted by fluorine,
      where n-butoxy may be up to disubstituted by fluorine,
   where (C₁-C₂)-alkoxy may be substituted by cyclopropyl, cyclobutyl, cyclobutoxy, trifluoromethyl and
      where cyclopropyl and cyclobutyl may be up to disubstituted by fluorine,
   where (C₃-C₄)-cycloalkoxy may be up to disubstituted by fluorine,
where R₈ or R′₈ independently of one another represent hydrogen or fluorine,
where R₉ represents hydrogen, (C₁-C₄)-alkyl, (C₁-C₂)-alkoxy, methoxyethyl, fluorine, chlorine;
n represents 0 or 1 and
m represents 1 or 2,
q represents 0 or 2, and
muscarinic receptor antagonists,
and the salts, solvates and solvates of the salts thereof.

A further embodiment of the present invention relates to combinations of compounds of formula (I)

in which
X represents S or N;
Y represents N, S or O,
   where, if X represents S, then Y represents N;
Z represents CR₄, N or O,
   where, if X represents N and Y represents N, then Z represents 0;
   where, if X represents S, then Z represents N or CR₄
R₁ represents pyridinyl, pyrazolyl, thiazolyl, thienyl, phenyl,
   where pyridinyl may be substituted by 1 to 2 substituents independently of one another selected from the group of (C₁-C₂)-alkyl, fluorine, chlorine, trifluoromethyl, trifluoromethoxy,
   where pyrazolyl may be substituted by 1 to 2 substituents independently of one another selected from the group of (C₁-C₂)-alkyl, fluorine, chlorine, trifluoromethyl,
   where thiazolyl may be substituted by 1 to 2 substituents independently of one another selected from the group of fluorine, chlorine,
   where thienyl may be substituted by 1 to 2 substituents independently of one another selected from the group of fluorine, chlorine,
   where phenyl may be substituted by 1 to 2 substituents independently of one another selected from the group of (C₁-C₂)-alkyl, (C₃-C₄)-cycloalkyl, methoxy, cyano, hydroxy, fluorine, chlorine, trifluoromethyl;
R₂ represents hydrogen, (C₁-C₂)-alkyl, or
   together with the carbon atom to which R₂ is attached forms a cyclopropyl ring,
R₃ represents hydrogen, (C₁-C₂)-alkyl;
R₄ represents hydrogen, (C₁-C₂)-alkyl, (C₃-C₄)-cycloalkyl, trifluoromethyl, bromine, chlorine, phenyl;
   where phenyl may be substituted by halogen,
R₅ represents hydrogen, (C₁-C₂)-alkyl, methoxy, fluorine;
R₆ represents a group of the formula a), b), c) or e),

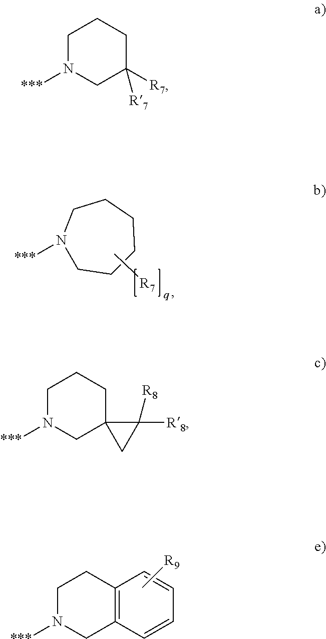

where *** marks the attachment to the adjacent piperidine ring,
where R₇ or R′₇ independently of one another represent hydrogen, (C₁-C₄)-alkyl, (C₃-C₄)-cycloalkyl, (C₁-C₂)-alkoxy, (C₃-C₄)-cycloalkoxy, monofluoromethyl, difluoromethyl, trifluoromethyl, difluoromethoxy, phenyl,
   where (C₁-C₄)-alkyl may be substituted by methoxy, n-butoxy, cyclopropyl, cyclobutoxy and up to disubstituted by fluorine,
      where methoxy may be substituted by cyclopropyl, cyclobutyl, trifluoromethyl,
      where cyclopropyl may be substituted by monofluoromethyl, difluoromethyl, trifluoromethyl,
      where cyclobutyl may be up to disubstituted by fluorine,
      where n-butoxy may be up to disubstituted by fluorine,
   where (C₁-C₂)-alkoxy may be substituted by cyclopropyl, cyclobutyl, cyclobutoxy, trifluoromethyl and
      where cyclopropyl and cyclobutyl may be up to disubstituted by fluorine,
   where (C₃-C₄)-cycloalkoxy may be up to disubstituted by fluorine,
where R₈ or R′₈ independently of one another represent hydrogen or fluorine,
where R₉ represents hydrogen, (C₁-C₄)-alkyl, (C₁-C₂)-alkoxy, methoxyethyl, fluorine, chlorine;
n represents 0 or 1 and
m represents 1 or 2,
q represents 0 or 2, and
muscarinic receptor antagonists selected from the group comprising Oxybutynin, R-Oxybutynin and Tolterodine,
and the salts, solvates and solvates of the salts thereof.

A further embodiment of the present invention relates to combinations of compounds of formula (I)

in which
X, Y and Z are selected such that the aromatic 5-membered ring has the structural formula h), i), j), k) or (r),

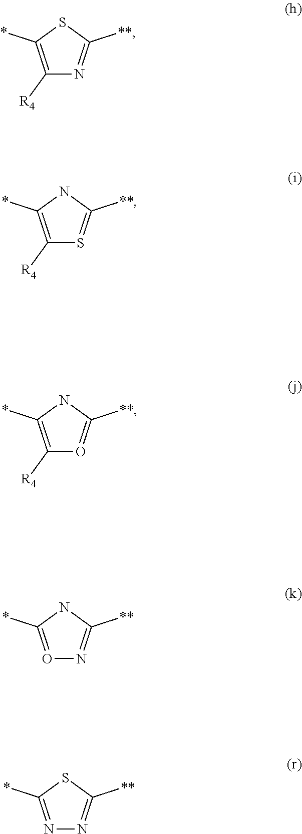

where * marks the attachment to the carbonyl group and ** marks the attachment to the nitrogen atom of the adjacent piperidine ring and
R₁ represents pyridinyl, pyrazolyl, thiazolyl, thienyl, phenyl,
   where pyridinyl may be substituted by 1 to 2 substituents independently of one another selected from the group of (C₁-C₂)-alkyl, fluorine, chlorine, trifluoromethyl, trifluoromethoxy,
   where pyrazolyl may be substituted by 1 to 2 substituents independently of one another selected from the group of (C₁-C₂)-alkyl, fluorine, chlorine, trifluoromethyl,
   where thiazolyl may be substituted by chlorine,
   where thienyl may be substituted by fluorine,
   where phenyl may be substituted by 1 to 2 substituents independently of one another selected from the group of (C₁-C₂)-alkyl, (C₃-C₄)-cycloalkyl, methoxy, cyano, hydroxy, fluorine, chlorine, trifluoromethyl;
R₂ represents hydrogen, methyl, or
   together with the carbon atom to which R₂ is attached forms a cyclopropyl ring,
R₃ represents hydrogen, (C₁-C₂)-alkyl;
R₄ represents hydrogen, methyl, ethyl, cyclopropyl, trifluoromethyl, bromine, chlorine, phenyl;
   where phenyl may be substituted by chlorine,
R₅ represents hydrogen, fluorine;
R₆ represents a group of the formula a), b′), b″), c′), c″) or e),

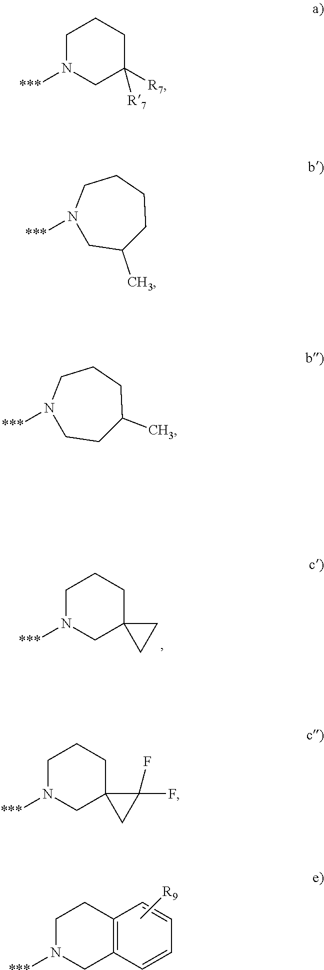

where *** marks the attachment to the adjacent piperidine ring,
where R₇ or R′₇ independently of one another represent hydrogen, (C₁-C₄)-alkyl, (C₃-C₄)-cycloalkyl, (C₁-C₂)-alkoxy, (C₃-C₄)-cycloalkoxy, monofluoromethyl, difluoromethyl, trifluoromethyl, difluoromethoxy, phenyl,
   where (C₁-C₄)-alkyl may be substituted by methoxy, n-butoxy, cyclopropyl, cyclobutoxy and up to disubstituted by fluorine,
      where methoxy may be substituted by cyclopropyl, cyclobutyl, trifluoromethyl,
      where cyclopropyl may be substituted by monofluoromethyl, difluoromethyl, trifluoromethyl,
      where cyclobutyl may be up to disubstituted by fluorine,
      where n-butoxy may be up to disubstituted by fluorine,
   where (C₁-C₂)-alkoxy may be substituted by cyclopropyl, cyclobutyl, cyclobutoxy, trifluoromethyl and
      where cyclopropyl and cyclobutyl may be up to disubstituted by fluorine,
   where (C₃-C₄)-cycloalkoxy may be up to disubstituted by fluorine,
where R₉ represents hydrogen, methyl, tert-butyl, methoxy, methoxymethyl, fluorine, chlorine;
n represents 0 or 1 and
m represents 1 or 2, and
muscarinic receptor antagonists,
and the salts, solvates and solvates of the salts thereof.

A further embodiment of the present invention relates to combinations of compounds of formula (I)

in which
X, Y and Z are selected such that the aromatic 5-membered ring has the structural formula h), i), j), k) or (r),

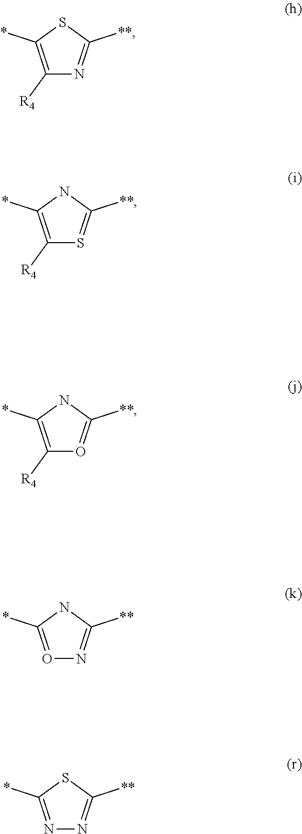

where * marks the attachment to the carbonyl group and ** marks the attachment to the nitrogen atom of the
adjacent piperidine ring and R₁ represents pyridinyl, pyrazolyl, thiazolyl, thienyl, phenyl,
   where pyridinyl may be substituted by 1 to 2 substituents independently of one another selected from the group of (C₁-C₂)-alkyl, fluorine, chlorine, trifluoromethyl, trifluoromethoxy,
   where pyrazolyl may be substituted by 1 to 2 substituents independently of one another selected from the group of (C₁-C₂)-alkyl, fluorine, chlorine, trifluoromethyl,
   where thiazolyl may be substituted by chlorine,
   where thienyl may be substituted by fluorine,
   where phenyl may be substituted by 1 to 2 substituents independently of one another selected from the group of (C₁-C₂)-alkyl, (C₃-C₄)-cycloalkyl, methoxy, cyano, hydroxy, fluorine, chlorine, trifluoromethyl;
R₂ represents hydrogen, methyl, or
   together with the carbon atom to which R₂ is attached forms a cyclopropyl ring,
R₃ represents hydrogen, (C₁-C₂)-alkyl;
R₄ represents hydrogen, methyl, ethyl, cyclopropyl, trifluoromethyl, bromine, chlorine, phenyl; where phenyl may be substituted by chlorine,
R₅ represents hydrogen, fluorine;
R₆ represents a group of the formula a), b′), b″), c′), c″) or e),

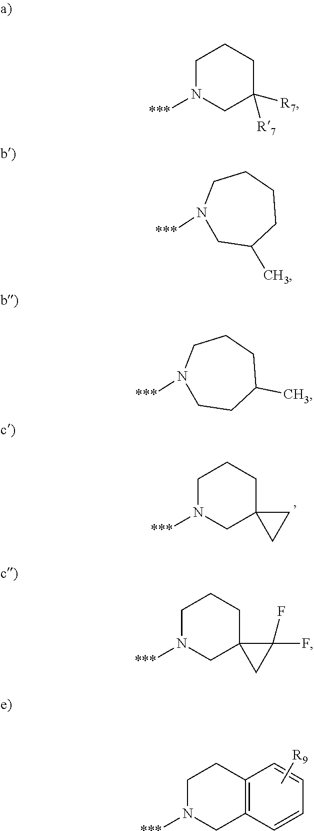

where *** marks the attachment to the adjacent piperidine ring,
where R₇ or R′₇ independently of one another represent hydrogen, (C₁-C₄)-alkyl, (C₃-C₄)-cycloalkyl, (C₁-C₂)-alkoxy, (C₃-C₄)-cycloalkoxy, monofluoromethyl, difluoromethyl, trifluoromethyl, difluoromethoxy, phenyl,
   where (C₁-C₄)-alkyl may be substituted by methoxy, n-butoxy, cyclopropyl, cyclobutoxy and up to disubstituted by fluorine,
      where methoxy may be substituted by cyclopropyl, cyclobutyl, trifluoromethyl,
      where cyclopropyl may be substituted by monofluoromethyl, difluoromethyl, trifluoromethyl,
      where cyclobutyl may be up to disubstituted by fluorine,
      where n-butoxy may be up to disubstituted by fluorine,
   where (C₁-C₂)-alkoxy may be substituted by cyclopropyl, cyclobutyl, cyclobutoxy, trifluoromethyl and
      where cyclopropyl and cyclobutyl may be up to disubstituted by fluorine,
   where (C₃-C₄)-cycloalkoxy may be up to disubstituted by fluorine,
where R₉ represents hydrogen, methyl, tert-butyl, methoxy, methoxymethyl, fluorine, chlorine;
n represents 0 or 1 and
m represents 1 or 2, and
muscarinic receptor antagonists selected from the group comprising Oxybutynin, R-Oxybutynin and Tolterodine,
and the salts, solvates and solvates of the salts thereof.

A further embodiment of the present invention relates to combinations of compounds of formula (I)

in which
X, Y and Z are selected such that the aromatic 5-membered ring has the structural formula h), i), j), k) or (r),

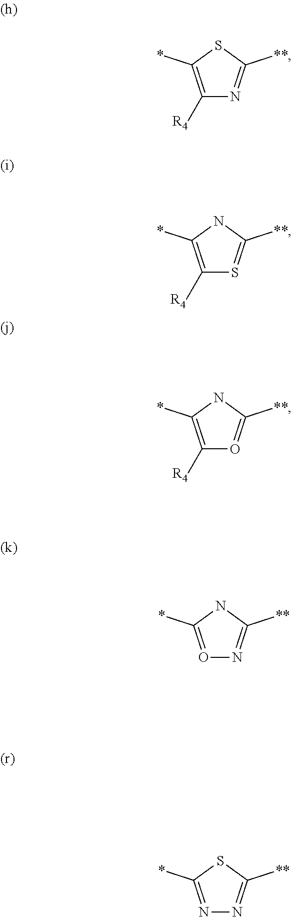

where * marks the attachment to the carbonyl group and ** marks the attachment to the nitrogen atom of the adjacent piperidine ring and
R₁ represents pyridinyl, pyrazolyl, thiazolyl, thienyl, phenyl,
   where pyridinyl may be substituted by 1 to 2 substituents independently of one another selected from the group of (C₁-C₂)-alkyl, fluorine, chlorine, trifluoromethyl, trifluoromethoxy,
   where pyrazolyl may be substituted by 1 to 2 substituents independently of one another selected from the group of (C₁-C₂)-alkyl, fluorine, chlorine, trifluoromethyl,
   where thiazolyl may be substituted by chlorine,
   where thienyl may be substituted by fluorine,
   where phenyl may be substituted by 1 to 2 substituents independently of one another selected from the group of (C₁-C₂)-alkyl, (C₃-C₄)-cycloalkyl, methoxy, cyano, hydroxy, fluorine, chlorine, trifluoromethyl;
R₂ represents hydrogen, methyl, or
   together with the carbon atom to which R₂ is attached forms a cyclopropyl ring,
R₃ represents hydrogen, (C₁-C₂)-alkyl;
R₄ represents hydrogen, methyl, ethyl, cyclopropyl, trifluoromethyl, bromine, chlorine, phenyl;
   where phenyl may be substituted by chlorine,
R₅ represents hydrogen, fluorine;
R₆ represents a group of the formula a), b′), b″), c′), c″) or e),

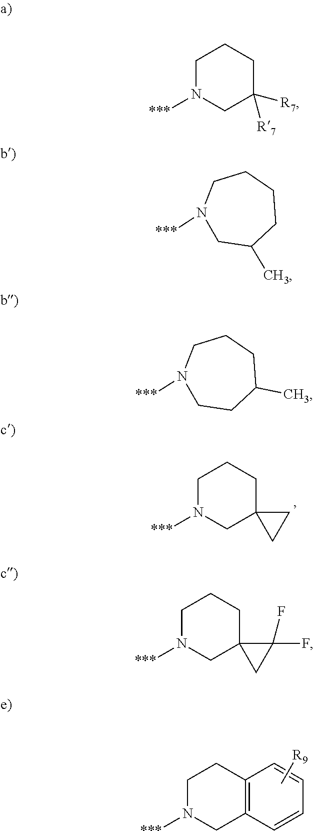

where *** marks the attachment to the adjacent piperidine ring,
where R₇ or R′₇ independently of one another represent hydrogen, (C₁-C₄)-alkyl, (C₃-C₄)-cycloalkyl, (C₁-C₂)-alkoxy, (C₃-C₄)-cycloalkoxy, monofluoromethyl, difluoromethyl, trifluoromethyl, difluoromethoxy, phenyl,
   where (C₁-C₄)-alkyl may be substituted by methoxy, n-butoxy, cyclopropyl, cyclobutoxy and up to disubstituted by fluorine,
      where methoxy may be substituted by cyclopropyl, cyclobutyl, trifluoromethyl,
      where cyclopropyl may be substituted by monofluoromethyl, difluoromethyl, trifluoromethyl,
      where cyclobutyl may be up to disubstituted by fluorine,
      where n-butoxy may be up to disubstituted by fluorine,
   where (C₁-C₂)-alkoxy may be substituted by cyclopropyl, cyclobutyl, cyclobutoxy, trifluoromethyl and
      where cyclopropyl and cyclobutyl may be up to disubstituted by fluorine,
   where (C₃-C₄)-cycloalkoxy may be up to disubstituted by fluorine,
where R₉ represents hydrogen, methyl, tert-butyl, methoxy, methoxymethyl, fluorine, chlorine;
n represents 0 or 1 and
m represents 1 or 2, and
Oxybutynin,
and the salts, solvates and solvates of the salts thereof.

A further embodiment of the present invention relates to combinations of compounds of formula (I)

in which
X, Y and Z are selected such that the aromatic 5-membered ring has the structural formula h), i), j), k) or (r),

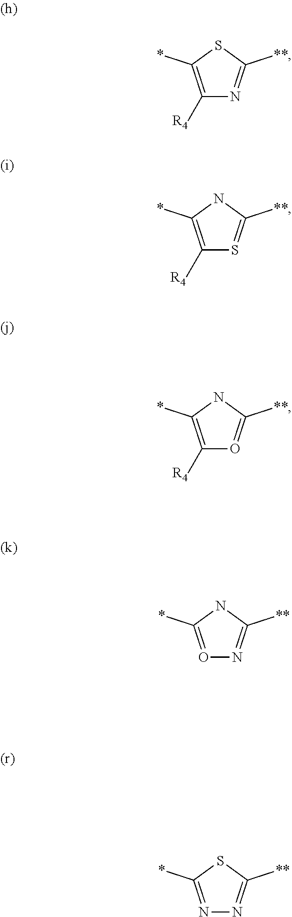

where * marks the attachment to the carbonyl group and ** marks the attachment to the nitrogen atom of the adjacent piperidine ring and
R₁ represents pyridinyl, pyrazolyl, thiazolyl, thienyl, phenyl,
   where pyridinyl may be substituted by 1 to 2 substituents independently of one another selected from the group of (C₁-C₂)-alkyl, fluorine, chlorine, trifluoromethyl, trifluoromethoxy,
   where pyrazolyl may be substituted by 1 to 2 substituents independently of one another selected from the group of (C₁-C₂)-alkyl, fluorine, chlorine, trifluoromethyl,
   where thiazolyl may be substituted by chlorine,
   where thienyl may be substituted by fluorine,
   where phenyl may be substituted by 1 to 2 substituents independently of one another selected from the group of (C₁-C₂)-alkyl, (C₃-C₄)-cycloalkyl, methoxy, cyano, hydroxy, fluorine, chlorine, trifluoromethyl;
R₂ represents hydrogen, methyl, or
   together with the carbon atom to which R₂ is attached forms a cyclopropyl ring,
R₃ represents hydrogen, (C₁-C₂)-alkyl;
R₄ represents hydrogen, methyl, ethyl, cyclopropyl, trifluoromethyl, bromine, chlorine, phenyl;
   where phenyl may be substituted by chlorine,
R₅ represents hydrogen, fluorine;
R₆ represents a group of the formula a), b′), b″), c′), c″) or e),

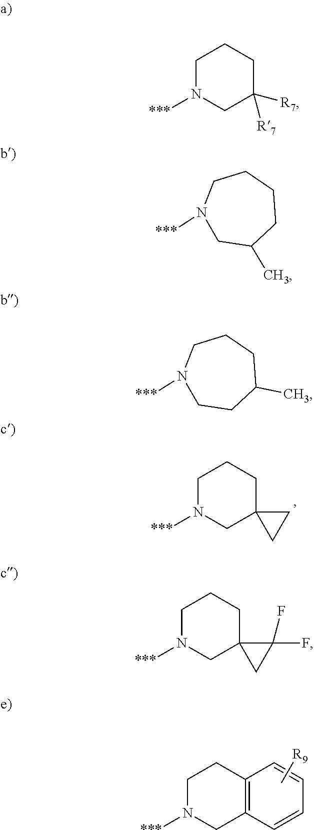

where *** marks the attachment to the adjacent piperidine ring,
where R₇ or R′₇ independently of one another represent hydrogen, (C₁-C₄)-alkyl, (C₃-C₄)-cycloalkyl, (C₁-C₂)-alkoxy, (C₃-C₄)-cycloalkoxy, monofluoromethyl, difluoromethyl, trifluoromethyl, difluoromethoxy, phenyl,
   where (C₁-C₄)-alkyl may be substituted by methoxy, n-butoxy, cyclopropyl, cyclobutoxy and up to disubstituted by fluorine,
      where methoxy may be substituted by cyclopropyl, cyclobutyl, trifluoromethyl,
      where cyclopropyl may be substituted by monofluoromethyl, difluoromethyl, trifluoromethyl,
      where cyclobutyl may be up to disubstituted by fluorine,
      where n-butoxy may be up to disubstituted by fluorine,
   where (C₁-C₂)-alkoxy may be substituted by cyclopropyl, cyclobutyl, cyclobutoxy, trifluoromethyl and
      where cyclopropyl and cyclobutyl may be up to disubstituted by fluorine,
   where (C₃-C₄)-cycloalkoxy may be up to disubstituted by fluorine,
where R₉ represents hydrogen, methyl, tert-butyl, methoxy, methoxymethyl, fluorine, chlorine;
n represents 0 or 1 and
m represents 1 or 2, and
R-Oxybutynin,
and the salts, solvates and solvates of the salts thereof.

A further embodiment of the present invention relates to combinations of compounds of formula (I)

in which
X, Y and Z are selected from the group of S, N, O and C to form 1,3-thiazolyl, 1,3-oxazolyl or 1,2,4-oxadiazolyl,
R₁ represent pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R₃ represents hydrogen or methyl;
R₄ represents hydrogen, methyl, ethyl or trifluormethyl;
R₅ represents hydrogen or fluoro;
R₆ represents a group of the formula a), c′) or c″),

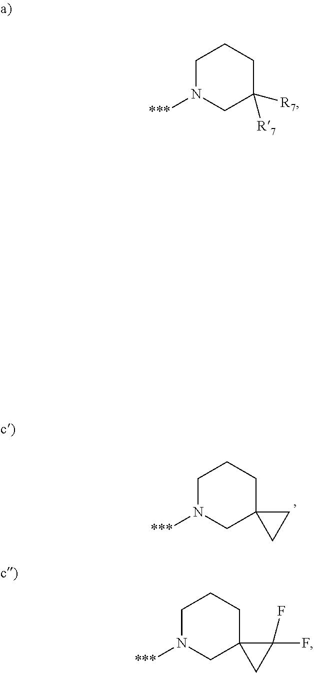

in which *** marks the bond to the adjacent piperidine ring,
wherein R₇ or R′₇ independently from each other represent hydrogen, methyl, ethyl, n-propyl, iso-propyl, tert-butyl, 2-fluoroethyl, cyclopropyl, cyclobutyl, cyclopropylmethyl, methoxy, ethoxy, methoxymethyl, monofluoromethyl, difluoromethyl, trifluormethyl, difluormethoxy, 3,3-difluorocyclobutylmethoxy, cyclobutylmethoxy, cyclopropylmethoxy, cyclopropyl-methoxymethyl, cyclobutyloxymethyl, 3-fluorobutyloxymethyl, 3,3-difluorocyclobutyl-methoxymethyl, 2,2,2-trifluoroethoxy, 2,2,2-trifluoroethoxymethyl, 2,2-difluorocyclopropyl-methoxy, cyclobutyloxy, 3,3-difluorocyclobutyloxy, fluoromethylcyclopropylmethoxy, difluoromethylcyclopropylmethoxy, trifluoromethylcyclopropylmethoxy or fluoro;
n represents 0 or 1,
m represents 1, and
muscarinic receptor antagonists,
and the salts, solvates and solvates of the salts thereof.

A further embodiment of the present invention relates to combinations of compounds of formula (I)

in which
X, Y and Z are selected from the group of S, N, O and C to form 1,3-thiazolyl, 1,3-oxazolyl, or 1,2,4-oxadiazolyl;
R₁ represent pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R₃ represents hydrogen or methyl;
R₄ represents hydrogen, methyl, ethyl or trifluormethyl;
   wherein phenyl may in turn be substituted by chloro,
R₅ represents hydrogen or fluoro,
R₆ represents a group of the formula a), c′) or c″),

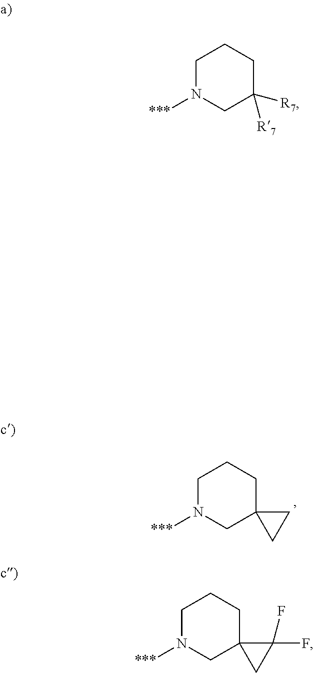

in which *** marks the bond to the adjacent piperidine ring,
wherein R₇ or R′₇ independently from each other represent hydrogen, methyl, ethyl, n-propyl, iso-propyl, tert-butyl, 2-fluoroethyl, cyclopropyl, cyclobutyl, cyclopropylmethyl, methoxy, ethoxy, methoxymethyl, monofluoromethyl, difluoromethyl, trifluormethyl, difluormethoxy, 3,3-difluorocyclobutylmethoxy, cyclobutylmethoxy, cyclopropylmethoxy, cyclopropyl-methoxymethyl, cyclobutyloxymethyl, 3-fluorobutyloxymethyl, 3,3-difluorocyclobutyl-methoxymethyl, 2,2,2-trifluoroethoxy, 2,2,2-trifluoroethoxymethyl, 2,2-difluorocyclopropyl-methoxy, cyclobutyloxy, 3,3-difluorocyclobutyloxy, fluoromethylcyclopropylmethoxy, difluoromethylcyclopropylmethoxy, trifluoromethylcyclopropylmethoxy or fluoro;
n represents 0 or 1,
m represents 1, and
muscarinic receptor antagonists selected from the group comprising Oxybutynin, R-Oxybutynin and Tolterodine,
and the salts, solvates and solvates of the salts thereof.

A further embodiment of the present invention relates to combinations of compounds of formula (I)

in which
X, Y and Z are selected from the group of S, N, O and C to form 1,3-thiazolyl, 1,3-oxazolyl or 1,2,4-oxadiazolyl,
R₁ represent pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R₃ represents hydrogen or methyl;
R₄ represents hydrogen, methyl, ethyl or trifluormethyl;
   wherein phenyl may in turn be substituted by chloro,
R₅ represents hydrogen or fluoro;
R₆ represents a group of the formula a), c′) or c″),

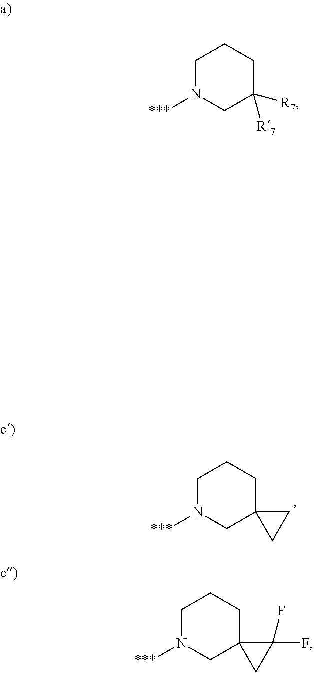

in which *** marks the bond to the adjacent piperidine ring,
wherein R₇ or R′₇ independently from each other represent hydrogen, methyl, ethyl, n-propyl, iso-propyl, tert-butyl, 2-fluoroethyl, cyclopropyl, cyclobutyl, cyclopropylmethyl, methoxy, ethoxy, methoxymethyl, monofluoromethyl, difluoromethyl, trifluormethyl, difluormethoxy, 3,3-difluorocyclobutylmethoxy, cyclobutylmethoxy, cyclopropylmethoxy, cyclopropyl-methoxymethyl, cyclobutyloxymethyl, 3-fluorobutyloxymethyl, 3,3-difluorocyclobutyl-methoxymethyl, 2,2,2-trifluoroethoxy, 2,2,2-trifluoroethoxymethyl, 2,2-difluorocyclopropyl-methoxy, cyclobutyloxy, 3,3-difluorocyclobutyloxy, fluoromethylcyclopropylmethoxy, difluoromethylcyclopropylmethoxy, trifluoromethylcyclopropylmethoxy or fluoro;
n represents 0 or 1,
m represents 1, and
Oxybutynin,
and the salts, solvates and solvates of the salts thereof.

A further embodiment of the present invention relates to combinations of compounds of formula (I)

in which
X, Y and Z are selected from the group of S, N, O and C to form 1,3-thiazolyl, 1,3-oxazolyl, or 1,2,4-oxadiazolyl;
R₁ represent pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R₃ represents hydrogen or methyl;
R₄ represents hydrogen, methyl, ethyl or trifluormethyl;
   wherein phenyl may in turn be substituted by chloro,
R₅ represents hydrogen or fluoro,
R₆ represents a group of the formula a), c′) or c″),

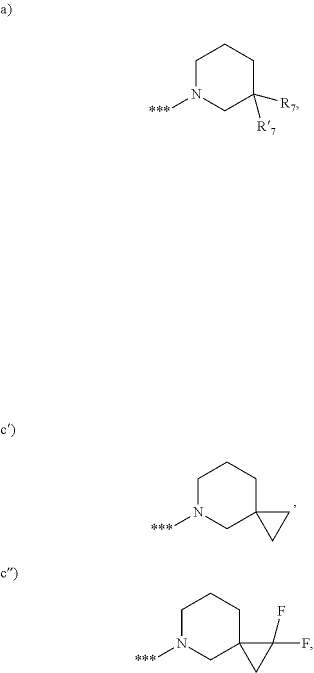

in which *** marks the bond to the adjacent piperidine ring,
wherein R₇ or R′₇ independently from each other represent hydrogen, methyl, ethyl, n-propyl, iso-propyl, tert-butyl, 2-fluoroethyl, cyclopropyl, cyclobutyl, cyclopropylmethyl, methoxy, ethoxy, methoxymethyl, monofluoromethyl, difluoromethyl, trifluormethyl, difluormethoxy, 3,3-difluorocyclobutylmethoxy, cyclobutylmethoxy, cyclopropylmethoxy, cyclopropyl-methoxymethyl, cyclobutyloxymethyl, 3-fluorobutyloxymethyl, 3,3-difluorocyclobutyl-methoxymethyl, 2,2,2-trifluoroethoxy, 2,2,2-trifluoroethoxymethyl, 2,2-difluorocyclopropyl-methoxy, cyclobutyloxy, 3,3-difluorocyclobutyloxy, fluoromethylcyclopropylmethoxy, difluoromethylcyclopropylmethoxy, trifluoromethylcyclopropylmethoxy or fluoro;
n represents 0 or 1,
m represents 1, and
R-Oxybutynin,
and the salts, solvates and solvates of the salts thereof.

A further embodiment of the present invention relates to combinations of compounds of formula (I)

in which
X, Y and Z are selected from the group of S, N, O and C to form 1,3-thiazolyl, 1,3-oxazolyl, or 1,2,4-oxadiazolyl;
R₁ represent pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R₃ represents hydrogen or methyl;
R₄ represents hydrogen, methyl, ethyl or trifluormethyl;
   wherein phenyl may in turn be substituted by chloro,
R₅ represents hydrogen or fluoro,
R₆ represents a group of the formula a), c′) or c″),

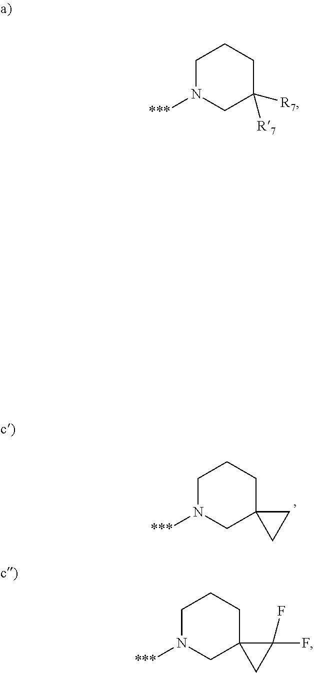

in which *** marks the bond to the adjacent piperidine ring,
wherein R₇ or R′₇ independently from each other represent hydrogen, methyl, ethyl, n-propyl, iso-propyl, tert-butyl, 2-fluoroethyl, cyclopropyl, cyclobutyl, cyclopropylmethyl, methoxy, ethoxy, methoxymethyl, monofluoromethyl, difluoromethyl, trifluormethyl, difluormethoxy, 3,3-difluorocyclobutylmethoxy, cyclobutylmethoxy, cyclopropylmethoxy, cyclopropyl-methoxymethyl, cyclobutyloxymethyl, 3-fluorobutyloxymethyl, 3,3-difluorocyclobutyl-methoxymethyl, 2,2,2-trifluoroethoxy, 2,2,2-trifluoroethoxymethyl, 2,2-difluorocyclopropyl-methoxy, cyclobutyloxy, 3,3-difluorocyclobutyloxy, fluoromethylcyclopropylmethoxy, difluoromethylcyclopropylmethoxy, trifluoromethylcyclopropylmethoxy or fluoro;
n represents 0 or 1,
m represents 1, and
Tolterodine,
and the salts, solvates and solvates of the salts thereof.

A further embodiment of the present invention relates to combinations of compounds of formula (I)

in which
X, Y and Z are selected such that the aromatic 5-membered ring has the structural formula h′)

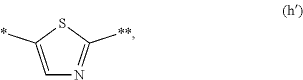

R₁ represents pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl, 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R₃ represents hydrogen;
R₅ represents hydrogen, fluorine;
R₆ represents a group of the formula a), c′) or c″)

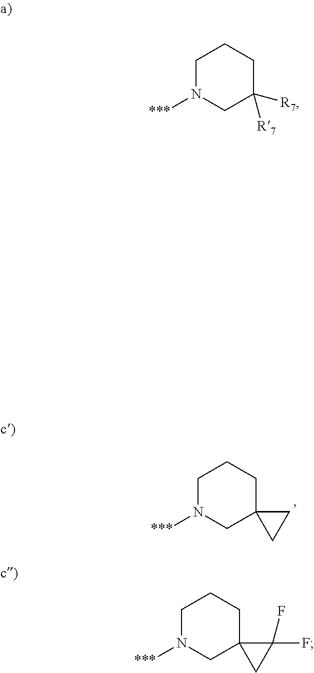

where *** marks the attachment to the adjacent piperidine ring,
where R₇ and R′₇ independently of one another represent hydrogen, methyl, ethyl, n-propyl, isopropyl, tert-butyl, 2-fluoroethyl, cyclopropyl, cyclobutyl, cyclopropylmethyl, methoxy, ethoxy, methoxymethyl, monofluoromethyl, difluoromethyl, trifluoromethyl, difluoromethoxy, 3,3-difluorocyclobutylmethoxy, cyclobutylmethoxy, cyclopropylmethoxy, cyclopropylmethoxymethyl, cyclobutyloxymethyl, 3-fluorobutyloxymethyl, 3,3-difluorocyclobutylmethoxymethyl, 2,2,2-trifluoroethoxy, 2,2,2-trifluoroethoxymethyl, 2,2-difluorocyclopropylmethoxy, cyclobutyloxy, 3,3-difluorocyclobutyloxy, fluoromethylcyclopropylmethoxy, difluoromethylcyclopropylmethoxy, trifluoromethylcyclopropylmethoxy, fluorine;
n represents 0 or 1 and
m represents 1, and
muscarinic receptor antagonists,
and the salts, solvates and solvates of the salts thereof.

A further embodiment of the present invention relates to combinations of compounds of formula (I)

in which
X, Y and Z are selected such that the aromatic 5-membered ring has the structural formula h′)

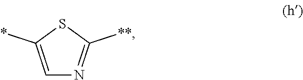

R₁ represents pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl, 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R₃ represents hydrogen;
R₅ represents hydrogen, fluorine;
R₆ represents a group of the formula a), c′) or c″)

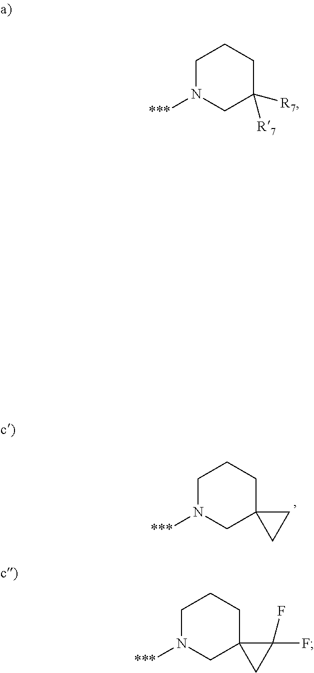

where *** marks the attachment to the adjacent piperidine ring,
where R₇ and R′₇ independently of one another represent hydrogen, methyl, ethyl, n-propyl, isopropyl, tert-butyl, 2-fluoroethyl, cyclopropyl, cyclobutyl, cyclopropylmethyl, methoxy, ethoxy, methoxymethyl, monofluoromethyl, difluoromethyl, trifluoromethyl, difluoromethoxy, 3,3-difluorocyclobutylmethoxy, cyclobutylmethoxy, cyclopropylmethoxy, cyclopropylmethoxymethyl, cyclobutyloxymethyl, 3-fluorobutyloxymethyl, 3,3-difluorocyclobutylmethoxymethyl, 2,2,2-trifluoroethoxy, 2,2,2-trifluoroethoxymethyl, 2,2-difluorocyclopropylmethoxy, cyclobutyloxy, 3,3-difluorocyclobutyloxy, fluoromethylcyclopropylmethoxy, difluoromethylcyclopropylmethoxy, trifluoromethylcyclopropylmethoxy, fluorine;
n represents 0 or 1 and
m represents 1, and
muscarinic receptor antagonists selected from the group comprising Oxybutynin, R-Oxybutynin and Tolterodine,
and the salts, solvates and solvates of the salts thereof.

A further embodiment of the present invention relates to combinations of compounds of formula (I)

in which
X, Y and Z are selected such that the aromatic 5-membered ring has the structural formula h′)

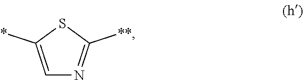

R₁ represents pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl, 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chiorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R₃ represents hydrogen;
R₅ represents hydrogen, fluorine;
R₆ represents a group of the formula a), c′) or c″)

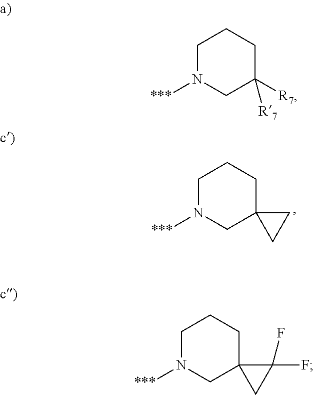

where *** marks the attachment to the adjacent piperidine ring,
where R₇ and R′₇ independently of one another represent hydrogen, methyl, ethyl, n-propyl, isopropyl, tert-butyl, 2-fluoroethyl, cyclopropyl, cyclobutyl, cyclopropylmethyl, methoxy, ethoxy, methoxymethyl, monofluoromethyl, difluoromethyl, trifluoromethyl, difluoromethoxy, 3,3-difluorocyclobutylmethoxy, cyclobutylmethoxy, cyclopropylmethoxy, cyclopropylmethoxymethyl, cyclobutyloxymethyl, 3-fluorobutyloxymethyl, 3,3-difluorocyclobutylmethoxymethyl, 2,2,2-trifluoroethoxy, 2,2,2-trifluoroethoxymethyl, 2,2-difluorocyclopropylmethoxy, cyclobutyloxy, 3,3-difluorocyclobutyloxy, fluoromethylcyclopropylmethoxy, difluoromethylcyclopropylmethoxy, trifluoromethylcyclopropylmethoxy, fluorine;
n represents 0 or 1 and
m represents 1, and
Oxybutynin,
and the salts, solvates and solvates of the salts thereof.

A further embodiment of the present invention relates to combinations of compounds of formula (I)

in which
X, Y and Z are selected such that the aromatic 5-membered ring has the structural formula h′)

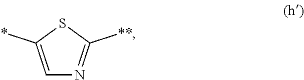

R₁ represents pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl, 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R₃ represents hydrogen;
R₅ represents hydrogen, fluorine;
R₆ represents a group of the formula a), c′) or c″)

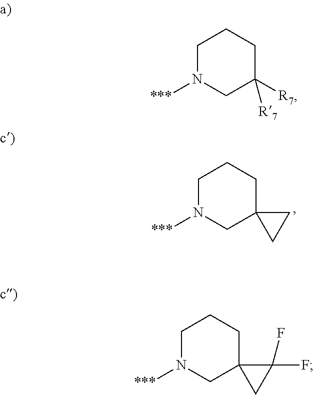

where *** marks the attachment to the adjacent piperidine ring,
where R₇ and R′₇ independently of one another represent hydrogen, methyl, ethyl, n-propyl, isopropyl, tert-butyl, 2-fluoroethyl, cyclopropyl, cyclobutyl, cyclopropylmethyl, methoxy, ethoxy, methoxymethyl, monofluoromethyl, difluoromethyl, trifluoromethyl, difluoromethoxy, 3,3-difluorocyclobutylmethoxy, cyclobutylmethoxy, cyclopropylmethoxy, cyclopropylmethoxymethyl, cyclobutyloxymethyl, 3-fluorobutyloxymethyl, 3,3-difluorocyclobutylmethoxymethyl, 2,2,2-trifluoroethoxy, 2,2,2-trifluoroethoxymethyl, 2,2-difluorocyclopropylmethoxy, cyclobutyloxy, 3,3-difluorocyclobutyloxy, fluoromethylcyclopropylmethoxy, difluoromethylcyclopropylmethoxy, trifluoromethylcyclopropylmethoxy, fluorine;
n represents 0 or 1 and
m represents 1, and
R-Oxybutynin,
and the salts, solvates and solvates of the salts thereof.

A further embodiment of the present invention relates to combinations of compounds of formula (I)

in which
X, Y and Z are selected such that the aromatic 5-membered ring has the structural formula h′)

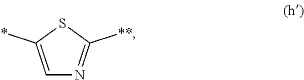

R₁ represents pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl, 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R₃ represents hydrogen;
R₅ represents hydrogen, fluorine;
R₆ represents a group of the formula a), c′) or c″)

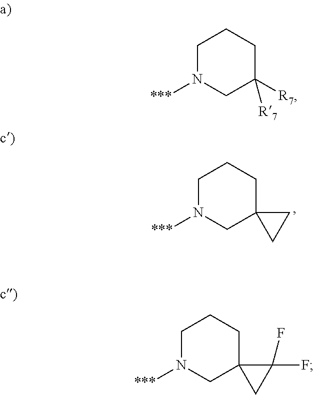

where *** marks the attachment to the adjacent piperidine ring,
where R₇ and R′₇ independently of one another represent hydrogen, methyl, ethyl, n-propyl, isopropyl, tert-butyl, 2-fluoroethyl, cyclopropyl, cyclobutyl, cyclopropylmethyl, methoxy, ethoxy, methoxymethyl, monofluoromethyl, difluoromethyl, trifluoromethyl, difluoromethoxy, 3,3-difluorocyclobutylmethoxy, cyclobutylmethoxy, cyclopropylmethoxy, cyclopropylmethoxymethyl, cyclobutyloxymethyl, 3-fluorobutyloxymethyl, 3,3-difluorocyclobutylmethoxymethyl, 2,2,2-trifluoroethoxy, 2,2,2-trifluoroethoxymethyl, 2,2-difluorocyclopropylmethoxy, cyclobutyloxy, 3,3-difluorocyclobutyloxy, fluoromethylcyclopropylmethoxy, difluoromethylcyclopropylmethoxy, trifluoromethylcyclopropylmethoxy, fluorine;
n represents 0 or 1 and
m represents 1, and
Tolterodine,
and the salts, solvates and solvates of the salts thereof.

A preferred embodiment of the present invention is directed to combinations of N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-[4-(3,4-dihydroisoquinolin-2(1H)-yl)piperidin-1-yl]-1,3-thiazole-5-carboxamide, 2-[3-(cyclopropylmethyl)[1,4′-bipiperidin]-1′-yl]-N-[(3,5-difluoropyridin-2-yl)methyl]-1,3-thiazole-5-carboxamide, 2-[3-(difluoromethyl)[1,4′-bipiperidin]-1′-yl]-N-[(3,5-difluoropyridin-2-yl)methyl]-1,3-thiazolo-5-carboxamide, N-[(3,5-difluoropyridin-2-yl)methyl]-2-[3-(trifluoromethyl)[1,4′-bipiperidin]-1′-yl]-1,3-thiazole-5-carboxamide, N-[(3,5-difluoropyridin-2-yl)methyl]-2-[3-(fluoromethyl)-[1,4′-bipiperidin]-1′-yl]-1,3-thiazole-5-carboxamide, 2-{3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-4-methyl-2-[(3R)-3-methyl[1,4′-bipiperidin]-1′-yl]-1,3-thiazole-5-carboxamide, N-[(3,5-difluoro-pyridin-2-yl)methyl]-4-methyl-2-[(3R)-3-methyl[1,4′-bipiperidin]-1′-yl]-1,3-thiazole-5-carboxamide, N-[(3,5-difluoropyridin-2-yl)methyl]-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-4-(trifluoromethyl)-1,3-thiazole-5-carboxamide, N-[(3,5-difluoropyridin-2-yl)methyl]-5-ethyl-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-1,3-oxazole-4-carboxamide, N-[(3,5-difluoropyridin-2-yl)methyl]-5-methyl-2-[(3R)-3-methyl[1,4′-bipiperidin]-1′-yl]-1,3-oxazole-4-carboxamide, N-[(3,5-difluoropyridin-2-yl)methyl]-2-[(3R)-3-methoxy[1,4′-bipiperidin]-1′-yl]-1,3-thiazole-5-carboxamide, 2-[3-(difluoromethoxy)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-(3-ethyl[1,4′-bipiperidin]-1′-yl)-1,3-thiazole-5-carboxamide, 2-[(3R)-3-methyl[1,4′-bipiperidin]-1′-yl]-N-{[4-(trifluoromethyl)pyridin-2-yl]methyl}-1,3-thiazole-5-carboxamide, 2-[(3R)-3-methyl[1,4′-bipiperidin]-1′-yl]-N-[3-(trifluoromethyl)benzyl]-1,3-thiazole-5-carboxamide, N-[(3-fluoropyridin-2-yl)methyl]-2-[(3R)-3-methyl[1,4′-bipiperidin]-1′-yl]-1,3-thiazole-5-carboxamide, N-(5-chloro-2-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[4-(trifluoromethyl)benzyl]-1,3-thiazole-5-carboxamide, N-[(5-chloro-3-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(3-methylpyridin-2-yl)methyl]-1,3-thiazole-5-carboxamide, 2-[(3R)-3-methyl[1,4′-bipiperidin]-1′-yl]-N-[(4-methylpyridin-2-yl)methyl]-1,3-thiazole-5-carboxamide, N-[(3-chloropyridin-2-yl)methyl]-2-[(3R)-3-methyl[1,4′-bipiperidin]-1′-yl]-1,3-thiazole-5-carboxamide, N-[(3-fluoropyridin-2-yl)methyl]-N-methyl-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethyl)pyridin-2-yl]methyl}-1,3-thiazole-5-carboxamide, N-[(5-chloropyridin-2-yl)methyl]-2-[(3R)-3-methyl[1,4′-bipiperidin]-1′-yl]-1,3-thiazole-5-carboxamide, N-[1-(2,5-difluorophenyl)ethyl]-2-[(3R)-3-methyl[1,4′-bipiperidin]-1′-yl]-1,3-thiazole-5-carboxamide, N-[(3-chloro-5-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethoxy)pyridin-2-yl]methyl}-1,3-thiazole-5-carboxamide, N-(4-chlorobenzyl)-2-[(3R)-3-methyl[1,4′-bipiperidin]-1′-yl]-1,3-thiazole-5-carboxamide, N-(2-chloro-5-fluorobenzyl)-2-[(3R)-3-methyl[1,4′-bipiperidin]-1′-yl]-1,3-thiazole-5-carboxamide, N-(4-methylbenzyl)-2-[(3R)-3-methyl[1,4′-bipiperidin]-1′-yl]-1,3-thiazole-5-carboxamide, N-(3-methylbenzyl)-2-[(3R)-3-methyl[1,4′-bipiperidin]-1′-yl]-1,3-thiazole-5-carboxamide, N-(2-methylbenzyl)-2-[(3R)-3-methyl[1,4′-bipiperidin]-1′-yl]-1,3-thiazole-5-carboxamide, 2-[(3S)-(difluoromethyl)[1,4′-bipiperidin]-1′-yl]-N-[(3,5-difluoropyridin-2-yl)methyl]-1,3-thiazole-5-carboxamide, 2-[(3R)-3-(difluoromethyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[(3S)-3-(fluoromethyl)[1,4′-bipiperidin]-1′-yl]-1,3-thiazole-5-carboxamide, N-[(3,5-difluoropyridin-2-yl)methyl]-2-[(3R)-3-(fluoromethyl)[1,4′-bipiperidin]-1′-yl]-1,3-thiazole-5-carboxamide, N-[(3,5-difluoropyridin-2-yl)methyl]-2-[(3S)-3-(trifluoromethyl)[1,4′-bipiperidin]-1′-yl]-1,3-thiazole-5-carboxamide, N-[(3,5-difluoropyridin-2-yl)methyl]-2-[(3R)-3-(trifluoromethyl)[1,4′-bipiperidin]-1′-yl]-1,3-thiazole-5-carboxamide, 2-{(3S)-3-[(3,3-difluorocyclobutyl)methoxy][1,4′-bipiperidin]-1′-yl}-N-[(3,5-difluoropyridin-2-yl)methyl]-1,3-thiazole-5-carboxamide, 2-{(3R)-3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(1S)-1-(2,5-difluorophenyl)ethyl]-2-[(3R)-3-methyl[1,4′-bipiperidin]-1′-yl]-1,3-thiazole-5-carboxamide, N-[(1R)-1-(2,5-difluorophenyl)ethyl]-2-[(3R)-3-methyl[1,4′-bipiperidin]-1′-yl]-1,3-thiazole-5-carboxamide, N-[(3,5-difluoropyridin-2-yl)methyl]-2-[3-(methoxymethyl)[1,4′-bipiperidin]-1′-yl]-1,3-thiazole-5-carboxamide, N-[(3,5-difluoropyridin-2-yl)methyl]-3-[(3R)-3-methyl[1,4′-bipiperidin]-1′-yl]-1,2,4-oxadiazole-5-carboxamide, N-[(3,5-difluoropyridin-2-yl)methyl]-2-[(3R)-3′-fluor-3-methyl[1,4′-bipiperidin]-1′-yl]-1,3-thiazole-5-carboxamide, ent-N-[(3,5-difluoropyridin-2-yl)methyl]-2-[(3R), (3′R)-3′-fluoro-3-methyl[1,4′-bipiperidin]-1′-yl]-1,3-thiazole-5-carboxamide, ent-N-[(3,5-difluoropyridin-2-yl)methyl]-2-[(3R), (3'S)-3′-fluoro-3-methyl[1,4′-bipiperidin]-1′-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[(5-fluoro-2-thienyl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-(pyridin-4-ylmethyl)-1,3-thiazole-5-carboxamide, N-[(3,5-difluoropyridin-2-yl)methyl]-2-{3-[(2,2,2-trifluoroethoxy)methyl][1,4′-bipiperidin]-1′-yl}-1,3-thiazole-5-carboxamide, N-[(3,5-difluoropyridin-2-yl)methyl]-2-[3-({[1-(fluoromethyl)cyclopropyl]methoxy}methyl)[1,4′-bipiperidin]-1′-yl]-1,3-thiazole-5-carboxamide, 2-[3-({[1-(difluoromethyl)cyclopropyl]methoxy}methyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[3-({[1-(trifluoromethyl)cyclopropyl]methoxy}methyl)[1,4′-bipiperidin]-1′-yl]-1,3-thiazole-5-carboxamide, N-[(3,5-difluoropyridin-2-yl)methyl]-2-(3,3-dimethyl[1,4′-bipiperidin]-1′-yl)-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, 2-[4-(1,1-difluoro-5-azaspiro[2.5]octan-5-yl)piperidin-1-yl]-N-[(3,5-difluoropyridin-2-yl)methyl]-1,3-thiazole-5-carboxamide, 2-[3-(cyclobutylmethoxy)[1,4′-bipiperidin]-1′-yl]-N-[(3,5-difluoropyridin-2-yl)methyl]-1,3-thiazole-5-carboxamide, 2-[3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclobutyloxy)methyl]-[1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[3-ethoxy[1,4′-bipiperidin]-1′-yl]-1,3-thiazole-5-carboxamide, N-[(3,5-difluoropyridin-2-yl)methyl]-2-{4-[(3R)-3-methylpiperidin-1-yl]azepan-1-yl}-1,3-thiazole-5-carboxamide, 2-[(3R)-3-methyl[1,4′-bipiperidin]-1′-yl]-N-[(6-methylpyridin-3-yl)methyl]-1,3-thiazole-5-carboxamide, N-benzyl-2-[(3R)-3-methyl[1,4′-bipiperidin]-1′-yl]-1,3-thiazole-5-carboxamide, N-[(3,5-difluoropyridin-2-yl)methyl]-2-[3-({[3-fluorobutyl]oxy}methyl)[1,4′-bipiperidin]-1′-yl]-1,3-thiazole-5-carboxamide, 2-(3-{[(3,3-difluorocyclobutyl)methoxy]methyl}[1,4′-bipiperidin]-1′-yl)-N-[(3,5-difluoropyridin-2-yl)methyl]-1,3-thiazole-5-carboxamide, N-[(3-fluoropyridin-4-yl)methyl]-2-[(3R)-3-methyl[1,4′-bipiperidin]-1′-yl]-1,3-thiazole-5-carboxamide, N-[(3,5-difluoropyridin-2-yl)methyl]-2-[3-(2,2,2-trifluoroethoxy)[1,4′-bipiperidin]-1′-yl]-1,3-thiazole-5-carboxamide, N-[(4,6-dimethylpyridin-3-yl)methyl]-2-[(3R)-3-methyl[1,4′-bipiperidin]-1′-yl]-1,3-thiazole-5-carboxamide, N-[(4-chloro-1-methyl-1H-pyrazol-5-yl)methyl]-2-[(3R)-3-methyl[1,4′-bipiperidin]-1′-yl]-1,3-thiazole-5-carboxamide, N-(3-methoxybenzyl)-2-[(3R)-3-methyl[1,4′-bipiperidin]-1′-yl]-1,3-thiazole-5-carboxamide, N-(2,5-difluorobenzyl)-2-[(3R)-3-methyl[1,4′-bipiperidin]-1′-yl]-1,3-thiazole-5-carboxamide, N-(3-hydroxybenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(2R)-2-phenylpropyl]-1,3-thiazole-5-carboxamide, N-(4-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-(pyridin-3-ylmethyl)-1,3-thiazole-5-carboxamide, N-(3-fluorobenzyl)-2-[(3R)-3-methyl[1,4′-bipiperidin]-1′-yl]-1,3-thiazole-5-carboxamide, N-(2-fluorobenzyl)-2-[(3R)-3-methyl[1,4′-bipiperidin]-1′-yl]-1,3-thiazole-5-carboxamide, N-(2-chloro-4-fluorophenyl)-2-[(3R)-3-methyl[1,4′-bipiperidin]-1′-yl]-1,3-thiazole-5-carboxamide, N-(3-cyano-4-fluorophenyl)-2-[(3R)-3-methyl[1,4′-bipiperidin]-1′-yl]-1,3-thiazole-5-carboxamide, N-methyl-2-[(3R)-3-methyl[1,4′-bipiperidin]-1′-yl]-N-(pyridin-3-ylmethyl)-1,3-thiazole-5-carboxamide, N-methyl-2-[(3R)-3-methyl[1,4′-bipiperidin]-1′-yl]-N-(pyridin-4-ylmethyl)-1,3-thiazole-5-carboxamide, N-benzyl-N-methyl-2-[(3R)-3-methyl[1,4′-bipiperidin]-1′-yl]-1,3-thiazole-5-carboxamide, N-(2-cyclopropylphenyl)-2-[(3R)-3-methyl[1,4′-bipiperidin]-1′-yl]-1,3-thiazole-5-carboxamide, N-(3-chlorobenzyl)-2-[(3R)-3-methyl[1,4′-bipiperidin]-1′-yl]-1,3-thiazol-5-carboxamide, 2-[(3R)-3-methyl[1,4′-bipiperidin]-1′-yl]-N-[(1R)-1-(4-methylphenyl)ethyl]-1,3-thiazole-5-carboxamide, N-(2-ethylpyridin-4-yl)-2-[(3R)-3-methyl[1,4′-bipiperidin]-1′-yl]-1,3-thiazole-5-carboxamide, N-[(3,5-difluoropyridin-2-yl)methyl]-2-[(3S)-3-(methoxymethyl)[1,4′-bipiperidin]-1′-yl]-1,3-thiazole-5-carboxamide, N-[(3,5-difluoropyridin-2-yl)methyl]-2-[(3R)-3-(methoxymethyl) [1,4′-bipiperidin]-1′-yl]-1,3-thiazole-5-carboxamide, 2-{(3S)-3-[(cyclobutyloxy)methyl][1,4′-bipiperidin]-1′-yl}-N-[(3,5-difluoropyridin-2-yl)methyl]-1,3-thiazole-5-carboxamide, 2-{(3R)-3-[(cyclobutyloxy)methyl][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-(3-isopropyl[1,4′-bipiperidin]-1′-yl)-1,3-thiazole-5-carboxamide, N-[(3,5-difluoropyridin-2-yl)methyl]-2-[4-((4S)-4-methylazepan-1-yl)piperidin-1-yl]-1,3-thiazole-5-carboxamide, N-[(3,5-difluoropyridin-2-yl)methyl]-2-[4-((4R)-4-methylazepan-1-yl)piperidin-1-yl]-1,3-thiazole-5-carboxamide, N-[(3,5-difluoropyridin-2-yl)methyl]-2-{(3S)-3-[(2,2,2-trifluoroethoxy)methyl][1,4′-bipiperidin]-1′-yl}-1,3-thiazole-5-carboxamide, N-[(3,5-difluoropyridin-2-yl)methyl]-2-{(3R)-3-[(2,2,2-trifluoroethoxy)methyl][1,4′-bipiperidin]-1′-yl}-1,3-thiazole-5-carboxamide, 2-{3-[(2,2-difluorocyclopropyl)methoxy][1,4′-bipiperidin]-1′-yl}-N-[(3,5-difluoropyridin-2-yl)methyl]-1,3-thiazole-5-carboxamide, 2-[3-(cyclobutyloxy)[1,4′-bipiperidin]-1′-yl]-N-[(3,5-difluoropyridin-2-yl)methyl]-1,3-thiazole-5-carboxamide, 2-{3-[(3,3-difluorocyclobutyl)oxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[(3R)-3′-fluoro-3-methyl[1,4′-bipiperidin]-1′-yl]-1,3-thiazole-4-carboxamide, N-[(3,5-difluoropyridin-2-yl)methyl]-2-[(3R)-3′-fluoro-3-methyl[1,4′-bipiperidin]-1′-yl]-1,3-oxazole-4-carboxamide, N-(5-chloro-2-fluorobenzyl)-2-[(3R)-3′-fluoro-3-methyl[1,4′-bipiperidin]-1′-yl]-1,3-thiazole-5-carboxamide, 2-[(3R)-3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[1-(2,5-difluorophenyl)ethyl]-2-[(3R)-3′-fluoro-3-methyl[1,4′-bipiperidin]-1′-yl]-1,3-thiazole-5-carboxamide, 4-(2-chlorophenyl)-N-[(3,5-difluoropyridin-2-yl)methyl]-2-[(3R)-3-methyl[1,4′-bipiperidin]-1′-yl]-1,3-thiazole-5-carboxamide, 4-bromo-N-[(3,5-difluoropyridin-2-yl)methyl]-2-[(3R)-3-methyl[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-(3-propyl[1,4′-bipiperidin]-1′-yl)-1,3-thiazole-5-carboxamide, 4-cyclopropyl-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-((3S)-3-ethoxy[1,4′-bipiperidin]-1′-yl)-1,3-thiazole-5-carboxamide, N-[(3,5-difluoropyridin-2-yl)methyl]-2-((3R)-3-ethoxy[1,4′-bipiperidin]-1′-yl)-1,3-thiazole-5-carboxamide, 2-[(3S)-3-(cyclobutylmethoxy)[1,4′-bipiperidin]-1′-yl]-N-[(3,5-difluoropyridin-2-yl)methyl]-1,3-thiazole-5-carboxamide, 2-[(3R)-3-(cyclobutylmethoxy)[1,4′-bipiperidin]-1′-yl]-N-[(3,5-difluoropyridin-2-yl)methyl]-1,3-thiazole-5-carboxamide, formic acid-N-[(3,5-difluoropyridin-2-yl)methyl]-2-[3-(2-fluoroethyl)[1,4′-bipiperidin]-1′-yl]-1,3-thiazole-5-carboxamide, 2-([1,4′-bipiperidin]-1′-yl)-N-[(3,5-difluoropyridin-2-yl)methyl]-1,3-thiazole-5-carboxamide, N-[1-(3,5-difluoropyridin-2-yl)cyclopropyl]-2-[(3R)-3-methyl[1,4′-bipiperidin]-1′-yl]-1,3-thiazole-5-carboxamide, N-[(3,5-difluoropyridin-2-yl)methyl]-4-ethyl-2-[(3R)-3-methyl[1,4′-bipiperidin]-1′-yl]-1,3-thiazole-5-carboxamide, 2-[4-(3S)-(1,1-difluoro-5-azaspiro[2.5]octan-5-yl)piperidin-1-yl]-N-[(3,5-difluoropyridin-2-yl)methyl]-1,3-thiazole-5-carboxamide, 2-[4-(3R)-(1,1-difluoro-5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-(3-phenyl[1,4′-bipiperidin]-1′-yl)-1,3-thiazole-5-carboxamide, 2-[4-(1,1-difluoro-5-azaspiro[2.5]octan-5-yl)-3-fluoropiperidin-1-yl]-N-[(3,5-difluoropyridin-2-yl)methyl]-1,3-thiazole-5-carboxamide, 2-[4-(5-azaspiro[2.5]octan-5-yl)-3-fluoropiperidin-1-yl]-N-[(3,5-difluoropyridin-2-yl)methyl]-1,3-thiazole-5-carboxamide,
as compound of formula (I),
and muscarinic receptor antagonists,
and the salts, solvates and solvates of the salts thereof.

A preferred embodiment of the present invention is directed to combinations of N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-[4-(3,4-dihydroisoquinolin-2(1H)-yl)piperidin-1-yl]-1,3-thiazole-5-carboxamide, 2-[3-(cyclopropylmethyl)[1,4′-bipiperidin]-1′-yl]-N-[(3,5-difluoropyridin-2-yl)methyl]-1,3-thiazole-5-carboxamide, 2-[3-(difluoromethyl)[1,4′-bipiperidin]-1′-yl]-N-[(3,5-difluoropyridin-2-yl)methyl]-1,3-thiazolo-5-carboxamide, N-[(3,5-difluoropyridin-2-yl)methyl]-2-[3-(trifluoromethyl)[1,4′-bipiperidin]-1′-yl]-1,3-thiazole-5-carboxamide, N-[(3,5-difluoropyridin-2-yl)methyl]-2-[3-(fluoromethyl)-[1,4′-bipiperidin]-1′-yl]-1,3-thiazole-5-carboxamide, 2-{3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-4-methyl-2-[(3R)-3-methyl[1,4′-bipiperidin]-1′-yl]-1,3-thiazole-5-carboxamide, N-[(3,5-difluoro-pyridin-2-yl)methyl]-4-methyl-2-[(3R)-3-methyl[1,4′-bipiperidin]-1′-yl]-1,3-thiazole-5-carboxamide, N-[(3,5-difluoropyridin-2-yl)methyl]-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-4-(trifluoromethyl)-1,3-thiazole-5-carboxamide, N-[(3,5-difluoropyridin-2-yl)methyl]-5-ethyl-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-1,3-oxazole-4-carboxamide, N-[(3,5-difluoropyridin-2-yl)methyl]-5-methyl-2-[(3R)-3-methyl[1,4′-bipiperidin]-1′-yl]-1,3-oxazole-4-carboxamide, N-[(3,5-difluoropyridin-2-yl)methyl]-2-[(3R)-3-methoxy[1,4′-bipiperidin]-1′-yl]-1,3-thiazole-5-carboxamide, 2-[3-(difluoromethoxy)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-(3-ethyl[1,4′-bipiperidin]-1′-yl)-1,3-thiazole-5-carboxamide, 2-[(3R)-3-methyl[1,4′-bipiperidin]-1′-yl]-N-{[4-(trifluoromethyl)pyridin-2-yl]methyl}-1,3-thiazole-5-carboxamide, 2-[(3R)-3-methyl[1,4′-bipiperidin]-1′-yl]-N-[3-(trifluoromethyl)benzyl]-1,3-thiazole-5-carboxamide, N-[(3-fluoropyridin-2-yl)methyl]-2-[(3R)-3-methyl[1,4′-bipiperidin]-1′-yl]-1,3-thiazole-5-carboxamide, N-(5-chloro-2-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[4-(trifluoromethyl)benzyl]-1,3-thiazole-5-carboxamide, N-[(5-chloro-3-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(3-methylpyridin-2-yl)methyl]-1,3-thiazole-5-carboxamide, 2-[(3R)-3-methyl[1,4′-bipiperidin]-1′-yl]-N-[(4-methylpyridin-2-yl)methyl]-1,3-thiazole-5-carboxamide, N-[(3-chloropyridin-2-yl)methyl]-2-[(3R)-3-methyl[1,4′-bipiperidin]-1′-yl]-1,3-thiazole-5-carboxamide, N-[(3-fluoropyridin-2-yl)methyl]-N-methyl-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethyl)pyridin-2-yl]methyl}-1,3-thiazole-5-carboxamide, N-[(5-chloropyridin-2-yl)methyl]-2-[(3R)-3-methyl[1,4′-bipiperidin]-1′-yl]-1,3-thiazole-5-carboxamide, N-[1-(2,5-difluorophenyl)ethyl]-2-[(3R)-3-methyl[1,4′-bipiperidin]-1′-yl]-1,3-thiazole-5-carboxamide, N-[(3-chloro-5-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethoxy)pyridin-2-yl]methyl}-1,3-thiazole-5-carboxamide, N-(4-chlorobenzyl)-2-[(3R)-3-methyl[1,4′-bipiperidin]-1′-yl]-1,3-thiazole-5-carboxamide, N-(2-chloro-5-fluorobenzyl)-2-[(3R)-3-methyl[1,4′-bipiperidin]-1′-yl]-1,3-thiazole-5-carboxamide, N-(4-methylbenzyl)-2-[(3R)-3-methyl[1,4′-bipiperidin]-1′-yl]-1,3-thiazole-5-carboxamide, N-(3-methylbenzyl)-2-[(3R)-3-methyl[1,4′-bipiperidin]-1′-yl]-1,3-thiazole-5-carboxamide, N-(2-methylbenzyl)-2-[(3R)-3-methyl[1,4′-bipiperidin]-1′-yl]-1,3-thiazole-5-carboxamide, 2-[(3S)-(difluoromethyl)[1,4′-bipiperidin]-1′-yl]-N-[(3,5-difluoropyridin-2-yl)methyl]-1,3-thiazole-5-carboxamide, 2-[(3R)-3-(difluoromethyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[(3S)-3-(fluoromethyl)[1,4′-bipiperidin]-1′-yl]-1,3-thiazole-5-carboxamide, N-[(3,5-difluoropyridin-2-yl)methyl]-2-[(3R)-3-(fluoromethyl)[1,4′-bipiperidin]-1′-yl]-1,3-thiazole-5-carboxamide, N-[(3,5-difluoropyridin-2-yl)methyl]-2-[(3S)-3-(trifluoromethyl)[1,4′-bipiperidin]-1′-yl]-1,3-thiazole-5-carboxamide, N-[(3,5-difluoropyridin-2-yl)methyl]-2-[(3R)-3-(trifluoromethyl)[1,4′-bipiperidin]-1′-yl]-1,3-thiazole-5-carboxamide, 2-{(3S)-3-[(3,3-difluorocyclobutyl)methoxy][1,4′-bipiperidin]-1′-yl}-N-[(3,5-difluoropyridin-2-yl)methyl]-1,3-thiazole-5-carboxamide, 2-{(3R)-3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(1S)-1-(2,5-difluorophenyl)ethyl]-2-[(3R)-3-methyl[1,4′-bipiperidin]-1′-yl]-1,3-thiazole-5-carboxamide, N-[(1R)-1-(2,5-difluorophenyl)ethyl]-2-[(3R)-3-methyl[1,4′-bipiperidin]-1′-yl]-1,3-thiazole-5-carboxamide, N-[(3,5-difluoropyridin-2-yl)methyl]-2-[3-(methoxymethyl)[1,4′-bipiperidin]-1′-yl]-1,3-thiazole-5-carboxamide, N-[(3,5-difluoropyridin-2-yl)methyl]-3-[(3R)-3-methyl[1,4′-bipiperidin]-1′-yl]-1,2,4-oxadiazole-5-carboxamide, N-[(3,5-difluoropyridin-2-yl)methyl]-2-[(3R)-3′-fluor-3-methyl[1,4′-bipiperidin]-1′-yl]-1,3-thiazole-5-carboxamide, ent-N-[(3,5-difluoropyridin-2-yl)methyl]-2-[(3R), (3′R)-3′-fluoro-3-methyl[1,4′-bipiperidin]-1′-yl]-1,3-thiazole-5-carboxamide, ent-N-[(3,5-difluoropyridin-2-yl)methyl]-2-[(3R), (3'S)-3′-fluoro-3-methyl[1,4′-bipiperidin]-1′-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[(5-fluoro-2-thienyl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-(pyridin-4-ylmethyl)-1,3-thiazole-5-carboxamide, N-[(3,5-difluoropyridin-2-yl)methyl]-2-{3-[(2,2,2-trifluoroethoxy)methyl][1,4′-bipiperidin]-1′-yl}-1,3-thiazole-5-carboxamide, N-[(3,5-difluoropyridin-2-yl)methyl]-2-[3-({[1-(fluoromethyl)cyclopropyl]methoxy}methyl)[1,4′-bipiperidin]-1′-yl]-1,3-thiazole-5-carboxamide, 2-[3-({[1-(difluoromethyl)cyclopropyl]methoxy}methyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[3-({[1-(trifluoromethyl)cyclopropyl]methoxy}methyl)[1,4′-bipiperidin]-1′-yl]-1,3-thiazole-5-carboxamide, N-[(3,5-difluoropyridin-2-yl)methyl]-2-(3,3-dimethyl[1,4′-bipiperidin]-1′-yl)-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, 2-[4-(1,1-difluoro-5-azaspiro[2.5]octan-5-yl)piperidin-1-yl]-N-[(3,5-difluoropyridin-2-yl)methyl]-1,3-thiazole-5-carboxamide, 2-[3-(cyclobutylmethoxy)[1,4′-bipiperidin]-1′-yl]-N-[(3,5-difluoropyridin-2-yl)methyl]-1,3-thiazole-5-carboxamide, 2-[3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclobutyloxy)methyl]-[1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[3-ethoxy[1,4′-bipiperidin]-1′-yl]-1,3-thiazole-5-carboxamide, N-[(3,5-difluoropyridin-2-yl)methyl]-2-{4-[(3R)-3-methylpiperidin-1-yl]azepan-1-yl}-1,3-thiazole-5-carboxamide, 2-[(3R)-3-methyl[1,4′-bipiperidin]-1′-yl]-N-[(6-methylpyridin-3-yl)methyl]-1,3-thiazole-5-carboxamide, N-benzyl-2-[(3R)-3-methyl[1,4′-bipiperidin]-1′-yl]-1,3-thiazole-5-carboxamide, N-[(3,5-difluoropyridin-2-yl)methyl]-2-[3-({[3-fluorobutyl]oxy}methyl)[1,4′-bipiperidin]-1′-yl]-1,3-thiazole-5-carboxamide, 2-(3-{[(3,3-difluorocyclobutyl)methoxy]methyl}[1,4′-bipiperidin]-1′-yl)-N-[(3,5-difluoropyridin-2-yl)methyl]-1,3-thiazole-5-carboxamide, N-[(3-fluoropyridin-4-yl)methyl]-2-[(3R)-3-methyl[1,4′-bipiperidin]-1′-yl]-1,3-thiazole-5-carboxamide, N-[(3,5-difluoropyridin-2-yl)methyl]-2-[3-(2,2,2-trifluoroethoxy)[1,4′-bipiperidin]-1′-yl]-1,3-thiazole-5-carboxamide, N-[(4,6-dimethylpyridin-3-yl)methyl]-2-[(3R)-3-methyl[1,4′-bipiperidin]-1′-yl]-1,3-thiazole-5-carboxamide, N-[(4-chloro-1-methyl-1H-pyrazol-5-yl)methyl]-2-[(3R)-3-methyl[1,4′-bipiperidin]-1′-yl]-1,3-thiazole-5-carboxamide, N-(3-methoxybenzyl)-2-[(3R)-3-methyl[1,4′-bipiperidin]-1′-yl]-1,3-thiazole-5-carboxamide, N-(2,5-difluorobenzyl)-2-[(3R)-3-methyl[1,4′-bipiperidin]-1′-yl]-1,3-thiazole-5-carboxamide, N-(3-hydroxybenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(2R)-2-phenylpropyl]-1,3-thiazole-5-carboxamide, N-(4-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-(pyridin-3-ylmethyl)-1,3-thiazole-5-carboxamide, N-(3-fluorobenzyl)-2-[(3R)-3-methyl[1,4′-bipiperidin]-1′-yl]-1,3-thiazole-5-carboxamide, N-(2-fluorobenzyl)-2-[(3R)-3-methyl[1,4′-bipiperidin]-1′-yl]-1,3-thiazole-5-carboxamide, N-(2-chloro-4-fluorophenyl)-2-[(3R)-3-methyl[1,4′-bipiperidin]-1′-yl]-1,3-thiazole-5-carboxamide, N-(3-cyano-4-fluorophenyl)-2-[(3R)-3-methyl[1,4′-bipiperidin]-1′-yl]-1,3-thiazole-5-carboxamide, N-methyl-2-[(3R)-3-methyl[1,4′-bipiperidin]-1′-yl]-N-(pyridin-3-ylmethyl)-1,3-thiazole-5-carboxamide, N-methyl-2-[(3R)-3-methyl[1,4′-bipiperidin]-1′-yl]-N-(pyridin-4-ylmethyl)-1,3-thiazole-5-carboxamide, N-benzyl-N-methyl-2-[(3R)-3-methyl[1,4′-bipiperidin]-1′-yl]-1,3-thiazole-5-carboxamide, N-(2-cyclopropylphenyl)-2-[(3R)-3-methyl[1,4′-bipiperidin]-1′-yl]-1,3-thiazole-5-carboxamide, N-(3-chlorobenzyl)-2-[(3R)-3-methyl[1,4′-bipiperidin]-1′-yl]-1,3-thiazol-5-carboxamide, 2-[(3R)-3-methyl[1,4′-bipiperidin]-1′-yl]-N-[(1R)-1-(4-methylphenyl)ethyl]-1,3-thiazole-5-carboxamide, N-(2-ethylpyridin-4-yl)-2-[(3R)-3-methyl[1,4′-bipiperidin]-1′-yl]-1,3-thiazole-5-carboxamide, N-[(3,5-difluoropyridin-2-yl)methyl]-2-[(3S)-3-(methoxymethyl)[1,4′-bipiperidin]-1′-yl]-1,3-thiazole-5-carboxamide, N-[(3,5-difluoropyridin-2-yl)methyl]-2-[(3R)-3-(methoxymethyl)[1,4′-bipiperidin]-1′-yl]-1,3-thiazole-5-carboxamide, 2-[(3S)-3-[(cyclobutyloxy)methyl][1,4′-bipiperidin]-1′-yl]-N-[(3,5-difluoropyridin-2-yl)methyl]-1,3-thiazole-5-carboxamide, 2-{(3R)-3-[(cyclobutyloxy)methyl][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-(3-isopropyl[1,4′-bipiperidin]-1′-yl)-1,3-thiazole-5-carboxamide, N-[(3,5-difluoropyridin-2-yl)methyl]-2-[4-((4S)-4-methylazepan-1-yl)piperidin-1-yl]-1,3-thiazole-5-carboxamide, N-[(3,5-difluoropyridin-2-yl)methyl]-2-[4-((4R)-4-methylazepan-1-yl)piperidin-1-yl]-1,3-thiazole-5-carboxamide, N-[(3,5-difluoropyridin-2-yl)methyl]-2-{(3S)-3-[(2,2,2-trifluoroethoxy)methyl][1,4′-bipiperidin]-1′-yl}-1,3-thiazole-5-carboxamide, N-[(3,5-difluoropyridin-2-yl)methyl]-2-{(3R)-3-[(2,2,2-trifluoroethoxy)methyl][1,4′-bipiperidin]-1′-yl}-1,3-thiazole-5-carboxamide, 2-{3-[(2,2-difluorocyclopropyl)methoxy][1,4′-bipiperidin]-1′-yl}-N-[(3,5-difluoropyridin-2-yl)methyl]-1,3-thiazole-5-carboxamide, 2-[3-(cyclobutyloxy)[1,4′-bipiperidin]-1′-yl]-N-[(3,5-difluoropyridin-2-yl)methyl]-1,3-thiazole-5-carboxamide, 2-{3-[(3,3-difluorocyclobutyl)oxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[(3R)-3′-fluoro-3-methyl[1,4′-bipiperidin]-1′-yl]-1,3-thiazole-4-carboxamide, N-[(3,5-difluoropyridin-2-yl)methyl]-2-[(3R)-3′-fluoro-3-methyl[1,4′-bipiperidin]-1′-yl]-1,3-oxazole-4-carboxamide, N-(5-chloro-2-fluorobenzyl)-2-[(3R)-3′-fluoro-3-methyl[1,4′-bipiperidin]-1′-yl]-1,3-thiazole-5-carboxamide, 2-[(3R)-3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[1-(2,5-difluorophenyl)ethyl]-2-[(3R)-3′-fluoro-3-methyl[1,4′-bipiperidin]-1′-yl]-1,3-thiazole-5-carboxamide, 4-(2-chlorophenyl)-N-[(3,5-difluoropyridin-2-yl)methyl]-2-[(3R)-3-methyl[1,4′-bipiperidin]-1′-yl]-1,3-thiazole-5-carboxamide, 4-bromo-N-[(3,5-difluoropyridin-2-yl)methyl]-2-[(3R)-3-methyl[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-(3-propyl[1,4′-bipiperidin]-1′-yl)-1,3-thiazole-5-carboxamide, 4-cyclopropyl-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-((3S)-3-ethoxy[1,4′-bipiperidin]-1′-yl)-1,3-thiazole-5-carboxamide, N-[(3,5-difluoropyridin-2-yl)methyl]-2-((3R)-3-ethoxy[1,4′-bipiperidin]-1′-yl)-1,3-thiazole-5-carboxamide, 2-[(3S)-3-(cyclobutylmethoxy)[1,4′-bipiperidin]-1′-yl]-N-[(3,5-difluoropyridin-2-yl)methyl]-1,3-thiazole-5-carboxamide, 2-[(3R)-3-(cyclobutylmethoxy)[1,4′-bipiperidin]-1′-yl]-N-[(3,5-difluoropyridin-2-yl)methyl]-1,3-thiazole-5-carboxamide, formic acid-N-[(3,5-difluoropyridin-2-yl)methyl]-2-[3-(2-fluoroethyl)[1,4′-bipiperidin]-1′-yl]-1,3-thiazole-5-carboxamide, 2-([1,4′-bipiperidin]-1′-yl)-N-[(3,5-difluoropyridin-2-yl)methyl]-1,3-thiazole-5-carboxamide, N-[1-(3,5-difluoropyridin-2-yl)cyclopropyl]-2-[(3R)-3-methyl[1,4′-bipiperidin]-1′-yl]-1,3-thiazole-5-carboxamide, N-[(3,5-difluoropyridin-2-yl)methyl]-4-ethyl-2-[(3R)-3-methyl[1,4′-bipiperidin]-1′-yl]-1,3-thiazole-5-carboxamide, 2-[4-(3S)-(1,1-difluoro-5-azaspiro[2.5]octan-5-yl)piperidin-1-yl]-N-[(3,5-difluoropyridin-2-yl)methyl]-1,3-thiazole-5-carboxamide, 2-[4-(3R)-(1,1-difluoro-5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-(3-phenyl[1,4′-bipiperidin]-1′-yl)-1,3-thiazole-5-carboxamide, 2-[4-(1,1-difluoro-5-azaspiro[2.5]octan-5-yl)-3-fluoropiperidin-1-yl]-N-[(3,5-difluoropyridin-2-yl)methyl]-1,3-thiazole-5-carboxamide, 2-[4-(5-azaspiro[2.5]octan-5-yl)-3-fluoropiperidin-1-yl]-N-[(3,5-difluoropyridin-2-yl)methyl]-1,3-thiazole-5-carboxamide
as compound of formula (I),
and muscarinic receptor antagonists selected from the group comprising Oxybutynin, R-Oxybutynin and Tolterodine,
and the salts, solvates and solvates of the salts thereof.

A preferred embodiment of the present invention is directed to combinations of N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-[4-(3,4-dihydroisoquinolin-2(1H)-yl)piperidin-1-yl]-1,3-thiazole-5-carboxamide, 2-[3-(cyclopropylmethyl)[1,4′-bipiperidin]-1′-yl]-N-[(3,5-difluoropyridin-2-yl)methyl]-1,3-thiazole-5-carboxamide, 2-[3-(difluoromethyl)[1,4′-bipiperidin]-1′-yl]-N-[(3,5-difluoropyridin-2-yl)methyl]-1,3-thiazolo-5-carboxamide, N-[(3,5-difluoropyridin-2-yl)methyl]-2-[3-(trifluoromethyl)[1,4′-bipiperidin]-1′-yl]-1,3-thiazole-5-carboxamide, N-[(3,5-difluoropyridin-2-yl)methyl]-2-[3-(fluoromethyl)-[1,4′-bipiperidin]-1′-yl]-1,3-thiazole-5-carboxamide, 2-{3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-4-methyl-2-[(3R)-3-methyl[1,4′-bipiperidin]-1′-yl]-1,3-thiazole-5-carboxamide, N-[(3,5-difluoro-pyridin-2-yl)methyl]-4-methyl-2-[(3R)-3-methyl[1,4′-bipiperidin]-1′-yl]-1,3-thiazole-5-carboxamide, N-[(3,5-difluoropyridin-2-yl)methyl]-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-4-(trifluoromethyl)-1,3-thiazole-5-carboxamide, N-[(3,5-difluoropyridin-2-yl)methyl]-5-ethyl-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-1,3-oxazole-4-carboxamide, N-[(3,5-difluoropyridin-2-yl)methyl]-5-methyl-2-[(3R)-3-methyl[1,4′-bipiperidin]-1′-yl]-1,3-oxazole-4-carboxamide, N-[(3,5-difluoropyridin-2-yl)methyl]-2-[(3R)-3-methoxy[1,4′-bipiperidin]-1′-yl]-1,3-thiazole-5-carboxamide, 2-[3-(difluoromethoxy)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-(3-ethyl[1,4′-bipiperidin]-1′-yl)-1,3-thiazole-5-carboxamide, 2-[(3R)-3-methyl[1,4′-bipiperidin]-1′-yl]-N-{[4-(trifluoromethyl)pyridin-2-yl]methyl}-1,3-thiazole-5-carboxamide, 2-[(3R)-3-methyl[1,4′-bipiperidin]-1′-yl]-N-[3-(trifluoromethyl)benzyl]-1,3-thiazole-5-carboxamide, N-[(3-fluoropyridin-2-yl)methyl]-2-[(3R)-3-methyl[1,4′-bipiperidin]-1′-yl]-1,3-thiazole-5-carboxamide, N-(5-chloro-2-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[4-(trifluoromethyl)benzyl]-1,3-thiazole-5-carboxamide, N-[(5-chloro-3-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(3-methylpyridin-2-yl)methyl]-1,3-thiazole-5-carboxamide, 2-[(3R)-3-methyl[1,4′-bipiperidin]-1′-yl]-N-[(4-methylpyridin-2-yl)methyl]-1,3-thiazole-5-carboxamide, N-[(3-chloropyridin-2-yl)methyl]-2-[(3R)-3-methyl[1,4′-bipiperidin]-1′-yl]-1,3-thiazole-5-carboxamide, N-[(3-fluoropyridin-2-yl)methyl]-N-methyl-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethyl)pyridin-2-yl]methyl}-1,3-thiazole-5-carboxamide, N-[(5-chloropyridin-2-yl)methyl]-2-[(3R)-3-methyl[1,4′-bipiperidin]-1′-yl]-1,3-thiazole-5-carboxamide, N-[1-(2,5-difluorophenyl)ethyl]-2-[(3R)-3-methyl[1,4′-bipiperidin]-1′-yl]-1,3-thiazole-5-carboxamide, N-[(3-chloro-5-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethoxy)pyridin-2-yl]methyl}-1,3-thiazole-5-carboxamide, N-(4-chlorobenzyl)-2-[(3R)-3-methyl[1,4′-bipiperidin]-1′-yl]-1,3-thiazole-5-carboxamide, N-(2-chloro-5-fluorobenzyl)-2-[(3R)-3-methyl[1,4′-bipiperidin]-1′-yl]-1,3-thiazole-5-carboxamide, N-(4-methylbenzyl)-2-[(3R)-3-methyl[1,4′-bipiperidin]-1′-yl]-1,3-thiazole-5-carboxamide, N-(3-methylbenzyl)-2-[(3R)-3-methyl[1,4′-bipiperidin]-1′-yl]-1,3-thiazole-5-carboxamide, N-(2-methylbenzyl)-2-[(3R)-3-methyl[1,4′-bipiperidin]-1′-yl]-1,3-thiazole-5-carboxamide, 2-[(3S)-(difluoromethyl)[1,4′-bipiperidin]-1′-yl]-N-[(3,5-difluoropyridin-2-yl)methyl]-1,3-thiazole-5-carboxamide, 2-[(3R)-3-(difluoromethyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[(3S)-3-(fluoromethyl)[1,4′-bipiperidin]-1′-yl]-1,3-thiazole-5-carboxamide, N-[(3,5-difluoropyridin-2-yl)methyl]-2-[(3R)-3-(fluoromethyl)[1,4′-bipiperidin]-1′-yl]-1,3-thiazole-5-carboxamide, N-[(3,5-difluoropyridin-2-yl)methyl]-2-[(3S)-3-(trifluoromethyl)[1,4′-bipiperidin]-1′-yl]-1,3-thiazole-5-carboxamide, N-[(3,5-difluoropyridin-2-yl)methyl]-2-[(3R)-3-(trifluoromethyl)[1,4′-bipiperidin]-1′-yl]-1,3-thiazole-5-carboxamide, 2-{(3S)-3-[(3,3-difluorocyclobutyl)methoxy][1,4′-bipiperidin]-1′-yl}-N-[(3,5-difluoropyridin-2-yl)methyl]-1,3-thiazole-5-carboxamide, 2-{(3R)-3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(1S)-1-(2,5-difluorophenyl)ethyl]-2-[(3R)-3-methyl[1,4′-bipiperidin]-1′-yl]-1,3-thiazole-5-carboxamide, N-[(1R)-1-(2,5-difluorophenyl)ethyl]-2-[(3R)-3-methyl[1,4′-bipiperidin]-1′-yl]-1,3-thiazole-5-carboxamide, N-[(3,5-difluoropyridin-2-yl)methyl]-2-[3-(methoxymethyl)[1,4′-bipiperidin]-1′-yl]-1,3-thiazole-5-carboxamide, N-[(3,5-difluoropyridin-2-yl)methyl]-3-[(3R)-3-methyl[1,4′-bipiperidin]-1′-yl]-1,2,4-oxadiazole-5-carboxamide, N-[(3,5-difluoropyridin-2-yl)methyl]-2-[(3R)-3′-fluor-3-methyl[1,4′-bipiperidin]-1′-yl]-1,3-thiazole-5-carboxamide, ent-N-[(3,5-difluoropyridin-2-yl)methyl]-2-[(3R), (3′R)-3′-fluoro-3-methyl[1,4′-bipiperidin]-1′-yl]-1,3-thiazole-5-carboxamide, ent-N-[(3,5-difluoropyridin-2-yl)methyl]-2-[(3R), (3'S)-3′-fluoro-3-methyl[1,4′-bipiperidin]-1′-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[(5-fluoro-2-thienyl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-(pyridin-4-ylmethyl)-1,3-thiazole-5-carboxamide, N-[(3,5-difluoropyridin-2-yl)methyl]-2-{3-[(2,2,2-trifluoroethoxy)methyl][1,4′-bipiperidin]-1′-yl}-1,3-thiazole-5-carboxamide, N-[(3,5-difluoropyridin-2-yl)methyl]-2-[3-({[1-(fluoromethyl)cyclopropyl]methoxy}methyl)[1,4′-bipiperidin]-1′-yl]-1,3-thiazole-5-carboxamide, 2-[3-({[1-(difluoromethyl)cyclopropyl]methoxy}methyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[3-({[1-(trifluoromethyl)cyclopropyl]methoxy}methyl)[1,4′-bipiperidin]-1′-yl]-1,3-thiazole-5-carboxamide, N-[(3,5-difluoropyridin-2-yl)methyl]-2-(3,3-dimethyl[1,4′-bipiperidin]-1′-yl)-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, 2-[4-(1,1-difluoro-5-azaspiro[2.5]octan-5-yl)piperidin-1-yl]-N-[(3,5-difluoropyridin-2-yl)methyl]-1,3-thiazole-5-carboxamide, 2-[3-(cyclobutylmethoxy)[1,4′-bipiperidin]-1′-yl]-N-[(3,5-difluoropyridin-2-yl)methyl]-1,3-thiazole-5-carboxamide, 2-[3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclobutyloxy)methyl]-[1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[3-ethoxy[1,4′-bipiperidin]-1′-yl]-1,3-thiazole-5-carboxamide, N-[(3,5-difluoropyridin-2-yl)methyl]-2-{4-[(3R)-3-methylpiperidin-1-yl]azepan-1-yl}-1,3-thiazole-5-carboxamide, 2-[(3R)-3-methyl[1,4′-bipiperidin]-1′-yl]-N-[(6-methylpyridin-3-yl)methyl]-1,3-thiazole-5-carboxamide, N-benzyl-2-[(3R)-3-methyl[1,4′-bipiperidin]-1′-yl]-1,3-thiazole-5-carboxamide, N-[(3,5-difluoropyridin-2-yl)methyl]-2-[3-({[3-fluorobutyl]oxy}methyl)[1,4′-bipiperidin]-1′-yl]-1,3-thiazole-5-carboxamide, 2-(3-{[(3,3-difluorocyclobutyl)methoxy]methyl}[1,4′-bipiperidin]-1′-yl)-N-[(3,5-difluoropyridin-2-yl)methyl]-1,3-thiazole-5-carboxamide, N-[(3-fluoropyridin-4-yl)methyl]-2-[(3R)-3-methyl[1,4′-bipiperidin]-1′-yl]-1,3-thiazole-5-carboxamide, N-[(3,5-difluoropyridin-2-yl)methyl]-2-[3-(2,2,2-trifluoroethoxy)[1,4′-bipiperidin]-1′-yl]-1,3-thiazole-5-carboxamide, N-[(4,6-dimethylpyridin-3-yl)methyl]-2-[(3R)-3-methyl[1,4′-bipiperidin]-1′-yl]-1,3-thiazole-5-carboxamide, N-[(4-chloro-1-methyl-1H-pyrazol-5-yl)methyl]-2-[(3R)-3-methyl[1,4′-bipiperidin]-1′-yl]-1,3-thiazole-5-carboxamide, N-(3-methoxybenzyl)-2-[(3R)-3-methyl[1,4′-bipiperidin]-1′-yl]-1,3-thiazole-5-carboxamide, N-(2,5-difluorobenzyl)-2-[(3R)-3-methyl[1,4′-bipiperidin]-1′-yl]-1,3-thiazole-5-carboxamide, N-(3-hydroxybenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(2R)-2-phenylpropyl]-1,3-thiazole-5-carboxamide, N-(4-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-(pyridin-3-ylmethyl)-1,3-thiazole-5-carboxamide, N-(3-fluorobenzyl)-2-[(3R)-3-methyl[1,4′-bipiperidin]-1′-yl]-1,3-thiazole-5-carboxamide, N-(2-fluorobenzyl)-2-[(3R)-3-methyl[1,4′-bipiperidin]-1′-yl]-1,3-thiazole-5-carboxamide, N-(2-chloro-4-fluorophenyl)-2-[(3R)-3-methyl[1,4′-bipiperidin]-1′-yl]-1,3-thiazole-5-carboxamide, N-(3-cyano-4-fluorophenyl)-2-[(3R)-3-methyl[1,4′-bipiperidin]-1′-yl]-1,3-thiazole-5-carboxamide, N-methyl-2-[(3R)-3-methyl[1,4′-bipiperidin]-1′-yl]-N-(pyridin-3-ylmethyl)-1,3-thiazole-5-carboxamide, N-methyl-2-[(3R)-3-methyl[1,4′-bipiperidin]-1′-yl]-N-(pyridin-4-ylmethyl)-1,3-thiazole-5-carboxamide, N-benzyl-N-methyl-2-[(3R)-3-methyl[1,4′-bipiperidin]-1′-yl]-1,3-thiazole-5-carboxamide, N-(2-cyclopropylphenyl)-2-[(3R)-3-methyl[1,4′-bipiperidin]-1′-yl]-1,3-thiazole-5-carboxamide, N-(3-chlorobenzyl)-2-[(3R)-3-methyl[1,4′-bipiperidin]-1′-yl]-1,3-thiazol-5-carboxamide, 2-[(3R)-3-methyl[1,4′-bipiperidin]-1′-yl]-N-[(1R)-1-(4-methylphenyl)ethyl]-1,3-thiazole-5-carboxamide, N-(2-ethylpyridin-4-yl)-2-[(3R)-3-methyl[1,4′-bipiperidin]-1′-yl]-1,3-thiazole-5-carboxamide, N-[(3,5-difluoropyridin-2-yl)methyl]-2-[(3S)-3-(methoxymethyl)[1,4′-bipiperidin]-1′-yl]-1,3-thiazole-5-carboxamide, N-[(3,5-difluoropyridin-2-yl)methyl]-2-[(3R)-3-(methoxymethyl)[1,4′-bipiperidin]-1′-yl]-1,3-thiazole-5-carboxamide, 2-{(3S)-3-[(cyclobutyloxy)methyl][1,4′-bipiperidin]-1′-yl}-N-[(3,5-difluoropyridin-2-yl)methyl]-1,3-thiazole-5-carboxamide, 2-{(3R)-3-[(cyclobutyloxy)methyl][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-(3-isopropyl[1,4′-bipiperidin]-1′-yl)-1,3-thiazole-5-carboxamide, N-[(3,5-difluoropyridin-2-yl)methyl]-2-[4-((4S)-4-methylazepan-1-yl)piperidin-1-yl]-1,3-thiazole-5-carboxamide, N-[(3,5-difluoropyridin-2-yl)methyl]-2-[4-((4R)-4-methylazepan-1-yl)piperidin-1-yl]-1,3-thiazole-5-carboxamide, N-[(3,5-difluoropyridin-2-yl)methyl]-2-{(3S)-3-[(2,2,2-trifluoroethoxy)methyl][1,4′-bipiperidin]-1′-yl}-1,3-thiazole-5-carboxamide, N-[(3,5-difluoropyridin-2-yl)methyl]-2-{(3R)-3-[(2,2,2-trifluoroethoxy)methyl][1,4′-bipiperidin]-1′-yl}-1,3-thiazole-5-carboxamide, 2-{3-[(2,2-difluorocyclopropyl)methoxy][1,4′-bipiperidin]-1′-yl}-N-[(3,5-difluoropyridin-2-yl)methyl]-1,3-thiazole-5-carboxamide, 2-[3-(cyclobutyloxy)[1,4′-bipiperidin]-1′-yl]-N-[(3,5-difluoropyridin-2-yl)methyl]-1,3-thiazole-5-carboxamide, 2-{3-[(3,3-difluorocyclobutyl)oxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[(3R)-3′-fluoro-3-methyl[1,4′-bipiperidin]-1′-yl]-1,3-thiazole-4-carboxamide, N-[(3,5-difluoropyridin-2-yl)methyl]-2-[(3R)-3′-fluoro-3-methyl[1,4′-bipiperidin]-1′-yl]-1,3-oxazole-4-carboxamide, N-(5-chloro-2-fluorobenzyl)-2-[(3R)-3′-fluoro-3-methyl[1,4′-bipiperidin]-1′-yl]-1,3-thiazole-5-carboxamide, 2-[(3R)-3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[1-(2,5-difluorophenyl)ethyl]-2-[(3R)-3′-fluoro-3-methyl[1,4′-bipiperidin]-1′-yl]-1,3-thiazole-5-carboxamide, 4-(2-chlorophenyl)-N-[(3,5-difluoropyridin-2-yl)methyl]-2-[(3R)-3-methyl[1,4′-bipiperidin]-1′-yl]-1,3-thiazole-5-carboxamide, 4-bromo-N-[(3,5-difluoropyridin-2-yl)methyl]-2-[(3R)-3-methyl[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-(3-propyl[1,4′-bipiperidin]-1′-yl)-1,3-thiazole-5-carboxamide, 4-cyclopropyl-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-((3S)-3-ethoxy[1,4′-bipiperidin]-1′-yl)-1,3-thiazole-5-carboxamide, N-[(3,5-difluoropyridin-2-yl)methyl]-2-((3R)-3-ethoxy[1,4′-bipiperidin]-1′-yl)-1,3-thiazole-5-carboxamide, 2-[(3S)-3-(cyclobutylmethoxy)[1,4′-bipiperidin]-1′-yl]-N-[(3,5-difluoropyridin-2-yl)methyl]-1,3-thiazole-5-carboxamide, 2-[(3R)-3-(cyclobutylmethoxy)[1,4′-bipiperidin]-1′-yl]-N-[(3,5-difluoropyridin-2-yl)methyl]-1,3-thiazole-5-carboxamide, formic acid-N-[(3,5-difluoropyridin-2-yl)methyl]-2-[3-(2-fluoroethyl)[1,4′-bipiperidin]-1′-yl]-1,3-thiazole-5-carboxamide, 2-([1,4′-bipiperidin]-1′-yl)-N-[(3,5-difluoropyridin-2-yl)methyl]-1,3-thiazole-5-carboxamide, N-[1-(3,5-difluoropyridin-2-yl)cyclopropyl]-2-[(3R)-3-methyl[1,4′-bipiperidin]-1′-yl]-1,3-thiazole-5-carboxamide, N-[(3,5-difluoropyridin-2-yl)methyl]-4-ethyl-2-[(3R)-3-methyl[1,4′-bipiperidin]-1′-yl]-1,3-thiazole-5-carboxamide, 2-[4-(3S)-(1,1-difluoro-5-azaspiro[2.5]octan-5-yl)piperidin-1-yl]-N-[(3,5-difluoropyridin-2-yl)methyl]-1,3-thiazole-5-carboxamide, 2-[4-(3R)-(1,1-difluoro-5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-(3-phenyl[1,4′-bipiperidin]-1′-yl)-1,3-thiazole-5-carboxamide, 2-[4-(1,1-difluoro-5-azaspiro[2.5]octan-5-yl)-3-fluoropiperidin-1-yl]-N-[(3,5-difluoropyridin-2-yl)methyl]-1,3-thiazole-5-carboxamide, 2-[4-(5-azaspiro[2.5]octan-5-yl)-3-fluoropiperidin-1-yl]-N-[(3,5-difluoropyridin-2-yl)methyl]-1,3-thiazole-5-carboxamide,
as compound of formula (I), and
Oxybutynin,
and the salts, solvates and solvates of the salts thereof.

A preferred embodiment of the present invention is directed to combinations of N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-[4-(3,4-dihydroisoquinolin-2(1H)-yl)piperidin-1-yl]-1,3-thiazole-5-carboxamide, 2-[3-(cyclopropylmethyl)[1,4′-bipiperidin]-1′-yl]-N-[(3,5-difluoropyridin-2-yl)methyl]-1,3-thiazole-5-carboxamide, 2-[3-(difluoromethyl)[1,4′-bipiperidin]-1′-yl]-N-[(3,5-difluoropyridin-2-yl)methyl]-1,3-thiazolo-5-carboxamide, N-[(3,5-difluoropyridin-2-yl)methyl]-2-[3-(trifluoromethyl)[1,4′-bipiperidin]-1′-yl]-1,3-thiazole-5-carboxamide, N-[(3,5-difluoropyridin-2-yl)methyl]-2-[3-(fluoromethyl)-[1,4′-bipiperidin]-1′-yl]-1,3-thiazole-5-carboxamide, 2-{3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-4-methyl-2-[(3R)-3-methyl[1,4′-bipiperidin]-1′-yl]-1,3-thiazole-5-carboxamide, N-[(3,5-difluoro-pyridin-2-yl)methyl]-4-methyl-2-[(3R)-3-methyl[1,4′-bipiperidin]-1′-yl]-1,3-thiazole-5-carboxamide, N-[(3,5-difluoropyridin-2-yl)methyl]-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-4-(trifluoromethyl)-1,3-thiazole-5-carboxamide, N-[(3,5-difluoropyridin-2-yl)methyl]-5-ethyl-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-1,3-oxazole-4-carboxamide, N-[(3,5-difluoropyridin-2-yl)methyl]-5-methyl-2-[(3R)-3-methyl[1,4′-bipiperidin]-1′-yl]-1,3-oxazole-4-carboxamide, N-[(3,5-difluoropyridin-2-yl)methyl]-2-[(3R)-3-methoxy[1,4′-bipiperidin]-1′-yl]-1,3-thiazole-5-carboxamide, 2-[3-(difluoromethoxy)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-(3-ethyl[1,4′-bipiperidin]-1′-yl)-1,3-thiazole-5-carboxamide, 2-[(3R)-3-methyl[1,4′-bipiperidin]-1′-yl]-N-{[4-(trifluoromethyl)pyridin-2-yl]methyl}-1,3-thiazole-5-carboxamide, 2-[(3R)-3-methyl[1,4′-bipiperidin]-1′-yl]-N-[3-(trifluoromethyl)benzyl]-1,3-thiazole-5-carboxamide, N-[(3-fluoropyridin-2-yl)methyl]-2-[(3R)-3-methyl[1,4′-bipiperidin]-1′-yl]-1,3-thiazole-5-carboxamide, N-(5-chloro-2-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[4-(trifluoromethyl)benzyl]-1,3-thiazole-5-carboxamide, N-[(5-chloro-3-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(3-methylpyridin-2-yl)methyl]-1,3-thiazole-5-carboxamide, 2-[(3R)-3-methyl[1,4′-bipiperidin]-1′-yl]-N-[(4-methylpyridin-2-yl)methyl]-1,3-thiazole-5-carboxamide, N-[(3-chloropyridin-2-yl)methyl]-2-[(3R)-3-methyl[1,4′-bipiperidin]-1′-yl]-1,3-thiazole-5-carboxamide, N-[(3-fluoropyridin-2-yl)methyl]-N-methyl-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethyl)pyridin-2-yl]methyl}-1,3-thiazole-5-carboxamide, N-[(5-chloropyridin-2-yl)methyl]-2-[(3R)-3-methyl[1,4′-bipiperidin]-1′-yl]-1,3-thiazole-5-carboxamide, N-[1-(2,5-difluorophenyl)ethyl]-2-[(3R)-3-methyl[1,4′-bipiperidin]-1′-yl]-1,3-thiazole-5-carboxamide, N-[(3-chloro-5-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethoxy)pyridin-2-yl]methyl}-1,3-thiazole-5-carboxamide, N-(4-chlorobenzyl)-2-[(3R)-3-methyl[1,4′-bipiperidin]-1′-yl]-1,3-thiazole-5-carboxamide, N-(2-chloro-5-fluorobenzyl)-2-[(3R)-3-methyl[1,4′-bipiperidin]-1′-yl]-1,3-thiazole-5-carboxamide, N-(4-methylbenzyl)-2-[(3R)-3-methyl[1,4′-bipiperidin]-1′-yl]-1,3-thiazole-5-carboxamide, N-(3-methylbenzyl)-2-[(3R)-3-methyl[1,4′-bipiperidin]-1′-yl]-1,3-thiazole-5-carboxamide, N-(2-methylbenzyl)-2-[(3R)-3-methyl[1,4′-bipiperidin]-1′-yl]-1,3-thiazole-5-carboxamide, 2-[(3S)-(difluoromethyl)[1,4′-bipiperidin]-1′-yl]-N-[(3,5-difluoropyridin-2-yl)methyl]-1,3-thiazole-5-carboxamide, 2-[(3R)-3-(difluoromethyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[(3S)-3-(fluoromethyl)[1,4′-bipiperidin]-1′-yl]-1,3-thiazole-5-carboxamide, N-[(3,5-difluoropyridin-2-yl)methyl]-2-[(3R)-3-(fluoromethyl)[1,4′-bipiperidin]-1′-yl]-1,3-thiazole-5-carboxamide, N-[(3,5-difluoropyridin-2-yl)methyl]-2-[(3S)-3-(trifluoromethyl)[1,4′-bipiperidin]-1′-yl]-1,3-thiazole-5-carboxamide, N-[(3,5-difluoropyridin-2-yl)methyl]-2-[(3R)-3-(trifluoromethyl)[1,4′-bipiperidin]-1′-yl]-1,3-thiazole-5-carboxamide, 2-{(3S)-3-[(3,3-difluorocyclobutyl)methoxy][1,4′-bipiperidin]-1′-yl}-N-[(3,5-difluoropyridin-2-yl)methyl]-1,3-thiazole-5-carboxamide, 2-{(3R)-3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(1S)-1-(2,5-difluorophenyl)ethyl]-2-[(3R)-3-methyl[1,4′-bipiperidin]-1′-yl]-1,3-thiazole-5-carboxamide, N-[(1R)-1-(2,5-difluorophenyl)ethyl]-2-[(3R)-3-methyl[1,4′-bipiperidin]-1′-yl]-1,3-thiazole-5-carboxamide, N-[(3,5-difluoropyridin-2-yl)methyl]-2-[3-(methoxymethyl)[1,4′-bipiperidin]-1′-yl]-1,3-thiazole-5-carboxamide, N-[(3,5-difluoropyridin-2-yl)methyl]-3-[(3R)-3-methyl[1,4′-bipiperidin]-1′-yl]-1,2,4-oxadiazole-5-carboxamide, N-[(3,5-difluoropyridin-2-yl)methyl]-2-[(3R)-3′-fluor-3-methyl[1,4′-bipiperidin]-1′-yl]-1,3-thiazole-5-carboxamide, ent-N-[(3,5-difluoropyridin-2-yl)methyl]-2-[(3R), (3′R)-3′-fluoro-3-methyl[1,4′-bipiperidin]-1′-yl]-1,3-thiazole-5-carboxamide, ent-N-[(3,5-difluoropyridin-2-yl)methyl]-2-[(3R), (3'S)-3′-fluoro-3-methyl[1,4′-bipiperidin]-1′-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[(5-fluoro-2-thienyl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-(pyridin-4-ylmethyl)-1,3-thiazole-5-carboxamide, N-[(3,5-difluoropyridin-2-yl)methyl]-2-{3-[(2,2,2-trifluoroethoxy)methyl][1,4′-bipiperidin]-1′-yl}-1,3-thiazole-5-carboxamide, N-[(3,5-difluoropyridin-2-yl)methyl]-2-[3-({[1-(fluoromethyl)cyclopropyl]methoxy}methyl)[1,4′-bipiperidin]-1′-yl]-1,3-thiazole-5-carboxamide, 2-[3-({[1-(difluoromethyl)cyclopropyl]methoxy}methyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[3-({[1-(trifluoromethyl)cyclopropyl]methoxy}methyl)[1,4′-bipiperidin]-1′-yl]-1,3-thiazole-5-carboxamide, N-[(3,5-difluoropyridin-2-yl)methyl]-2-(3,3-dimethyl[1,4′-bipiperidin]-1′-yl)-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, 2-[4-(1,1-difluoro-5-azaspiro[2.5]octan-5-yl)piperidin-1-yl]-N-[(3,5-difluoropyridin-2-yl)methyl]-1,3-thiazole-5-carboxamide, 2-[3-(cyclobutylmethoxy)[1,4′-bipiperidin]-1′-yl]-N-[(3,5-difluoropyridin-2-yl)methyl]-1,3-thiazole-5-carboxamide, 2-[3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclobutyloxy)methyl]-[1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[3-ethoxy[1,4′-bipiperidin]-1′-yl]-1,3-thiazole-5-carboxamide, N-[(3,5-difluoropyridin-2-yl)methyl]-2-{4-[(3R)-3-methylpiperidin-1-yl]azepan-1-yl}-1,3-thiazole-5-carboxamide, 2-[(3R)-3-methyl[1,4′-bipiperidin]-1′-yl]-N-[(6-methylpyridin-3-yl)methyl]-1,3-thiazole-5-carboxamide, N-benzyl-2-[(3R)-3-methyl[1,4′-bipiperidin]-1′-yl]-1,3-thiazole-5-carboxamide, N-[(3,5-difluoropyridin-2-yl)methyl]-2-[3-({[3-fluorobutyl]oxy}methyl)[1,4′-bipiperidin]-1′-yl]-1,3-thiazole-5-carboxamide, 2-(3-{[(3,3-difluorocyclobutyl)methoxy]methyl}[1,4′-bipiperidin]-1′-yl)-N-[(3,5-difluoropyridin-2-yl)methyl]-1,3-thiazole-5-carboxamide, N-[(3-fluoropyridin-4-yl)methyl]-2-[(3R)-3-methyl[1,4′-bipiperidin]-1′-yl]-1,3-thiazole-5-carboxamide, N-[(3,5-difluoropyridin-2-yl)methyl]-2-[3-(2,2,2-trifluoroethoxy)[1,4′-bipiperidin]-1′-yl]-1,3-thiazole-5-carboxamide, N-[(4,6-dimethylpyridin-3-yl)methyl]-2-[(3R)-3-methyl[1,4′-bipiperidin]-1′-yl]-1,3-thiazole-5-carboxamide, N-[(4-chloro-1-methyl-1H-pyrazol-5-yl)methyl]-2-[(3R)-3-methyl[1,4′-bipiperidin]-1′-yl]-1,3-thiazole-5-carboxamide, N-(3-methoxybenzyl)-2-[(3R)-3-methyl[1,4′-bipiperidin]-1′-yl]-1,3-thiazole-5-carboxamide, N-(2,5-difluorobenzyl)-2-[(3R)-3-methyl[1,4′-bipiperidin]-1′-yl]-1,3-thiazole-5-carboxamide, N-(3-hydroxybenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(2R)-2-phenylpropyl]-1,3-thiazole-5-carboxamide, N-(4-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-(pyridin-3-ylmethyl)-1,3-thiazole-5-carboxamide, N-(3-fluorobenzyl)-2-[(3R)-3-methyl[1,4′-bipiperidin]-1′-yl]-1,3-thiazole-5-carboxamide, N-(2-fluorobenzyl)-2-[(3R)-3-methyl[1,4′-bipiperidin]-1′-yl]-1,3-thiazole-5-carboxamide, N-(2-chloro-4-fluorophenyl)-2-[(3R)-3-methyl[1,4′-bipiperidin]-1′-yl]-1,3-thiazole-5-carboxamide, N-(3-cyano-4-fluorophenyl)-2-[(3R)-3-methyl[1,4′-bipiperidin]-1′-yl]-1,3-thiazole-5-carboxamide, N-methyl-2-[(3R)-3-methyl[1,4′-bipiperidin]-1′-yl]-N-(pyridin-3-ylmethyl)-1,3-thiazole-5-carboxamide, N-methyl-2-[(3R)-3-methyl[1,4′-bipiperidin]-1′-yl]-N-(pyridin-4-ylmethyl)-1,3-thiazole-5-carboxamide, N-benzyl-N-methyl-2-[(3R)-3-methyl[1,4′-bipiperidin]-1′-yl]-1,3-thiazole-5-carboxamide, N-(2-cyclopropylphenyl)-2-[(3R)-3-methyl[1,4′-bipiperidin]-1′-yl]-1,3-thiazole-5-carboxamide, N-(3-chlorobenzyl)-2-[(3R)-3-methyl[1,4′-bipiperidin]-1′-yl]-1,3-thiazol-5-carboxamide, 2-[(3R)-3-methyl[1,4′-bipiperidin]-1′-yl]-N-[(1R)-1-(4-methylphenyl)ethyl]-1,3-thiazole-5-carboxamide, N-(2-ethylpyridin-4-yl)-2-[(3R)-3-methyl[1,4′-bipiperidin]-1′-yl]-1,3-thiazole-5-carboxamide, N-[(3,5-difluoropyridin-2-yl)methyl]-2-[(3S)-3-(methoxymethyl)[1,4′-bipiperidin]-1′-yl]-1,3-thiazole-5-carboxamide, N-[(3,5-difluoropyridin-2-yl)methyl]-2-[(3R)-3-(methoxymethyl) [1,4′-bipiperidin]-1′-yl]-1,3-thiazole-5-carboxamide, 2-{(3S)-3-[(cyclobutyloxy)methyl][1,4′-bipiperidin]-1′-yl}-N-[(3,5-difluoropyridin-2-yl)methyl]-1,3-thiazole-5-carboxamide, 2-{(3R)-3-[(cyclobutyloxy)methyl][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-(3-isopropyl[1,4′-bipiperidin]-1′-yl)-1,3-thiazole-5-carboxamide, N-[(3,5-difluoropyridin-2-yl)methyl]-2-[4-((4S)-4-methylazepan-1-yl)piperidin-1-yl]-1,3-thiazole-5-carboxamide, N-[(3,5-difluoropyridin-2-yl)methyl]-2-[4-((4R)-4-methylazepan-1-yl)piperidin-1-yl]-1,3-thiazole-5-carboxamide, N-[(3,5-difluoropyridin-2-yl)methyl]-2-{(3S)-3-[(2,2,2-trifluoroethoxy)methyl][1,4′-bipiperidin]-1′-yl}-1,3-thiazole-5-carboxamide, N-[(3,5-difluoropyridin-2-yl)methyl]-2-{(3R)-3-[(2,2,2-trifluoroethoxy)methyl][1,4′-bipiperidin]-1′-yl}-1,3-thiazole-5-carboxamide, 2-{3-[(2,2-difluorocyclopropyl)methoxy][1,4′-bipiperidin]-1′-yl}-N-[(3,5-difluoropyridin-2-yl)methyl]-1,3-thiazole-5-carboxamide, 2-[3-(cyclobutyloxy)[1,4′-bipiperidin]-1′-yl]-N-[(3,5-difluoropyridin-2-yl)methyl]-1,3-thiazole-5-carboxamide, 2-{3-[(3,3-difluorocyclobutyl)oxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[(3R)-3′-fluoro-3-methyl[1,4′-bipiperidin]-1′-yl]-1,3-thiazole-4-carboxamide, N-[(3,5-difluoropyridin-2-yl)methyl]-2-[(3R)-3′-fluoro-3-methyl[1,4′-bipiperidin]-1′-yl]-1,3-oxazole-4-carboxamide, N-(5-chloro-2-fluorobenzyl)-2-[(3R)-3′-fluoro-3-methyl[1,4′-bipiperidin]-1′-yl]-1,3-thiazole-5-carboxamide, 2-[(3R)-3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[1-(2,5-difluorophenyl)ethyl]-2-[(3R)-3′-fluoro-3-methyl[1,4′-bipiperidin]-1′-yl]-1,3-thiazole-5-carboxamide, 4-(2-chlorophenyl)-N-[(3,5-difluoropyridin-2-yl)methyl]-2-[(3R)-3-methyl[1,4′-bipiperidin]-1′-yl]-1,3-thiazole-5-carboxamide, 4-bromo-N-[(3,5-difluoropyridin-2-yl)methyl]-2-[(3R)-3-methyl[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-(3-propyl[1,4′-bipiperidin]-1′-yl)-1,3-thiazole-5-carboxamide, 4-cyclopropyl-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-((3S)-3-ethoxy[1,4′-bipiperidin]-1′-yl)-1,3-thiazole-5-carboxamide, N-[(3,5-difluoropyridin-2-yl)methyl]-2-((3R)-3-ethoxy[1,4′-bipiperidin]-1′-yl)-1,3-thiazole-5-carboxamide, 2-[(3S)-3-(cyclobutylmethoxy)[1,4′-bipiperidin]-1′-yl]-N-[(3,5-difluoropyridin-2-yl)methyl]-1,3-thiazole-5-carboxamide, 2-[(3R)-3-(cyclobutylmethoxy)[1,4′-bipiperidin]-1′-yl]-N-[(3,5-difluoropyridin-2-yl)methyl]-1,3-thiazole-5-carboxamide, formic acid-N-[(3,5-difluoropyridin-2-yl)methyl]-2-[3-(2-fluoroethyl)[1,4′-bipiperidin]-1′-yl]-1,3-thiazole-5-carboxamide, 2-([1,4′-bipiperidin]-1′-yl)-N-[(3,5-difluoropyridin-2-yl)methyl]-1,3-thiazole-5-carboxamide, N-[1-(3,5-difluoropyridin-2-yl)cyclopropyl]-2-[(3R)-3-methyl[1,4′-bipiperidin]-1′-yl]-1,3-thiazole-5-carboxamide, N-[(3,5-difluoropyridin-2-yl)methyl]-4-ethyl-2-[(3R)-3-methyl[1,4′-bipiperidin]-1′-yl]-1,3-thiazole-5-carboxamide, 2-[4-(3S)-(1,1-difluoro-5-azaspiro[2.5]octan-5-yl)piperidin-1-yl]-N-[(3,5-difluoropyridin-2-yl)methyl]-1,3-thiazole-5-carboxamide, 2-[4-(3R)-(1,1-difluoro-5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-(3-phenyl[1,4′-bipiperidin]-1′-yl)-1,3-thiazole-5-carboxamide, 2-[4-(1,1-difluoro-5-azaspiro[2.5]octan-5-yl)-3-fluoropiperidin-1-yl]-N-[(3,5-difluoropyridin-2-yl)methyl]-1,3-thiazole-5-carboxamide, 2-[4-(5-azaspiro[2.5]octan-5-yl)-3-fluoropiperidin-1-yl]-N-[(3,5-difluoropyridin-2-yl)methyl]-1,3-thiazole-5-carboxamide,
as compound of formula (I), and
R-Oxybutynin,
and the salts, solvates and solvates of the salts thereof.

A preferred embodiment of the present invention is directed to combinations of N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-[4-(3,4-dihydroisoquinolin-2(1H)-yl)piperidin-1-yl]-1,3-thiazole-5-carboxamide, 2-[3-(cyclopropylmethyl)[1,4′-bipiperidin]-1′-yl]-N-[(3,5-difluoropyridin-2-yl)methyl]-1,3-thiazole-5-carboxamide, 2-[3-(difluoromethyl)[1,4′-bipiperidin]-1′-yl]-N-[(3,5-difluoropyridin-2-yl)methyl]-1,3-thiazolo-5-carboxamide, N-[(3,5-difluoropyridin-2-yl)methyl]-2-[3-(trifluoromethyl)[1,4′-bipiperidin]-1′-yl]-1,3-thiazole-5-carboxamide, N-[(3,5-difluoropyridin-2-yl)methyl]-2-[3-(fluoromethyl)-[1,4′-bipiperidin]-1′-yl]-1,3-thiazole-5-carboxamide, 2-{3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-4-methyl-2-[(3R)-3-methyl[1,4′-bipiperidin]-1′-yl]-1,3-thiazole-5-carboxamide, N-[(3,5-difluoro-pyridin-2-yl)methyl]-4-methyl-2-[(3R)-3-methyl[1,4′-bipiperidin]-1′-yl]-1,3-thiazole-5-carboxamide, N-[(3,5-difluoropyridin-2-yl)methyl]-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-4-(trifluoromethyl)-1,3-thiazole-5-carboxamide, N-[(3,5-difluoropyridin-2-yl)methyl]-5-ethyl-2-[(3R)-3-methyl[1,4′-bipiperidin]-1′-yl]-1,3-thiazole-4-carboxamide, N-[(3,5-difluoropyridin-2-yl)methyl]-2-[(3R)-3-methyl[1,4′-bipiperidin]-1′-yl]-1,3-oxazole-4-carboxamide, N-[(3,5-difluoropyridin-2-yl)methyl]-5-methyl-2-[(3R)-3-methyl[1,4′-bipiperidin]-1′-yl]-1,3-oxazole-4-carboxamide, N-[(3,5-difluoropyridin-2-yl)methyl]-2-[(3R)-3-methoxy[1,4′-bipiperidin]-1′-yl]-1,3-thiazole-5-carboxamide, 2-[3-(difluoromethoxy)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-(3-ethyl[1,4′-bipiperidin]-1′-yl)-1,3-thiazole-5-carboxamide, 2-[(3R)-3-methyl[1,4′-bipiperidin]-1′-yl]-N-{[4-(trifluoromethyl)pyridin-2-yl]methyl}-1,3-thiazole-5-carboxamide, 2-[(3R)-3-methyl[1,4′-bipiperidin]-1′-yl]-N-[3-(trifluoromethyl)benzyl]-1,3-thiazole-5-carboxamide, N-[(3-fluoropyridin-2-yl)methyl]-2-[(3R)-3-methyl[1,4′-bipiperidin]-1′-yl]-1,3-thiazole-5-carboxamide, N-(5-chloro-2-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[4-(trifluoromethyl)benzyl]-1,3-thiazole-5-carboxamide, N-[(5-chloro-3-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(3-methylpyridin-2-yl)methyl]-1,3-thiazole-5-carboxamide, 2-[(3R)-3-methyl[1,4′-bipiperidin]-1′-yl]-N-[(4-methylpyridin-2-yl)methyl]-1,3-thiazole-5-carboxamide, N-[(3-chloropyridin-2-yl)methyl]-2-[(3R)-3-methyl[1,4′-bipiperidin]-1′-yl]-1,3-thiazole-5-carboxamide, N-[(3-fluoropyridin-2-yl)methyl]-N-methyl-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethyl)pyridin-2-yl]methyl}-1,3-thiazole-5-carboxamide, N-[(5-chloropyridin-2-yl)methyl]-2-[(3R)-3-methyl[1,4′-bipiperidin]-1′-yl]-1,3-thiazole-5-carboxamide, N-[1-(2,5-difluorophenyl)ethyl]-2-[(3R)-3-methyl[1,4′-bipiperidin]-1′-yl]-1,3-thiazole-5-carboxamide, N-[(3-chloro-5-fluoropyridin-2-yl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-{[6-(trifluoromethoxy)pyridin-2-yl]methyl}-1,3-thiazole-5-carboxamide, N-(4-chlorobenzyl)-2-[(3R)-3-methyl[1,4′-bipiperidin]-1′-yl]-1,3-thiazole-5-carboxamide, N-(2-chloro-5-fluorobenzyl)-2-[(3R)-3-methyl[1,4′-bipiperidin]-1′-yl]-1,3-thiazole-5-carboxamide, N-(4-methylbenzyl)-2-[(3R)-3-methyl[1,4′-bipiperidin]-1′-yl]-1,3-thiazole-5-carboxamide, N-(3-methylbenzyl)-2-[(3R)-3-methyl[1,4′-bipiperidin]-1′-yl]-1,3-thiazole-5-carboxamide, N-(2-methylbenzyl)-2-[(3R)-3-methyl[1,4′-bipiperidin]-1′-yl]-1,3-thiazole-5-carboxamide, 2-[(3S)-(difluoromethyl)[1,4′-bipiperidin]-1′-yl]-N-[(3,5-difluoropyridin-2-yl)methyl]-1,3-thiazole-5-carboxamide, 2-[(3R)-3-(difluoromethyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[(3S)-3-(fluoromethyl)[1,4′-bipiperidin]-1′-yl]-1,3-thiazole-5-carboxamide, N-[(3,5-difluoropyridin-2-yl)methyl]-2-[(3R)-3-(fluoromethyl)[1,4′-bipiperidin]-1′-yl]-1,3-thiazole-5-carboxamide, N-[(3,5-difluoropyridin-2-yl)methyl]-2-[(3S)-3-(trifluoromethyl)[1,4′-bipiperidin]-1′-yl]-1,3-thiazole-5-carboxamide, N-[(3,5-difluoropyridin-2-yl)methyl]-2-[(3R)-3-(trifluoromethyl)[1,4′-bipiperidin]-1′-yl]-1,3-thiazole-5-carboxamide, 2-{(3S)-3-[(3,3-difluorocyclobutyl)methoxy][1,4′-bipiperidin]-1′-yl}-N-[(3,5-difluoropyridin-2-yl)methyl]-1,3-thiazole-5-carboxamide, 2-{(3R)-3-[(3,3-difluorocyclobutyl)methoxy][1,4′-bipiperidin]-1′-yl}-N-[(3,5-difluoropyridin-2-yl)methyl]-1,3-thiazole-5-carboxamide, N-[(1S)-1-(2,5-difluorophenyl)ethyl]-2-[(3R)-3-methyl[1,4′-bipiperidin]-1′-yl]-1,3-thiazole-5-carboxamide, N-[(1R)-1-(2,5-difluorophenyl)ethyl]-2-[(3R)-3-methyl[1,4′-bipiperidin]-1′-yl]-1,3-thiazole-5-carboxamide, N-[(3,5-difluoropyridin-2-yl)methyl]-2-[3-(methoxymethyl)[1,4′-bipiperidin]-1′-yl]-1,3-thiazole-5-carboxamide, N-[(3,5-difluoropyridin-2-yl)methyl]-3-[(3R)-3-methyl[1,4′-bipiperidin]-1′-yl]-1,2,4-oxadiazole-5-carboxamide, N-[(3,5-difluoropyridin-2-yl)methyl]-2-[(3R)-3′-fluor-3-methyl[1,4′-bipiperidin]-1′-yl]-1,3-thiazole-5-carboxamide, ent-N-[(3,5-difluoropyridin-2-yl)methyl]-2-[(3R), (3′R)-3′-fluoro-3-methyl[1,4′-bipiperidin]-1′-yl]-1,3-thiazole-5-carboxamide, ent-N-[(3,5-difluoropyridin-2-yl)methyl]-2-[(3R), (3'S)-3′-fluoro-3-methyl[1,4′-bipiperidin]-1′-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[(3,5-difluoropyridin-2-yl)methyl]-2-[4-(4-methylazepan-1-yl)piperidin-1-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[1-(3,5-difluoropyridin-2-yl)ethyl]-2-[(3R)-3-methyl[1,4′-bipiperidin]-1′-yl]-1,3-thiazole-5-carboxamide, N-[(5-fluoro-2-thienyl)methyl]-2-[(3R)-3-methyl[1,4′-bipiperidin]-1′-yl]-1,3-thiazole-5-carboxamide, 2-[(3R)-3-methyl[1,4′-bipiperidin]-1′-yl]-N-(pyridin-4-ylmethyl)-1,3-thiazole-5-carboxamide, N-[(3,5-difluoropyridin-2-yl)methyl]-2-{3-[(2,2,2-trifluoroethoxy)methyl][1,4′-bipiperidin]-1′-yl}-1,3-thiazole-5-carboxamide, N-[(3,5-difluoropyridin-2-yl)methyl]-2-[3-({[1-(fluoromethyl)cyclopropyl]methoxy}methyl)[1,4′-bipiperidin]-1′-yl]-1,3-thiazole-5-carboxamide, 2-[3-({[1-(difluoromethyl)cyclopropyl]methoxy}methyl)[1,4′-bipiperidin]-1′-yl]-N-[(3,5-difluoropyridin-2-yl)methyl]-1,3-thiazole-5-carboxamide, N-[(3,5-difluoropyridin-2-yl)methyl]-2-[3-({[1-(trifluoromethyl)cyclopropyl]methoxy}methyl)[1,4′-bipiperidin]-1′-yl]-1,3-thiazole-5-carboxamide, N-[(3,5-difluoropyridin-2-yl)methyl]-2-(3,3-dimethyl[1,4′-bipiperidin]-1′-yl)-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, 2-[4-(1,1-difluoro-5-azaspiro[2.5]octan-5-yl)piperidin-1-yl]-N-[(3,5-difluoropyridin-2-yl)methyl]-1,3-thiazole-5-carboxamide, 2-[3-(cyclobutylmethoxy)[1,4′-bipiperidin]-1′-yl]-N-[(3,5-difluoropyridin-2-yl)methyl]-1,3-thiazole-5-carboxamide, 2-[3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclobutyloxy)methyl]-[1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[3-ethoxy[1,4′-bipiperidin]-1′-yl]-1,3-thiazole-5-carboxamide, N-[(3,5-difluoropyridin-2-yl)methyl]-2-{4-[(3R)-3-methylpiperidin-1-yl]azepan-1-yl}-1,3-thiazole-5-carboxamide, 2-[(3R)-3-methyl[1,4′-bipiperidin]-1′-yl]-N-[(6-methylpyridin-3-yl)methyl]-1,3-thiazole-5-carboxamide, N-benzyl-2-[(3R)-3-methyl[1,4′-bipiperidin]-1′-yl]-1,3-thiazole-5-carboxamide, N-[(3,5-difluoropyridin-2-yl)methyl]-2-[3-({[3-fluorobutyl]oxy}methyl)[1,4′-bipiperidin]-1′-yl]-1,3-thiazole-5-carboxamide, 2-(3-{[(3,3-difluorocyclobutyl)methoxy]methyl}[1,4′-bipiperidin]-1′-yl)-N-[(3,5-difluoropyridin-2-yl)methyl]-1,3-thiazole-5-carboxamide, N-[(3-fluoropyridin-4-yl)methyl]-2-[(3R)-3-methyl[1,4′-bipiperidin]-1′-yl]-1,3-thiazole-5-carboxamide, N-[(3,5-difluoropyridin-2-yl)methyl]-2-[3-(2,2,2-trifluoroethoxy)[1,4′-bipiperidin]-1′-yl]-1,3-thiazole-5-carboxamide, N-[(4,6-dimethylpyridin-3-yl)methyl]-2-[(3R)-3-methyl[1,4′-bipiperidin]-1′-yl]-1,3-thiazole-5-carboxamide, N-[(4-chloro-1-methyl-1H-pyrazol-5-yl)methyl]-2-[(3R)-3-methyl[1,4′-bipiperidin]-1′-yl]-1,3-thiazole-5-carboxamide, N-(3-methoxybenzyl)-2-[(3R)-3-methyl[1,4′-bipiperidin]-1′-yl]-1,3-thiazole-5-carboxamide, N-(2,5-difluorobenzyl)-2-[(3R)-3-methyl[1,4′-bipiperidin]-1′-yl]-1,3-thiazole-5-carboxamide, N-(3-hydroxybenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-[(2R)-2-phenylpropyl]-1,3-thiazole-5-carboxamide, N-(4-fluorobenzyl)-2-[(3R)-3-methyl[1,4′-bipiperidin]-1′-yl]-1,3-thiazole-5-carboxamide, 2-[(3R)-3-methyl[1,4′-bipiperidin]-1′-yl]-N-(pyridin-3-ylmethyl)-1,3-thiazole-5-carboxamide, N-(3-fluorobenzyl)-2-[(3R)-3-methyl[1,4′-bipiperidin]-1′-yl]-1,3-thiazole-5-carboxamide, N-(2-fluorobenzyl)-2-[(3R)-3-methyl[1,4′-bipiperidin]-1′-yl]-1,3-thiazole-5-carboxamide, N-(2-chloro-4-fluorophenyl)-2-[(3R)-3-methyl[1,4′-bipiperidin]-1′-yl]-1,3-thiazole-5-carboxamide, N-(3-cyano-4-fluorophenyl)-2-[(3R)-3-methyl[1,4′-bipiperidin]-1′-yl]-1,3-thiazole-5-carboxamide, N-methyl-2-[(3R)-3-methyl[1,4′-bipiperidin]-1′-yl]-N-(pyridin-3-ylmethyl)-1,3-thiazole-5-carboxamide, N-methyl-2-[(3R)-3-methyl[1,4′-bipiperidin]-1′-yl]-N-(pyridin-4-ylmethyl)-1,3-thiazole-5-carboxamide, N-benzyl-N-methyl-2-[(3R)-3-methyl[1,4′-bipiperidin]-1′-yl]-1,3-thiazole-5-carboxamide, N-(2-cyclopropylphenyl)-2-[(3R)-3-methyl[1,4′-bipiperidin]-1′-yl]-1,3-thiazole-5-carboxamide, N-(3-chlorobenzyl)-2-[(3R)-3-methyl[1,4′-bipiperidin]-1′-yl]-1,3-thiazol-5-carboxamide, 2-[(3R)-3-methyl[1,4′-bipiperidin]-1′-yl]-N-[(1R)-1-(4-methylphenyl)ethyl]-1,3-thiazole-5-carboxamide, N-(2-ethylpyridin-4-yl)-2-[(3R)-3-methyl[1,4′-bipiperidin]-1′-yl]-1,3-thiazole-5-carboxamide, N-[(3,5-difluoropyridin-2-yl)methyl]-2-[(3S)-3-(methoxymethyl)[1,4′-bipiperidin]-1′-yl]-1,3-thiazole-5-carboxamide, N-[(3,5-difluoropyridin-2-yl)methyl]-2-[(3R)-3-(methoxymethyl) [1,4′-bipiperidin]-1′-yl]-1,3-thiazole-5-carboxamide, 2-{(3S)-3-[(cyclobutyloxy)methyl][1,4′-bipiperidin]-1′-yl}-N-[(3,5-difluoropyridin-2-yl)methyl]-1,3-thiazole-5-carboxamide, 2-{(3R)-3-[(cyclobutyloxy)methyl][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-(3-isopropyl[1,4′-bipiperidin]-1′-yl)-1,3-thiazole-5-carboxamide, N-[(3,5-difluoropyridin-2-yl)methyl]-2-[4-((4S)-4-methylazepan-1-yl)piperidin-1-yl]-1,3-thiazole-5-carboxamide, N-[(3,5-difluoropyridin-2-yl)methyl]-2-[4-((4R)-4-methylazepan-1-yl)piperidin-1-yl]-1,3-thiazole-5-carboxamide, N-[(3,5-difluoropyridin-2-yl)methyl]-2-{(3S)-3-[(2,2,2-trifluoroethoxy)methyl][1,4′-bipiperidin]-1′-yl}-1,3-thiazole-5-carboxamide, N-[(3,5-difluoropyridin-2-yl)methyl]-2-{(3R)-3-[(2,2,2-trifluoroethoxy)methyl][1,4′-bipiperidin]-1′-yl}-1,3-thiazole-5-carboxamide, 2-{3-[(2,2-difluorocyclopropyl)methoxy][1,4′-bipiperidin]-1′-yl}-N-[(3,5-difluoropyridin-2-yl)methyl]-1,3-thiazole-5-carboxamide, 2-[3-(cyclobutyloxy)[1,4′-bipiperidin]-1′-yl]-N-[(3,5-difluoropyridin-2-yl)methyl]-1,3-thiazole-5-carboxamide, 2-{3-[(3,3-difluorocyclobutyl)oxy][1,4′-bipiperidin]-1′-yl}-N-[(3,5-difluoropyridin-2-yl)methyl]-1,3-thiazole-5-carboxamide, N-[(3,5-difluoropyridin-2-yl)methyl]-2-[(3R)-3′-fluoro-3-methyl[1,4′-bipiperidin]-1′-yl]-1,3-thiazole-4-carboxamide, N-[(3,5-difluoropyridin-2-yl)methyl]-2-[(3R)-3′-fluoro-3-methyl[1,4′-bipiperidin]-1′-yl]-1,3-oxazole-4-carboxamide, N-(5-chloro-2-fluorobenzyl)-2-[(3R)-3′-fluoro-3-methyl[1,4′-bipiperidin]-1′-yl]-1,3-thiazole-5-carboxamide, 2-[(3R)-3-(cyclopropylmethoxy)[1,4′-bipiperidin]-1′-yl]-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, 2-{3-[(cyclopropylmethoxy)methyl][1,4′-bipiperidin]-1′-yl}-N-[(3,5-difluoropyridin-2-yl)methyl]-1,3-thiazole-5-carboxamide, N-[1-(2,5-difluorophenyl)ethyl]-2-[(3R)-3′-fluoro-3-methyl[1,4′-bipiperidin]-1′-yl]-1,3-thiazole-5-carboxamide, 4-(2-chlorophenyl)-N-[(3,5-difluoropyridin-2-yl)methyl]-2-[(3R)-3-methyl[1,4′-bipiperidin]-1′-yl]-1,3-thiazole-5-carboxamide, 4-bromo-N-[(3,5-difluoropyridin-2-yl)methyl]-2-[(3R)-3-methyl[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-(3-propyl[1,4′-bipiperidin]-1′-yl)-1,3-thiazole-5-carboxamide, 4-cyclopropyl-N-[(3,5-difluoropyridin-2-yl)methyl]-2-[(3R)-3-methyl[1,4′-bipiperidin]-1′-yl]-1,3-thiazole-5-carboxamide, N-[(3,5-difluoropyridin-2-yl)methyl]-2-((3S)-3-ethoxy[1,4′-bipiperidin]-1′-yl)-1,3-thiazole-5-carboxamide, N-[(3,5-difluoropyridin-2-yl)methyl]-2-((3R)-3-ethoxy[1,4′-bipiperidin]-1′-yl)-1,3-thiazole-5-carboxamide, 2-[(3S)-3-(cyclobutylmethoxy)[1,4′-bipiperidin]-1′-yl]-N-[(3,5-difluoropyridin-2-yl)methyl]-1,3-thiazole-5-carboxamide, 2-[(3R)-3-(cyclobutylmethoxy)[1,4′-bipiperidin]-1′-yl]-N-[(3,5-difluoropyridin-2-yl)methyl]-1,3-thiazole-5-carboxamide, formic acid-N-[(3,5-difluoropyridin-2-yl)methyl]-2-[3-(2-fluoroethyl)[1,4′-bipiperidin]-1′-yl]-1,3-thiazole-5-carboxamide, 2-([1,4′-bipiperidin]-1′-yl)-N-[(3,5-difluoropyridin-2-yl)methyl]-1,3-thiazole-5-carboxamide, N-[1-(3,5-difluoropyridin-2-yl)cyclopropyl]-2-[(3R)-3-methyl[1,4′-bipiperidin]-1′-yl]-1,3-thiazole-5-carboxamide, N-[(3,5-difluoropyridin-2-yl)methyl]-4-ethyl-2-[(3R)-3-methyl[1,4′-bipiperidin]-1′-yl]-1,3-thiazole-5-carboxamide, 2-[4-(3S)-(1,1-difluoro-5-azaspiro[2.5]octan-5-yl)piperidin-1-yl]-N-[(3,5-difluoropyridin-2-yl)methyl]-1,3-thiazole-5-carboxamide, 2-[4-(3R)-(1,1-difluoro-5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-(3-phenyl[1,4′-bipiperidin]-1′-yl)-1,3-thiazole-5-carboxamide, 2-[4-(1,1-difluoro-5-azaspiro[2.5]octan-5-yl)-3-fluoropiperidin-1-yl]-N-[(3,5-difluoropyridin-2-yl)methyl]-1,3-thiazole-5-carboxamide, 2-[4-(5-azaspiro[2.5]octan-5-yl)-3-fluoropiperidin-1-yl]-N-[(3,5-difluoropyridin-2-yl)methyl]-1,3-thiazole-5-carboxamide,
as compound of formula (I), and
Tolterodine,
and the salts, solvates and solvates of the salts thereof.

Another preferred embodiment of the present invention is directed to combinations of compounds of formula (I) which are selected from the group consisting of
N-[(3,5-difluoropyridin-2-yl)methyl]-2-[(3R)-3-methyl[1,4′-bipiperidin]-1′-yl]-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-[(3R*)-3-(methoxymethyl)[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide and N-[1-(3,5-difluoropyridin-2-yl)cyclopropyl]-2-[(3R)-3-methyl[1,4′-bipiperidin]-1′-yl]-1,3-thiazole-5-carboxamide,
and muscarinic receptor antagonists,
and the salts, solvates and solvates of the salts thereof.

Another preferred embodiment of the present invention is directed to combinations of compounds of formula (I) which are selected from the group consisting of
N-[(3,5-difluoropyridin-2-yl)methyl]-2-[(3R)-3-methyl[1,4′-bipiperidin]-1′-yl]-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-[(3R*)-3-(methoxymethyl)[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide and N-[1-(3,5-difluoropyridin-2-yl)cyclopropyl]-2-[(3R)-3-methyl[1,4′-bipiperidin]-1′-yl]-1,3-thiazole-5-carboxamide and
muscarinic receptor antagonists selected from the group comprising Oxybutynin, R-Oxybutynin and Tolterodine,
and the salts, solvates and solvates of the salts thereof.

Another preferred embodiment of the present invention is directed to combinations of compounds of formula (I) which are selected from the group consisting of
N-[(3,5-difluoropyridin-2-yl)methyl]-2-[(3R)-3-methyl[1,4′-bipiperidin]-1′-yl]-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-[(3R*)-3-(methoxymethyl)[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide and N-[1-(3,5-difluoropyridin-2-yl)cyclopropyl]-2-[(3R)-3-methyl[1,4′-bipiperidin]-1′-yl]-1,3-thiazole-5-carboxamide and
Oxybutynin,
and the salts, solvates and solvates of the salts thereof.

Another preferred embodiment of the present invention is directed to combinations of compounds of formula (I) which are selected from the group consisting of
N-[(3,5-difluoropyridin-2-yl)methyl]-2-[(3R)-3-methyl[1,4′-bipiperidin]-1′-yl]-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-[(3R*)-3-(methoxymethyl)[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide and N-[1-(3,5-difluoropyridin-2-yl)cyclopropyl]-2-[(3R)-3-methyl[1,4′-bipiperidin]-1′-yl]-1,3-thiazole-5-carboxamide and
R-Oxybutynin,
and the salts, solvates and solvates of the salts thereof.

Another preferred embodiment of the present invention is directed to combinations of compounds of formula (I) which are selected from the group consisting of
N-[(3,5-difluoropyridin-2-yl)methyl]-2-[(3R)-3-methyl[1,4′-bipiperidin]-1′-yl]-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-[(3R*)-3-(methoxymethyl)[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide and N-[1-(3,5-difluoropyridin-2-yl)cyclopropyl]-2-[(3R)-3-methyl[1,4′-bipiperidin]-1′-yl]-1,3-thiazole-5-carboxamide and
Tolterodine,
and the salts, solvates and solvates of the salts thereof.

Another preferred embodiment of the present invention is directed to combinations of
N-[(3,5-difluoropyridin-2-yl)methyl]-2-[(3R)-3-methyl[1,4′-bipiperidin]-1′-yl]-1,3-thiazole-5-carboxamide, and
muscarinic receptor antagonists,
and the salts, solvates and solvates of the salts thereof.

Another preferred embodiment of the present invention is directed to combinations of
N-[(3,5-difluoropyridin-2-yl)methyl]-2-[(3R)-3-methyl[1,4′-bipiperidin]-1′-yl]-1,3-thiazole-5-carboxamide, and muscarinic receptor antagonists selected from the group comprising Oxybutynin, R-Oxybutynin and
Tolterodine,
and the salts, solvates and solvates of the salts thereof.

Another preferred embodiment of the present invention is directed to combinations of
N-[(3,5-difluoropyridin-2-yl)methyl]-2-[(3R)-3-methyl[1,4′-bipiperidin]-1′-yl]-1,3-thiazole-5-carboxamide,
and Oxybutynin,
and the salts, solvates and solvates of the salts thereof.

An another preferred embodiment of the present invention is directed to combinations of
N-[(3,5-difluoropyridin-2-yl)methyl]-2-[(3R)-3-methyl[1,4′-bipiperidin]-1′-yl]-1,3-thiazole-5-carboxamide, And R-Oxybutynin,
and the salts, solvates and solvates of the salts thereof.

Another preferred embodiment of the present invention is directed to combinations of
N-[(3,5-difluoropyridin-2-yl)methyl]-2-[(3R)-3-methyl[1,4′-bipiperidin]-1′-yl]-1,3-thiazole-5-carboxamide, and Tolterodine,
and the salts, solvates and solvates of the salts thereof.

In a possible subgroup of the compounds of formula I
X, Y and Z are selected such that the aromatic 5-membered ring has the structural formula h), i), j), k) or (r);

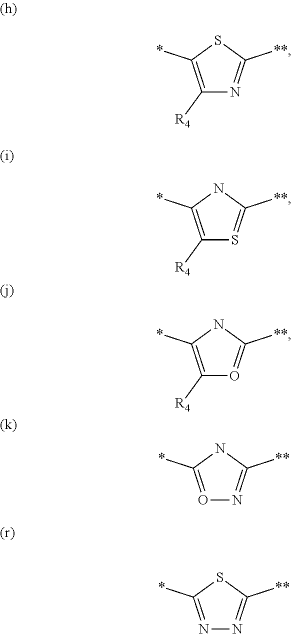

where * marks the attachment to the carbonyl group and ** marks the attachment to the nitrogen atom of the adjacent piperidine ring and
R₄ represents hydrogen, methyl, ethyl, cyclopropyl, trifluoromethyl, bromine, chlorine, phenyl;
   where phenyl may be substituted by chlorine,
and the salts, solvates and solvates of the salts thereof.

In a possible subgroup of the compounds of formula I
X, Y and Z are selected such that the aromatic 5-membered ring has the structural formula (h) or i);

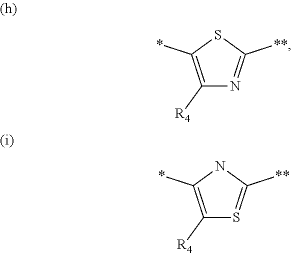

where * marks the attachment to the carbonyl group and ** marks the attachment to the nitrogen atom of the adjacent piperidine ring and
R₄ represents hydrogen, methyl, ethyl, cyclopropyl, trifluoromethyl, bromine, chlorine, phenyl;
   where phenyl may be substituted by chlorine,
and the salts, solvates and solvates of the salts thereof.

In a possible subgroup of the compounds of formula I
X represents S,
Y represents N, and
Z represents C,
where in the resulting group of the formula (h),

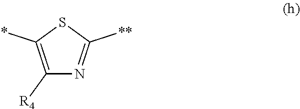

in which * denotes the bond to the carbonyl group and ** the bond to the N-atom of the adjacent piperidine-ring,
R₄ represents hydrogen or chloro.
and the salts, solvates and solvates of the salts thereof.

In another possible subgroup of the compounds of formula I
R₁ represents pyridinyl or phenyl,
   wherein pyridinyl may be substituted by 1 or 2 substituents independently selected from the group of methyl, ethyl, fluoro, chloro, trifluoromethyl and trifluoromethoxy;
   wherein phenyl may be substituted by 1 or 2 substituents independently selected from the group of methyl, cyclopropyl, methoxy, cyano, hydroxy, fluoro, chloro and trifluoromethyl,
and the salts, solvates and solvates of the salts thereof.

In another possible subgroup of the compounds of formula I
R₁ represents 3,5-difluoropyridin-2-yl.

In another possible subgroup of the compounds of formula I
R₂ represents hydrogen; or
   together with the carbon atom to which R₂ is attached, forms a cyclopropyl ring,
and the salts, solvates and solvates of the salts thereof.

In another possible subgroup of the compounds of formula I
R₆ represents a group of the formula a),

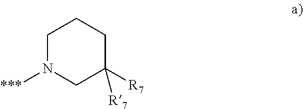

in which *** denotes the bond to the adjacent piperidine-ring, and
R₇ represents hydrogen,
R′₇ methyl, ethyl, n-propyl, iso-propyl, ethoxy, methoxymethyl, monofluoromethyl, difluoromethyl, trifluoromethyl, 3,3-difluorocyclobutylmethoxy, 2,2,2-trifluoroethoxymethyl, cyclopropylmethyl, 1-fluoromethylcyclopropylmethoxymethyl, 1-difluoromethylcyclopropylmethoxymethyl, 1-trifluoromethylcyclopropylmethoxymethyl, cyclobutylmethoxy, cyclopropylmethoxy, cyclobutyloxymethyl, cyclopropylmethoxymethyl, 3,3-difluorocyclobutylmethoxymethyl, 3-fluorobutyloxymethyl, 2.2-difluorocyclopropylmethoxy, cyclobutyloxy, 3.3-difluorocyclobutyloxy, 2-fluoorethyl, cyclopropyl, cyclobutyl, 2-methoxyethyl or tert-butyl, or
R₇ and R′₇ are attached to one another and, together with the carbon atom to which they are bonded, form a cyclopropyl ring,
and the salts, solvates and solvates of the salts thereof.

In another possible subgroup of the compounds of formula I
R₆ represents a group of the formula a),

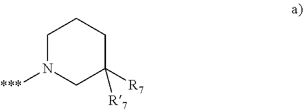

in which *** denotes the bond to the adjacent piperidine-ring, and
R₇ represents hydrogen,
R′₇ methyl, or
R₇ and R′₇ are attached to one another and, together with the carbon atom to which they are bonded, form a cyclopropyl ring,
and the salts, solvates and solvates of the salts thereof.

In another possible subgroup of the compounds of formula I n is 1.

In a further possible subgroup of the compounds of formula I m is 1.

In yet another possible subgroup of the compounds of formula I p is 1.

In yet another possible subgroup of the compounds of formula I q is 2.

In a further possible subgroup of the compounds of formula I the compound is N-[(3,5-difluoropyridin-2-yl)methyl]-2-[(3R)-3-methyl[1,4′-bipiperidin]-1′-yl]-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-[(3R*)-3-(methoxymethyl)[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide or N-[1-(3,5-difluoropyridin-2-yl)cyclopropyl]-2-[(3R)-3-methyl[1,4′-bipiperidin]-1′-yl]-1,3-thiazole-5-carboxamide,
and the salts, solvates and solvates of the salts thereof.

A preferred compound of formula (I) is N-[(3,5-difluoropyridin-2-yl)methyl]-2-[(3R)-3-methyl[1,4′-bipiperidin]-1′-yl]-1,3-thiazole-5-carboxamide,
and the salts, solvates and solvates of the salts thereof.

The compounds of formula (I), their production and their action as selective inhibitors of Adrenoreceptor ADRA2C or the treatment and/or prevention of breathing difficulties including sleep-induced breathing difficulties such as central and obstructive sleep apnoea, snoring (primary and obstructive snoring), dysphagia, peripheral and cardiac vascular disorders including diabetic microangiopathies and disorders of the peripheral and central nervous system including neurodegenerative and neuroinflammatory disorders are disclosed in WO 2021089683 A1 in general and especially the compounds specifically are an explicit part of the description of the present invention and are hereby incorporated by reference.

The term effective amount as used herein refers to an amount of a combination of compound of formula (I) and a muscarinic receptor antagonist that is effective for treatment and/or prophylaxis of sleep-related breathing disorders, preferably obstructive and central sleep apneas and snoring.

The present invention relates to combinations of compounds of formula (I) and a muscarinic receptor antagonist according to the invention for use in a method of treatment and/or prevention of respiratory disorders, sleep-related respiratory disorders, obstructive sleep apnoea, central sleep apnoea and snoring.

The present invention relates also to the use of combinations of compounds of formula (I) and a muscarinic receptor antagonist according to the invention for production of a medicament for treatment and/or prevention of respiratory disorders, sleep-related respiratory disorders, obstructive sleep apnoea, central sleep apnoea and snoring.

Moreover, the present invention relates to the use of one or more muscarinic receptor antagonists in combination with one or more α2-Adrenoceptor subtype C (alpha-2C) antagonists for preparing a pharmaceutical composition for the treatment sleep-related breathing disorders.

A further subject of the present invention is the use of a combination of compounds of formula (I) and a muscarinic receptor antagonist according to the invention with one or more other active compounds in a method for the treatment and/or prophylaxis sleep-related breathing disorders, preferably obstructive and central sleep apneas and snoring.

A further subject of the present invention is a medicament comprising at least one a combination of compounds of formula (I) and a muscarinic receptor antagonist according to the invention in combination with one or more inert non-toxic pharmaceutically suitable excipients for use in a method for the treatment and/or prophylaxis sleep-related breathing disorders, preferably obstructive and central sleep apneas and snoring.

The present invention further relates to a medicament comprising at least one a combination of compounds of formula (I) and a muscarinic receptor antagonist according to the invention with one or more other active compounds in combination with one or more inert non-toxic pharmaceutically suitable excipients for use in a method for the treatment and/or prophylaxis sleep-related breathing disorders, preferably obstructive and central sleep apneas and snoring.

The present invention is also directed to a method for the treatment and/or prophylaxis of sleep-related breathing disorders, by administering systemically and/or locally a therapeutically effective amount of at least one combination of compounds of formula (I) and a muscarinic receptor antagonist or a medicament comprising at least one combination of compounds of formula (I) and a muscarinic receptor antagonist according to the invention in combination with a inert, non-toxic, pharmaceutically acceptable additive.

Combination of compounds of formula (I) and a muscarinic receptor antagonist according to the invention can be used alone or, if required, in combination with one or more other pharmacologically active substances, provided that this combination does not lead to undesirable and unacceptable side effects. Preferred examples of combination suitable for the purpose to treat sleep-related breathing disorders, preferably obstructive and central sleep apneas and snoring, include:
respiratory stimulants such as, by way of example and with preference, theophylline, doxapram, nikethamide or caffeine;
noradrenaline reuptake inhibitors, by way of example and with preference, atomoxetine, reboxetine or desipramine;
5-HT2 receptor antagonists and serotonin reuptake inhibitor such as, by way of example and with preference, trazodone;
psychostimulants such as, by way of example and with preference, modafinil or armodafinil;
amphetamines and amphetamine derivatives such as, by way of example and with preference, amphetamine, metamphetamine or methylphenidate;
serotonin reuptake inhibitors such as, by way of example and with preference, fluoxetine, paroxetine, citalopram, escitalopram, sertraline or fluvoxamine;
serotonin precursors such as, by way of example and with preference, L-tryptophan;
noradrenergic and specific serotonergic antidepressants such as, by way of example and with preference, mirtazapine;
tricyclic antidepressants such as, by way of example and with preference, amitriptyline, protriptyline, doxepine, trimipramine, imipramine, clomipramine or desipramine;
GABA agonists such as, by way of example and with preference, baclofen;
glucocorticoids such as, by way of example and with preference, fluticasone, budesonide, beclometasone, mometasone, tixocortol or triamcinolone;
cannabinoid receptor agonists;
carboanhydrase inhibitors such as, by way of example and with preference, acetazolamide, methazolamide or diclofenamide;
opioid and benzodiazepine receptor antagonists such as, by way of example and with preference, flumazenil, naloxone or naltrexone;
cholinesterase inhibitors such as, by way of example and with preference, neostigmine, pyridostigmine, physostigmine donepezil, galantamine or rivastigmine;
appetite suppressants such as, by way of example and with preference, sibutramin, opiramate, phentermine, lipase inhibitors or cannabinoid receptor antagonists;
mineralocorticoid receptor antagonists.

A preferred subject of the present invention is a Medicament comprising combinations of compounds of formula (I) and a muscarinic receptor antagonist according to the invention in combination with one or more further active ingredients selected from the group consisting of noradrenaline reuptake inhibitors, 5-HT2 receptor antagonists, serotonin reuptake inhibitors, mineralocorticoid receptor antagonists, diuretics and corticosteroids.

A preferred subject of the present invention is a Medicament comprising combinations of compounds of formula (I) and a muscarinic receptor antagonist according to the invention for treatment and/or prevention of respiratory disorders, sleep-related respiratory disorders, obstructive sleep apnoea, central sleep apnoea and snoring

A preferred subject of the present invention is a combination comprising combinations of compounds of formula (I) and a muscarinic receptor antagonist according to the invention and one or more other active compounds selected from the groups consisting of noradrenaline reuptake inhibitors, 5-HT2 receptor antagonists, serotonin reuptake inhibitors, mineralocorticoid receptor antagonists, diuretics and corticosteroids for use in a method for the treatment and/or prophylaxis sleep-related breathing disorders, preferably obstructive and central sleep apneas and snoring.

Another preferred subject of the present invention is a medicament comprising combinations of compounds of formula (I) and a muscarinic receptor antagonist according to the invention in combination with one or more other active compounds selected from the groups consisting of noradrenaline reuptake inhibitors.

In a preferred embodiment of the invention, the combinations of the invention are administered in combination with a noradrenaline reuptake inhibitor, by way of example and with preference atomoxetine.

In a preferred embodiment of the invention, the combinations of the invention are administered in combination with a noradrenaline reuptake inhibitor, by way of example and with preference reboxetine.

In a preferred embodiment of the invention, the combinations of the invention are administered in combination with a noradrenaline reuptake inhibitor, by way of example and with preference desipramine.

Another preferred subject of the present invention is a medicament comprising combinations of compounds of formula (I) and a muscarinic receptor antagonist according to the invention in combination with one or more other active compounds selected from the groups consisting of 5-HT2 receptor antagonist and serotonin reuptake inhibitors.

In a preferred embodiment of the invention, the combinations of the invention are administered in combination with a 5-HT2 receptor antagonist and serotonin reuptake inhibitor, by way of example and with preference trazodone.

In a preferred embodiment of the invention, the combinations of the invention are administered in combination with a mineralocorticoid receptor antagonist, by way of example and with preference spironolactone, eplerenone or finerenone.

In a preferred embodiment of the invention, the combinations of the invention are administered in combination with a diuretic, by way of example and with preference furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

In a preferred embodiment of the invention, the compounds of the invention are administered in combination with a corticosteroid, by way of example and with preference prednisone, prednisolone, methylprednisolone, triamcinolone, dexamethasone, betamethasone, beclomethasone, flunisolide, budesonide or fluticasone.

If required, aryl piperazines of formula (I) according to the invention can also be employed in conjunction with the use of one or more medical technical devices or auxiliaries, provided this does not lead to unwanted and unacceptable side-effects. Medical devices and auxiliaries suitable for such a combined application are, by way of example and with preference:
devices for positive airway pressure ventilation such as, by way of example and with preference, CPAP (continuous positive airway pressure) devices, BiPAP (bilevel positive airway pressure) devices and IPPV (intermittent positive pressure ventilation) devices;
neurostimulators of the Nervus hypoglossus;
intraoral auxiliaries such as, by way of example and with preference, protrusion braces;
nasal disposable valves;
nasal stents.

Substituted heterocyclic carboxamides of formula (I) and muscarinic receptor antagonists according to the invention can act systemically and/or locally. For this purpose, they can be administered in a suitable manner, for example by the oral, parenteral, pulmonal, intrapulmonal (inhalative), nasal, intranasal, pharyngeal, lingual, sublingual, buccal, rectal, dermal, transdermal, conjunctival or otic route, or as an implant or stent.

A further subject of the present invention is a pharmaceutical composition comprising a combination of a compound of the formula (I) and a muscarinic receptor antagonist according to the invention for the systemic and/or local administration by the oral, parenteral, pulmonal, intrapulmonal (inhalative), nasal, intranasal, pharyngeal, lingual, sublingual, buccal, rectal, dermal, transdermal, conjunctival or otic route, or as an implant or stent. The preferred administration is the oral route.

For these administration routes, the compounds according to the invention can be administered in suitable administration forms.

For oral administration, administration forms which function according to the state of the art, releasing the compounds according to the invention rapidly and/or in a modified manner, which contain the compounds according to the invention in crystalline and/or amorphized and/or dissolved form, such as for example tablets (uncoated or coated tablets, for example with gastric juice-resistant or delayed dissolution or insoluble coatings, which control the release of the compound according to the invention), tablets rapidly disintegrating in the oral cavity or films/wafers, films/lyophilisates, capsules (for example hard or soft gelatine capsules), dragees, granules, pellets, powders, emulsions, suspensions, aerosols or solutions are suitable.

Parenteral administration can be effected omitting an absorption step (e.g. intravenous, intra-arterial, intracardial, intraspinal or intralumbar administration) or involving absorption (e.g. intra-muscular, subcutaneous, intracutaneous, percutaneous or intraperitoneal administration). Suitable administration forms for parenteral administration include injection and infusion preparations in the form of solutions, suspensions, emulsions, lyophilisates or sterile powders.

For the other administration routes, for example inhalation formulations (including powder inhalers and nebulisers), nasal drops, solutions or sprays, pharyngeal sprays, tablets for lingual, sublingual or buccal administration, tablets, films/wafers or capsules, suppositories, oral or ophthalmic preparations, vaginal capsules, aqueous suspensions (lotions, shakable mixtures), lipophilic suspensions, ointments, creams, transdermal therapeutic systems (e.g. plasters), milk, pastes, foams, dusting powders, implants or stents are suitable.

Oral administration is preferred.

The compounds according to the invention can be converted into the stated administration forms. This can be effected in a manner known per se by mixing with inert, non-toxic, pharmaceutically suitable additives. These additives include carriers (for example microcrystalline cellulose, lactose, mannitol), solvents (e.g. liquid polyethylene glycols), emulsifiers and dispersants or wetting agents (for example sodium dodecylsulphate, polyoxysorbitan oleate), binders (for example polyvinylpyrrolidone), synthetic and natural polymers (for example albumin), stabilizers (e.g. antioxidants such as for example ascorbic acid), colourants (e.g. inorganic pigments such as for example iron oxides) and flavour or odour correctors.

In general, to achieve effective results in oral administration it has been found advantageous to administer quantities of about 0.01 to 100 mg/kg, preferably about 0.01 to 10 mg/kg body weight. In nasal or pharyngeal administration, the dosage is about 0.01 μg/kg to 1000 μg/kg, preferably about 0.1 to 500 μg/kg body weight. Nonetheless it can sometimes be necessary to deviate from the said quantities, namely depending on body weight, administration route, individual response to the active substance, nature of the preparation and time or interval at which administration takes place. Thus in some cases it can be sufficient to manage with less than the aforesaid minimum quantity, while in other cases the stated upper limit must be exceeded. In the event of administration of larger quantities, it may be advisable to divide these into several individual administrations through the day.

The preferred administration is the oral route for a compound of of formula (I) and the oral route for the muscarinic receptor antagonist.

For oral administration, administration forms which function according to the state of the art, releasing the compounds according to the invention rapidly and/or in a modified manner, which contain the compounds according to the invention in crystalline and/or amorphized and/or dissolved form, such as for example tablets (uncoated or coated tablets, for example with gastric juice-resistant or delayed dissolution or insoluble coatings, which control the release of the compound according to the invention), tablets rapidly disintegrating in the oral cavity or films/wafers, films/lyophilisates, capsules (for example hard or soft gelatine capsules), dragees, granules, pellets, powders, emulsions, suspensions, aerosols or solutions are suitable.

C. Experimental Methods—Combination of an α2-Adrenoceptor Subtype C (Alpha-2C) Antagonists with a Muscarinic Receptor Antagonist

Advantageous pharmacological properties of the combination of an α2-Adrenoceptor subtype C (alpha-2C) antagonists with a muscarinic receptor antagonist can be determined by the following methods.

The therapeutic potential of the combination of an α2-Adrenoceptor subtype C (alpha-2C) antagonists with a muscarinic receptor antagonist according to the present invention in sleep apnea can be assessed preclinically in a pig model of obstructive sleep apnea (OSA).

Using negative pressure, it is possible to induce collapse and thus obstruction of the upper respiratory tract in anaesthetized, spontaneously breathing pigs (Wirth K. J. et al., Sleep 36(5) (2013) pp. 699-708).

German Landrace pigs are used for the model. The pigs are anaesthetized and tracheotomized. Two tracheal cannulas are inserted into the trachea, one into the rostral part and the other into the caudal part of the trachea. Using a connection piece, the rostral cannula is connected to a tube to the negative pressure device and to the distal tracheal cannula. The distal tracheal cannula is additionally connected to a tube with an open end to atmosphere via a connection piece that served for free tracheal breathing, circumventing the upper airway. By appropriate opening and clamping of those tubes breathing can be switched from nasal breathing to breathing through the caudal tracheal cannula, circumventing the upper airway, and the (isolated) upper airway can be connected to the negative pressure device, causing airflow in the inspiratory direction.

At certain points in time, the collapsibility of the upper respiratory tract is tested by having the pig breathe via the caudal cannula and applying negative pressures of −50, −100 and −150 cm water head (cm H2O) to the upper respiratory tract. This causes the upper respiratory tract to collapse, which manifests itself in an interruption of the airflow and a pressure drop in the tube system. This test is conducted prior to the administration of the test substance and at certain intervals after the administration of the test substance. An appropriately effective test substance can prevent this collapse of the respiratory tract in the inspiratory phase.

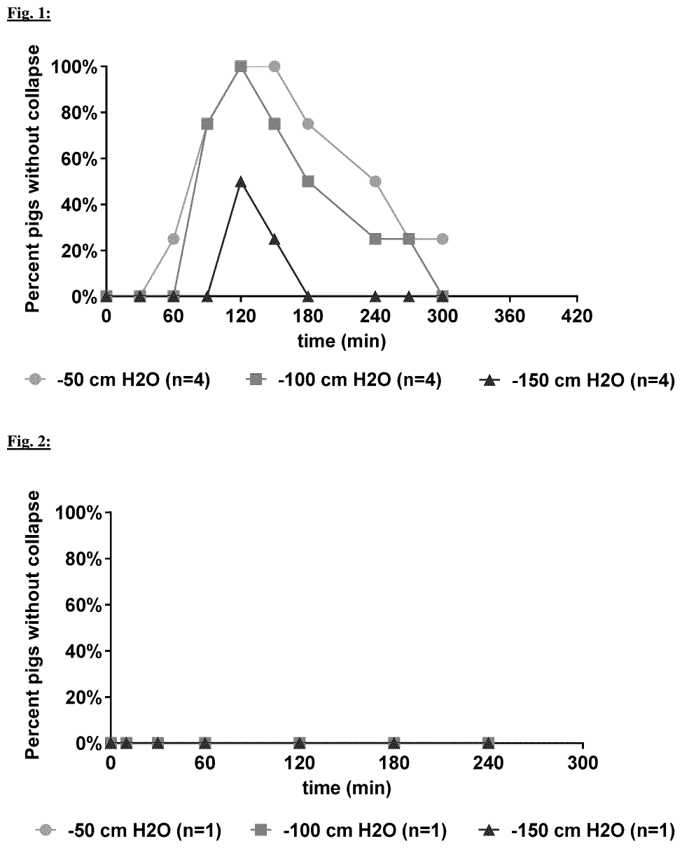

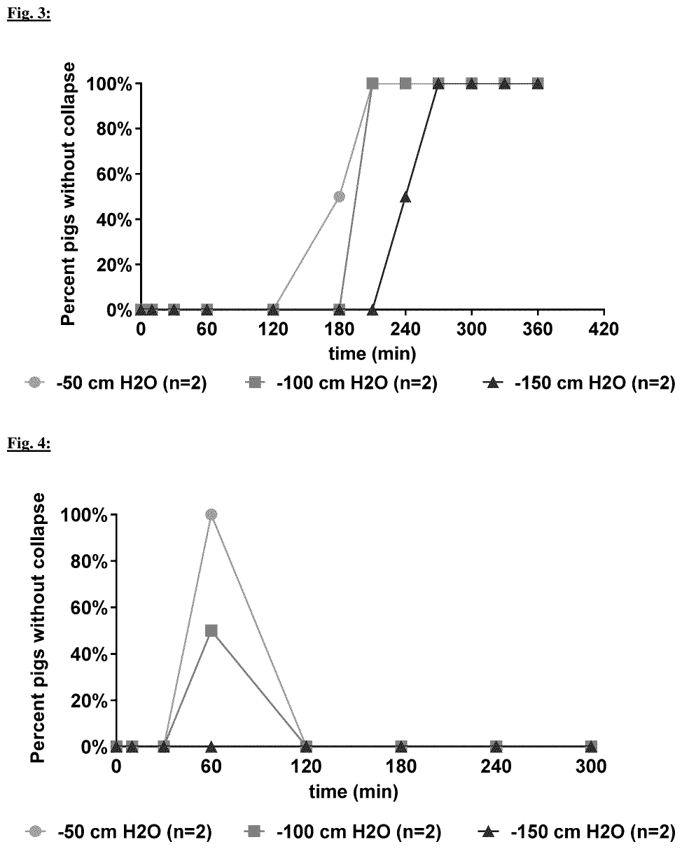

In this OSA pig model, systemic application of the α2-Adrenoceptor subtype C (alpha-2C) antagonists of formula (I), such as N-[(3,5-difluoropyridin-2-yl)methyl]-2-[(3R)-3-methyl[1,4′-bipiperidin]-1′-yl]-1,3-thiazole-5-carboxamide with intraduodenal administration of 0.01 mg/kg inhibited upper airway collapsibility at all negative pressures of −50, −100 and −150 cm head (cm H₂O) in all pigs at no time point after intraduodenal application. At time point 120 min after intraduodenal administration, upper airway collapsibility was inhibited at the negative pressures of −50 and −100 cm head (cm H2O) and at time point 150 min after intraduodenal administration, upper airway collapsibility was inhibited at the negative pressure of −50 cm head (cm H2O) (see Table 1, 2 and 3 and FIG. 1). Systemic application of the muscarinic receptor antagonist Oxybutynin via intravenous administration of a bolus injection of 1 mg/kg followed by an intravenous infusion of 0,275 mg/kg/h for four hours inhibited upper airway collapsibility at all negative pressures of −50, −100 and −150 cm head (cm H₂O) in all pigs at no time (see Table 4, 5 and 6 and FIG. 2). The combination of this non effective dose of the α2-Adrenoceptor subtype C (alpha-2C) antagonists of formula (I) N-[(3,5-difluoropyridin-2-yl)methyl]-2-[(3R)-3-methyl[1,4′-bipiperidin]-1′-yl]-1,3-thiazole-5-carboxamide with this non effective dose of the muscarinic receptor antagonist Oxybutynin inhibits upper airway collapsibility at all negative pressures of −50, −100 and −150 cm head (cm H₂O) for more than 90 min (see Table 7, 8 and 9 and FIG. 3).

FIG. 1: Effect of intraduodenal administration of 0.01 mg/kg of the α2-Adrenoceptor subtype C (alpha-2C) antagonists of formula (I) N-[(3,5-difluoropyridin-2-yl)methyl]-2-[(3R)-3-methyl[1,4′-bipiperidin]-1′-yl]-1,3-thiazole-5-carboxamide given at time point 0 min on upper airway collapsibility at different levels of negative pressure. Percentages of pigs with no collapse are given. Mean values.

TABLE 1
Intraduodenal administration of 0.01 mg/kg of the α2-Adrenoceptor
subtype C (alpha-2C) antagonists of formula (I) N-[(3,5-difluoropyridin-
2-yl)methyl]-2-[(3R)-3-methyl[1,4′-bipiperidin]-1′-yl]-1,3-
thiazole-5-carboxamide at negative pressures of −50 cm head (cm H₂O)

| | Percent pigs without |
|---|---|
| Time, min | collaps −50 cm H₂O, % |
| 0 | 0 |
| 30 | 0 |
| 60 | 25 |
| 90 | 75 |
| 120 | 100 |
| 150 | 100 |
| 180 | 75 |
| 240 | 50 |
| 270 | 25 |
| 300 | 25 |

TABLE 2
Intraduodenal administration of 0.01 mg/kg of the α2-Adrenoceptor
subtype C (alpha-2C) antagonists of formula (I) N-[(3,5-difluoropyridin-
2-yl)methyl]-2-[(3R)-3-methyl[1,4′-bipiperidin]-1′-yl]-1,3-
thiazole-5-carboxamide at negative pressures of −100 cm head (cm H₂O)

| | Percent pigs without |
|---|---|
| Time, min | collaps −100 cm H₂O, % |
| 0 | 0 |
| 30 | 0 |
| 60 | 0 |
| 90 | 75 |
| 120 | 100 |
| 150 | 75 |
| 180 | 50 |
| 240 | 25 |
| 270 | 25 |
| 300 | 0 |

TABLE 3
Intraduodenal administration of 0.01 mg/kg of the α2-Adrenoceptor
subtype C (alpha-2C) antagonists of formula (I) N-[(3,5-difluoropyridin-
2-yl)methyl]-2-[(3R)-3-methyl[1,4′-bipiperidin]-1′-yl]-1,3-
thiazole-5-carboxamide at negative pressures of −150 cm head (cm H₂O)

| | Percent pigs without |
|---|---|
| Time, min | collaps −150 cm H₂O, % |
| 0 | 0 |
| 30 | 0 |
| 60 | 0 |
| 90 | 0 |
| 120 | 50 |
| 150 | 25 |
| 180 | 0 |
| 240 | 0 |
| 270 | 0 |
| 300 | 0 |

Table 4, 5 and 6 and FIG. 2: Effect of intravenous administration of a bolus injection of 1 mg/kg followed by an intravenous infusion of 0.275 mg/kg/h for four hours of the muscarinic receptor antagonist oxybutynin given at time point 0 min on upper airway collapsibility at different levels of negative pressure. Percentages of pigs with no collapse are given. Mean values.

TABLE 4
Intravenous bolus injection of 1 mg/kg followed by
an intravenous infusion of 0.275 mg/kg/h for four
hours of the muscarinic receptor antagonist oxybutynin at
negative pressures of −50 cm head (cm H₂O)

| | Percent pigs without |
|---|---|
| Time, min | collaps −50 cm H₂O, % |
| 0 | 0 |
| 10 | 0 |
| 30 | 0 |
| 60 | 0 |
| 120 | 0 |
| 180 | 0 |
| 240 | 0 |

TABLE 5
Intravenous bolus injection of 1 mg/kg followed by
an intravenous infusion of 0.275 mg/kg/h for four
hours of the muscarinic receptor antagonist oxybutynin at
negative pressures of −100 cm head (cm H₂O)

| | Percent pigs without |
|---|---|
| Time, min | collaps −100 cm H₂O, % |
| 0 | 0 |
| 10 | 0 |
| 30 | 0 |
| 60 | 0 |
| 120 | 0 |
| 180 | 0 |
| 240 | 0 |

TABLE 6
Intravenous bolus injection of 1 mg/kg followed by
an intravenous infusion of 0.275 mg/kg/h for four
hours of the muscarinic receptor antagonist oxybutynin at
negative pressures of −150 cm head (cm H₂O)

| | Percent pigs without |
|---|---|
| Time, min | collaps −150 cm H₂O, % |
| 0 | 0 |
| 10 | 0 |
| 30 | 0 |
| 60 | 0 |
| 120 | 0 |
| 180 | 0 |
| 240 | 0 |

Table 7, 8 and 9 and FIG. 3: Effect of the non effective dose of N-[(3,5-difluoropyridin-2-yl)methyl]-2-[(3R)-3-methyl[11,4′-bipiperidin]-1′-yl]-1,3-thiazole-5-carboxamide with the non effective dose of the muscarinic receptor antagonist oxybutynin given at time point 0 min on upper airway collapsibility at different levels of negative pressure. Percentages of pigs with no collapse are given. Mean values.

TABLE 7
Combination of non effective dose of N-[(3,5-difluoropyridin-
2-yl)methyl]-2-[(3R)-3-methyl[1,4′-bipiperidin]-1′-yl]-1,3-
thiazole-5-carboxamide with the non effective dose of the muscarinic
receptor antagonist oxybutynin inhibits upper airway collapsibility
at negative pressures of −50 cm head (cm H₂O)

| | Percent pigs without |
|---|---|
| Time, min | collaps −50 cm H₂O, % |
| 0 | 0 |
| 30 | 0 |
| 60 | 0 |
| 120 | 0 |
| 180 | 50 |
| 210 | 100 |
| 240 | 100 |
| 270 | 100 |
| 300 | 100 |
| 360 | 100 |

TABLE 8
Combination of non effective dose of N-[(3,5-difluoropyridin-
2-yl)methyl]-2-[(3R)-3-methyl[1,4′-bipiperidin]-1′-yl]-1,3-
thiazole-5-carboxamide with the non effective dose of the muscarinic
receptor antagonist oxybutynin inhibits upper airway collapsibility
at negative pressures of −100 cm head (cm H₂O)

| | Percent pigs without |
|---|---|
| Time, min | collaps −100 cm H₂O, % |
| 0 | 0 |
| 30 | 0 |
| 60 | 0 |
| 120 | 0 |
| 180 | 0 |
| 210 | 100 |
| 240 | 100 |
| 270 | 100 |
| 300 | 100 |
| 360 | 100 |

TABLE 9
Combination of non effective dose of N-[(3,5-difluoropyridin-
2-yl)methyl]-2-[(3R)-3-methyl[1,4′-bipiperidin]-1′-yl]-1,3-
thiazole-5-carboxamide with the non effective dose of the muscarinic
receptor antagonist oxybutynin inhibits upper airway collapsibility
at negative pressures of −150 cm head (cm H₂O)

| | Percent pigs without |
|---|---|
| Time, min | collaps −150 cm H₂O, % |
| 0 | 0 |
| 30 | 0 |
| 60 | 0 |
| 120 | 0 |
| 180 | 0 |
| 210 | 0 |
| 240 | 50 |
| 270 | 100 |
| 300 | 100 |
| 360 | 100 |

In a second set of experiments in this OSA pig model, systemic application of the α2-Adrenoceptor subtype C (alpha-2C) antagonists of formula (I), such as N-[(3,5-difluoropyridin-2-yl)methyl]-2-[(3R)-3-methyl[1,4′-bipiperidin]-1′-yl]-1,3-thiazole-5-carboxamide with intraduodenal administration of 0.01 mg/kg inhibited upper airway collapsibility at all negative pressures of −50, −100 and −150 cm head (cm H₂O) in all pigs at no time point after intraduodenal application. At time point 120 min after intraduodenal administration, upper airway collapsibility was inhibited at the negative pressures of −50 and −100 cm head (cm H2O) and at time point 150 min after intraduodenal administration, upper airway collapsibility was inhibited at the negative pressure of −50 cm head (cm H2O) (see Table 1, 2 and 3 and FIG. 1). Systemic application of the muscarinic receptor antagonist R-Oxybutynin via intravenous administration of a bolus injection of 1 mg/kg followed by an intravenous infusion of 0,275 mg/kg/h for four hours inhibited upper airway collapsibility at all negative pressures of −50, −100 and −150 cm head (cm H₂O) in all pigs at no time. At time point 60 min after intravenous administration, upper airway collapsibility was inhibited at the negative pressure of −50 cm head (cm H2O) (see Table 10, 11 and 12 and FIG. 4). The combination of this non effective dose of the α2-Adrenoceptor subtype C (alpha-2C) antagonists of formula (I) N-[(3,5-difluoropyridin-2-yl)methyl]-2-[(3R)-3-methyl[1,4′-bipiperidin]-1′-yl]-1,3-thiazole-5-carboxamide with this non effective dose of the muscarinic receptor antagonist R-Oxybutynin inhibits upper airway collapsibility at all negative pressures of −50, −100 and −150 cm head (cm H₂O) for more than 300 min (see Table 13, 14 and 15 and FIG. 5).

Table 10, 11 and 12 and FIG. 4: Effect of intravenous administration of a bolus injection of 1 mg/kg followed by an intravenous infusion of 0.275 mg/kg/h for four hours of the muscarinic receptor antagonist R-oxybutynin given at time point 0 min on upper airway collapsibility at different levels of negative pressure. Percentages of pigs with no collapse are given. Mean values.

TABLE 10
Intravenous bolus injection of 1 mg/kg followed by
an intravenous infusion of 0.275 mg/kg/h for four
hours of the muscarinic receptor antagonist R-oxybutynin
at negative pressures of −50 cm head (cm H₂O)

| | Percent pigs without |
|---|---|
| Time, min | collaps −50 cm H₂O, % |
| 0 | 0 |
| 10 | 0 |
| 30 | 0 |
| 60 | 100 |
| 120 | 0 |
| 180 | 0 |
| 240 | 0 |

TABLE 11
Intravenous bolus injection of 1 mg/kg followed by
an intravenous infusion of 0.275 mg/kg/h for four
hours of the muscarinic receptor antagonist R-oxybutynin
at negative pressures of −100 cm head (cm H₂O)

| | Percent pigs without |
|---|---|
| Time, min | collaps −100 cm H₂O, % |
| 0 | 0 |
| 10 | 0 |
| 30 | 0 |
| 60 | 50 |
| 120 | 0 |
| 180 | 0 |
| 240 | 0 |

TABLE 12
Intravenous bolus injection of 1 mg/kg followed by
an intravenous infusion of 0.275 mg/kg/h for four
hours of the muscarinic receptor antagonist R-oxybutynin
at negative pressures of −150 cm head (cm H₂O)

| | Percent pigs without |
|---|---|
| Time, min | collaps −150 cm H₂O, % |
| 0 | 0 |
| 10 | 0 |
| 30 | 0 |
| 60 | 0 |
| 120 | 0 |
| 180 | 0 |
| 240 | 0 |

Table 13, 14 and 15 and FIG. 5: Effect of the non effective dose of N-[(3,5-difluoropyridin-2-yl)methyl]-2-[(3R)-3-methyl[1,4′-bipiperidin]-1′-yl]-1,3-thiazole-5-carboxamide with the non effective dose of the muscarinic receptor antagonist R-oxybutynin given at time point 0 min on upper airway collapsibility at different levels of negative pressure. Percentages of pigs with no collapse are given. Mean values.

TABLE 13
Combination of non effective dose of N-[(3,5-difluoropyridin-
2-yl)methyl]-2-[(3R)-3-methyl[1,4′-bipiperidin]-1′-yl]-1,3-
thiazole-5-carboxamide with the non effective dose of the muscarinic
receptor antagonist R-oxybutynin inhibits upper airway collapsibility
at negative pressures of −50 cm head (cm H₂O)

| | Percent pigs without |
|---|---|
| Time, min | collaps −50 cm H₂O, % |
| 0 | 0 |
| 30 | 100 |
| 60 | 100 |
| 90 | 100 |
| 180 | 100 |
| 240 | 100 |
| 300 | 100 |
| 360 | 100 |

TABLE 14
Combination of non effective dose of N-[(3,5-difluoropyridin-
2-yl)methyl]-2-[(3R)-3-methyl[1,4′-bipiperidin]-1′-yl]-1,3-
thiazole-5-carboxamide with the non effective dose of the muscarinic
receptor antagonist R-oxybutynin inhibits upper airway collapsibility
at negative pressures of −100 cm head (cm H₂O)

| | Percent pigs without |
|---|---|
| Time, min | collaps −100 cm H₂O, % |
| 0 | 0 |
| 30 | 33 |
| 60 | 100 |
| 90 | 100 |
| 180 | 100 |
| 240 | 100 |
| 300 | 100 |
| 360 | 100 |

TABLE 15
Combination of non effective dose of N-[(3,5-difluoropyridin-
2-yl)methyl]-2-[(3R)-3-methyl[1,4′-bipiperidin]-1′-yl]-1,3-
thiazole-5-carboxamide with the non effective dose of the muscarinic
receptor antagonist R-oxybutynin inhibits upper airway collapsibility
at negative pressures of −150 cm head (cm H₂O)

| | Percent pigs without |
|---|---|
| Time, min | collaps −150 cm H₂O, % |
| 0 | 0 |
| 30 | 0 |
| 60 | 100 |
| 90 | 100 |
| 180 | 100 |
| 240 | 100 |
| 300 | 100 |
| 360 | 100 |

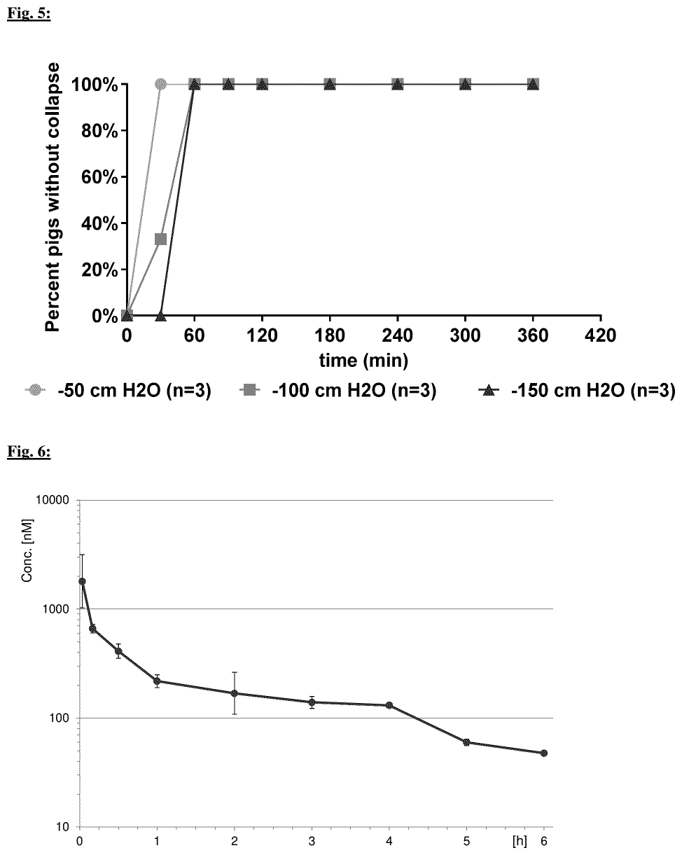

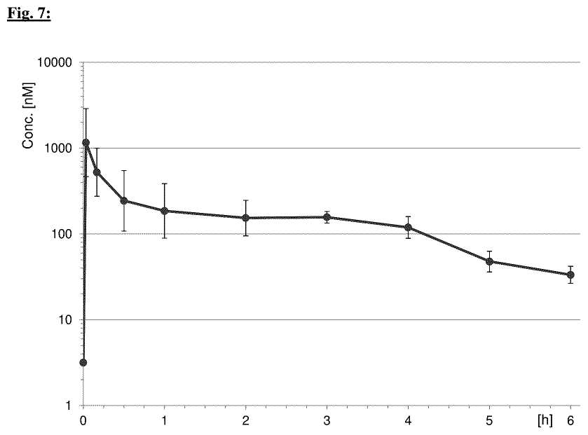

It has been found that the combination of the non effective dose of 0.01 mg/kg of the adrenoreceptor ADRA2C inhibitor with a non effective dose of the muscarinic receptor antagonists oxybutynin, reaching plasma concentrations of about 100-500 nM (FIG. 6), or R-Oxybutynin, reaching plasma concentrations of about 100-300 nM (FIG. 7), inhibits upper airway collapsibility with synergistic efficacy compared to each treatment alone. From the above mentioned data it can be deducted that the combination of an adrenoreceptor ADRA2C inhibitor of formula (I) with a muscarinic receptor antagonist inhibits upper airway collapsibility with synergistic efficacy compared to each treatment alone and is thus suitable to treat sleep-related breathing disorders, preferably obstructive and central sleep apneas and snoring.

1. Combinations of compounds of formula (I) 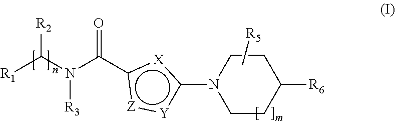
in which
X represents S, N or O;
Y represents N, S or O,
where, if X represents S, then Y represents N;
where, if X represents 0, then Y represents N;
Z represents CR₄, O or NR₄,
where, if X represents N and Y represents N, then Z represents 0;
where, if X represents S, then Z represents CR₄ or NR₄
R₁ represents 5- or 6-membered heteroaryl, phenyl,
where 5- to 6-membered heteroaryl may be substituted by 1 to 2 substituents independently of one another selected from the group of (C₁-C₄)-alkyl, (C₁-C₄)-alkoxy, halogen;
where (C₁-C₄)-alkyl may be up to trisubstituted by halogen,
where (C₁-C₄)-alkoxy may be up to trisubstituted by halogen,
where phenyl may be substituted by 1 to 2 substituents independently of one another selected from the group of (C₁-C₄)-alkyl, (C₃-C₅)-cycloalkyl, (C₁-C₄)-alkoxy, cyano, hydroxy, halogen;
where (C₁-C₄)-alkyl may be up to trisubstituted by halogen,
R₂ represents hydrogen, (C₁-C₄)-alkyl;
where (C₁-C₄)-alkyl may be up to trisubstituted by halogen, or
together with the carbon atom to which R₂ is attached forms a (C₃-C₄)-cycloalkyl ring,
R₃ represents hydrogen, (C₁-C₄)-alkyl,
where (C₁-C₄)-alkyl may be up to trisubstituted by halogen,
R₄ in CR₄ represents hydrogen, (C₁-C₄)-alkyl, (C₃-C₄)-cycloalkyl, phenyl, halogen;
where (C₁-C₄)-alkyl may be up to trisubstituted by halogen and phenyl may be substituted by halogen,
in NR₄ represents hydrogen, (C₁-C₄)-alkyl, (C₃-C₄)-cycloalkyl, phenyl;
where (C₁-C₄)-alkyl may be up to trisubstituted by halogen and phenyl may be substituted by halogen,
R₅ represents hydrogen, (C₁-C₄)-alkyl, (C₁-C₄)-alkoxy, halogen,
R₆ represents a group of formula a), b), c), d), e), f) or g) 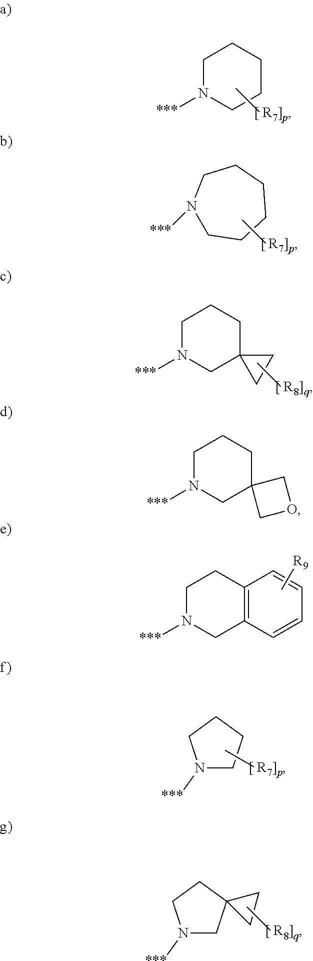
where *** marks the attachment to the adjacent piperidine ring,
where R₇ represents hydrogen, (C₁-C₄)-alkyl, (C₃-C₄)-cycloalkyl, (C₁-C₄)-alkoxy, (C₃-C₄)-cycloalkoxy, phenyl,
where (C₁-C₄)-alkyl may be substituted by (C₃-C₄)-cycloalkyl, (C₁-C₄)-alkoxy, (C₃-C₄)-cycloalkoxy and up to trisubstituted by halogen,
where (C₁-C₄)-alkoxy may be substituted by (C₃-C₄)-cycloalkyl and up to trisubstituted by halogen,
where (C₃-C₄)-cycloalkyl may be substituted by monofluoromethyl, difluoromethyl or trifluoromethyl and up to disubstituted by halogen,
where (C₁-C₄)-alkoxy may be substituted by (C₃-C₄)-cycloalkyl and up to trisubstituted by halogen,
where (C₃-C₄)-cycloalkyl may be mono- or disubstituted by halogen,
where (C₃-C₄)-cycloalkoxy may be up to disubstituted by halogen,
where R₈ represents hydrogen or fluorine,
where R₉ represents hydrogen, (C₁-C₄)-alkyl, (C₁-C₄)-alkoxy, halogen;
where (C₁-C₄)-alkyl may be substituted by (C₁-C₄)-alkoxy,
n represents 0 or 1,
m represents 0, 1 or 2,
p represents 0, 1 or 2 and
q represents 0, 1 or 2, and
a muscarinic receptor antagonist,
and the salts, solvates and solvates of the salts thereof.

2. The combinations according to claim 1 of compounds of formula (I),
in which
X, Y and Z are selected such that the aromatic 5-membered ring has the structural formula h), i), j), k) or (r), 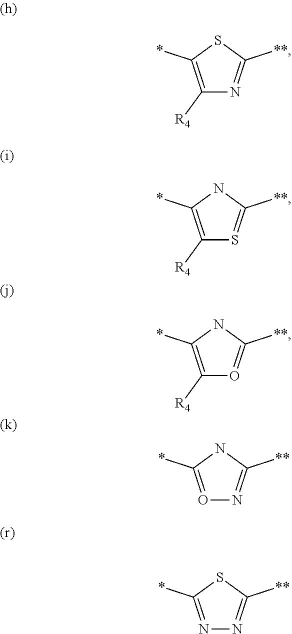
where * marks the attachment to the carbonyl group and ** marks the attachment to the nitrogen atom of the adjacent piperidine ring and
R₁ represents pyridinyl, pyrazolyl, thiazolyl, thienyl, phenyl,
where pyridinyl may be substituted by 1 to 2 substituents independently of one another selected from the group of (C₁-C₂)-alkyl, fluorine, chlorine, trifluoromethyl, trifluoromethoxy,
where pyrazolyl may be substituted by 1 to 2 substituents independently of one another selected from the group of (C₁-C₂)-alkyl, fluorine, chlorine, trifluoromethyl,
where thiazolyl may be substituted by chlorine,
where thienyl may be substituted by fluorine,
where phenyl may be substituted by 1 to 2 substituents independently of one another selected from the group of (C₁-C₂)-alkyl, (C₃-C₄)-cycloalkyl, methoxy, cyano, hydroxy, fluorine, chlorine, trifluoromethyl;
R₂ represents hydrogen, methyl, or
together with the carbon atom to which R₂ is attached forms a cyclopropyl ring,
R₃ represents hydrogen, (C₁-C₂)-alkyl;
R₄ represents hydrogen, methyl, ethyl, cyclopropyl, trifluoromethyl, bromine, chlorine, phenyl;
where phenyl may be substituted by chlorine,
R₅ represents hydrogen, fluorine;
R₆ represents a group of the formula a), b′), b″), c′), c″) or e), 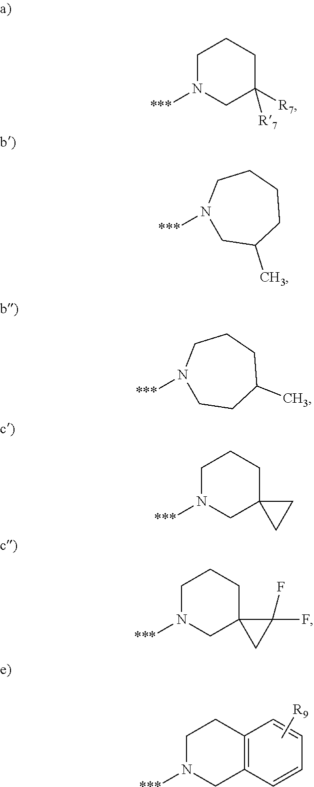
where *** marks the attachment to the adjacent piperidine ring,
where R₇ or R′₇ independently of one another represent hydrogen, (C₁-C₄)-alkyl, (C₃-C₄)-cycloalkyl, (C₁-C₂)-alkoxy, (C₃-C₄)-cycloalkoxy, monofluoromethyl, difluoromethyl, trifluoromethyl, difluoromethoxy, phenyl,
where (C₁-C₄)-alkyl may be substituted by methoxy, n-butoxy, cyclopropyl, cyclobutoxy and up to disubstituted by fluorine,
where methoxy may be substituted by cyclopropyl, cyclobutyl, trifluoromethyl,
where cyclopropyl may be substituted by monofluoromethyl, difluoromethyl, trifluoromethyl,
where cyclobutyl may be up to disubstituted by fluorine,
where n-butoxy may be up to disubstituted by fluorine,
where (C₁-C₂)-alkoxy may be substituted by cyclopropyl, cyclobutyl, cyclobutoxy, trifluoromethyl and
where cyclopropyl and cyclobutyl may be up to disubstituted by fluorine,
where (C₃-C₄)-cycloalkoxy may be up to disubstituted by fluorine,
where R₉ represents hydrogen, methyl, tert-butyl, methoxy, methoxymethyl, fluorine, chlorine;
n represents 0 or 1 and
m represents 1 or 2, and
a muscarinic receptor antagonist,
and the salts, solvates and solvates of the salts thereof.

3. The combinations according to claim 1, in which
X, Y and Z are selected from the group of S, N, O and C to form 1,3-thiazolyl, 1,3-oxazolyl, or 1,2,4-oxadiazolyl;
R₁ represent pyridinyl, 2-ethylpyridinyl, 4,6-dimethylpyridinyl, 3,5-difluoropyridinyl, 3-fluoropyridinyl, 4-trifluoromethylpyridinyl, 6-trifluoromethylpyridinyl, 5-chloro-3-fluoropyridinyl, 3-chloro-5-fluoropyridinyl, 3-methylpyridinyl, 4-methylpyridinyl, 6-methylpyridinyl 3-chloropyridinyl, 5-chloropyridinyl, 6-trifluoromethoxypyridinyl, phenyl, 2-methylphenyl, 3-methylphenyl, 4-methylphenyl, 3-methoxyphenyl, 4-trifluoromethylphenyl, 2-chlorophenyl, 3-chlorophenyl, 4-chlorophenyl, 2-fluorophenyl, 3-fluorophenyl, 4-fluorophenyl, 3-hydroxyphenyl, 2,5-difluorophenyl, 5-chloro-2-hydroxyphenyl, 5-fluoro-2-methoxyphenyl, 5-chloro-2-fluorophenyl, 2-chloro-5-fluorophenyl, 2-chloro-4-fluorophenyl, 3-cyano-4-fluorophenyl, 2-cyclopropylphenyl, 4-chloro-1-methyl-1H-pyrazolyl, 5-chloro-1,3-thiazolyl, 5-fluoro-2-thienyl;
R₂ represents hydrogen or methyl;
R₃ represents hydrogen or methyl;
R₄ represents hydrogen, methyl, ethyl or trifluormethyl;
R₅ represents hydrogen or fluoro;
R₆ represents a group of the formula a), c′) or c″), 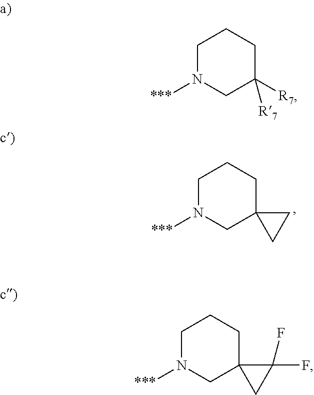
in which *** marks the bond to the adjacent piperidine ring,
wherein R₇ or R′₇ independently from each other represent hydrogen, methyl, ethyl, n-propyl, iso-propyl, tert-butyl, 2-fluoroethyl, cyclopropyl, cyclobutyl, cyclopropylmethyl, methoxy, ethoxy, methoxymethyl, monofluoromethyl, difluoromethyl, trifluormethyl, difluormethoxy, 3,3-difluorocyclobutylmethoxy, cyclobutylmethoxy, cyclopropylmethoxy, cyclopropyl-methoxymethyl, cyclobutyloxymethyl, 3-fluorobutyloxymethyl, 3,3-difluorocyclobutyl-methoxymethyl, 2,2,2-trifluoroethoxy, 2,2,2-trifluoroethoxymethyl, 2,2-difluorocyclopropyl-methoxy, cyclobutyloxy, 3,3-difluorocyclobutyloxy, fluoromethylcyclopropylmethoxy, difluoromethylcyclopropylmethoxy, trifluoromethylcyclopropylmethoxy or fluoro;
n represents 0 or 1,
m represents 1, and
a muscarinic receptor antagonist selected from the group selected from the group comprising Oxybutynin, R-Oxybutynin and Tolterodine,
and the salts, solvates and solvates of the salts thereof.

4. The combinations according to claim 1, wherein the compound of formula (I)
is selected from the group consisting of
N-[(3,5-difluoropyridin-2-yl)methyl]-2-[(3R)-3-methyl[1,4′-bipiperidin]-1′-yl]-1,3-thiazole-5-carboxamide, 2-[4-(5-azaspiro[2.5]octan-5-yl)piperidin-1-yl]-N-[(3,5-difluoropyridin-2-yl)methyl]-1,3-thiazole-5-carboxamide, N-[(3,5-difluoropyridin-2-yl)methyl]-2-[(3R*)-3-(methoxymethyl)[1,4′-bipiperidin]-1′-yl]-1,3-thiazole-5-carboxamide, 4-chloro-N-[(3,5-difluoropyridin-2-yl)methyl]-2-[(3R)-3-methyl[1,4′-bipiperidin]-1′-yl]-1,3-thiazole-5-carboxamide and N-[1-(3,5-difluoropyridin-2-yl)cyclopropyl]-2-[(3R)-3-methyl[1,4′-bipiperidin]-1′-yl]-1,3-thiazole-5-carboxamide, and
a muscarinic receptor antagonist selected from the group selected from the group comprising Oxybutynin, R-Oxybutynin and Tolterodine,
and the salts, solvates and solvates of the salts thereof.

5. The combinations according to claim 1, wherein the compound of formula (I) is N-[(3,5-difluoropyridin-2-yl)methyl]-2-[(3R)-3-methyl[1,4′-bipiperidin]-1′-yl]-1,3-thiazole-5-carboxamide,
and a muscarinic receptor antagonist selected from the group selected from the group comprising Oxybutynin, R-Oxybutynin and Tolterodine,
and the salts, solvates and solvates of the salts thereof.

6. The combinations according to claim 1, wherein the compound of formula (I) is N-[(3,5-difluoropyridin-2-yl)methyl]-2-[(3R)-3-methyl[1,4′-bipiperidin]-1′-yl]-1,3-thiazole-5-carboxamide- and Oxybutynin,
and the salts, solvates and solvates of the salts thereof.

7. The combinations according to claim 1, wherein the compound of formula (I) is N-[(3,5-difluoropyridin-2-yl)methyl]-2-[(3R)-3-methyl[1,4′-bipiperidin]-1′-yl]-1,3-thiazole-5-carboxamide and R-Oxybutynin, and the salts, solvates and solvates of the salts thereof.

8. The combinations as defined in claim 1 for use in a method of treatment and/or prevention of respiratory disorders, sleep-related respiratory disorders, obstructive sleep apnea, central sleep apnea and snoring.

9. A use of combination as defined in claim 1 for production of a medicament for treatment and/or prevention of respiratory disorders, sleep-related respiratory disorders, obstructive sleep apnea, central sleep apnea and snoring.

10. A medicament comprising combinations as defined in claim 1 in combination with one or more inert, nontoxic, pharmaceutically suitable excipients.

11. The medicament comprising combinations as defined in claim 1 in combination with one or more further active ingredients selected from the group consisting of noradrenaline reuptake inhibitors, 5-HT2 receptor antagonists, serotonin reuptake inhibitors, mineralocorticoid receptor antagonists, diuretics and corticosteroids.

12. The medicament according to claim 10 for treatment and/or prevention of respiratory disorders, sleep-related respiratory disorders, obstructive sleep apnea, central sleep apnea and snoring.

13. A method of treatment and/or prevention of respiratory disorders, sleep-related respiratory disorders, obstructive sleep apnea, central sleep apnea and snoring in humans and animals by administration of an effective amount of at least one combination or of a medicament as defined in claim 10.

14. A use according to claim 8, wherein the sleep-related breathing disorders are obstructive sleep apnea and central sleep apnea and snoring.
